(19) United States
(12) Patent Application Publication
(10) Pub. No.: US 2024/0390505 A1
(43) Pub. Date: Nov. 28, 2024

(54) CONJUGATES, COMPOSITIONS, AND METHODS FOR REJUVENATION OF CAR T-CELLS

(71) Applicant: Purdue Research Foundation, West Lafayette, IN (US)

(72) Inventors: Philip Stewart LOW, West Lafayette, IN (US); John Victor NAPOLEON, West Lafayette, IN (US)

(21) Appl. No.: 18/687,785

(22) PCT Filed: Aug. 30, 2022
(86) PCT No.: PCT/US2022/042094

§ 371 Date: Feb. 28, 2024

Related U.S. Application Data
(60) Provisional application No. 63/238,300, filed on Aug. 30, 2021.

(51) Int. Cl. A61K 47/55; A61K 39/00; A61K 47/60

(52) U.S. Cl. CPC A61K 47/551; A61K 39/4611; A61K 39/4631; A61K 39/4646; A61K 47/55; A61K 47/60; A61K 2239/38

(57) ABSTRACT

Chimeric antigen receptor (CAR) T-cell-rejuvenating conjugates and compositions and methods of use to rejuvenate exhausted CAR T-cells.

### PRIORITY

This patent application is related to and claims the priority benefit of U.S. Provisional Patent Application No. 63/238,300 filed Aug. 30, 2021. The content of the foregoing application is hereby incorporated by reference in its entirety into this disclosure.

### TECHNICAL FIELD

This disclosure relates to chimeric antigen receptor (CAR) T-cells, conjugates comprising Toll-like receptor (TLR) 7/8 agonists (i.e., TLR7, TLR8, or TLR7 and TLR8), compositions comprising the same, and methods of use thereof to rejuvenate exhausted CAR T-cells.

### BACKGROUND

Chimeric antigen receptor (CAR) T-cells have demonstrated significant promise in the treatment of hematopoietic cancer. Their usefulness to eradicate solid tumors, however, is compromised by their development of a dysfunctional or exhausted phenotype, leading to decreased proliferation and compromised effector function.

In view of the above, there is an unmet need to rejuvenate exhausted CAR T-cells. Desirably, the rejuvenation of CAR T-cells is targeted, thereby minimizing, if not eliminating, toxicity to healthy cells.

It is an object of the present disclosure to provide conjugates, compositions, and methods for rejuvenating exhausted CAR T-cells. This and other objects, as well as inventive features and advantages, will be apparent from the description provided herein.

### SUMMARY

Provided is a method of rejuvenating exhausted chimeric antigen receptor (CAR) T-cells in a subject with a cancer being treated with: (a) (i) a vector comprising a promoter operably linked to a nucleic acid sequence encoding a CAR or (ii) T-cells expressing the CAR, and (b) a cancer-binding conjugate comprising a ligand that binds a cancer cell with specificity and conjugated with the first targeting moiety. The CAR binds a first targeting moiety, a second targeting moiety, or both of the first targeting moiety and the second targeting moiety, and the ligand that binds the cancer cell with specificity and the first targeting moiety can be optionally conjugated via a first linker. The method can comprise:
administering to the subject (e.g., with exhausted CAR T-cells) a CAR T-cell-rejuvenating conjugate comprising an agonist of toll-like receptor 7 (TLR7), toll-like receptor 8 (TLR8), or toll-like receptor 7 and toll-like receptor 8 (TLR7/8) conjugated with the first targeting moiety or the second targeting moiety, wherein the agonist and either of the first targeting moiety or the second targeting moiety are conjugated via a second linker.

The CAR T-cell-rejuvenating conjugate can have the structure:

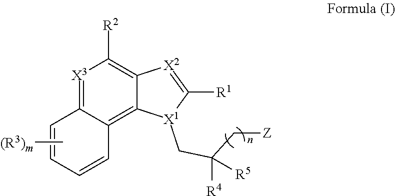

or be a pharmaceutically acceptable salt thereof, wherein:
   R¹ is an acyclic or cyclic alkyl, which is optionally substituted with one or more substituents independently selected from an alkyl, halo, heteroalkyl, alkoxy, and cycloalkyl;
   R² is —NR^{2x}R^{2y}, hydrogen (H), —OR^{z}, —SO₂N(R^{z})₂, or N₃, wherein:
      R^{2x} and R^{2y} are each independently H, —N(R^{z})₂, —CON(R^{z})₂, —C(R^{z})₂—N(R^{z})₂, —CS—N(R^{z})₂, or an alkyl that is optionally substituted with one or more substituents independently selected from oxo, halo, alkyl, heteroalkyl, alkoxy, and cycloalkyl;
      each R^{Z} is independently H or an alkyl, which is optionally substituted; or
      R^{2x} and R^{2y} are taken together to form a 3-10 membered mono- or bicyclic heterocycloalkyl, which is optionally substituted;
   each R³ is independently H, halo, —N₃, —CN, —NO₂, alkyl, heteroalkyl, cycloalkyl, heterocycloalkyl, alkoxy, aryl, heteroaryl, heterocycloalkyl, amino, hydroxy, carbonyl, or thiol, wherein the alkyl, heteroalkyl, cycloalkyl, heterocycloalkyl, or alkoxy that is optionally substituted;
   R⁴ and R⁵ are each independently alkyl, alkoxy, halo, or cycloalkyl, wherein the alkyl, alkoxy, or cycloalkyl is optionally substituted;
   X¹, X², and X³ are independently N or CR^{q}, wherein each R^{q} is independently hydrogen, halo, or optionally substituted alkyl;
   Z is G-L-, wherein L is a linker (e.g., the second linker) and G is a targeting moiety (e.g., the first targeting moiety or the second targeting moiety);
   n in Formula (I) is 1-6; and
   m in Formula (I) is 0-4.

The CAR T-cell-rejuvenating conjugate can have the structure:

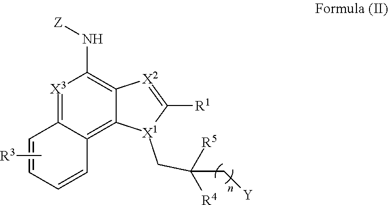

or be a pharmaceutically acceptable salt thereof, wherein:
   R¹, R³, R⁴, and R⁵ are each independently H, alkyl, alkoxyl, alkenyl, alkynyl, alicyclic, aryl, biaryl, halo, heteroaryl, —COR^{2x}

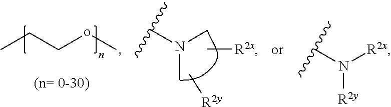

wherein each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl;
   Z is G-L-, wherein L is a linker (e.g., L is the second linker) and G is a targeting moiety (e.g., G is the first targeting moiety or the second targeting moiety);
   X¹, X², and X³ are independently CR^{q} or N, wherein each R^{q} is independently H, halo, or optionally substituted alkyl;
   Y is H, —OR^{z}, —NR^{2x}R^{2y}, —SR^{z}, —SOR^{z}, —SO₃R^{z}, —N₃, —COR^{z}, —COOR^{z}, —CON(R^{z})₂, —COSR^{z}, —SO₂N(R^{z})₂, or —CON(R^{z})₂, wherein:
      R^{2x} and R^{2y} are each independently H, —N(R^{z})₂, —CON(R^{z})₂, —C(R^{z})₂—N(R^{z})₂, —CS—N(R^{z})₂, or an optionally substituted alkyl (e.g., optionally substituted with one or more substituent, each substituent independently being oxo, halo, alkyl, heteroalkyl, alkoxy, or cycloalkyl);
      each R^{Z} is independently H or an optionally substituted alkyl; or
      R^{2x} and R^{2y} are taken together to form an optionally substituted heterocycloalkyl (e.g., wherein the optionally substituted heterocycloalkyl is a mono- or bicyclic heterocycloalkyl and/or wherein the optionally substituted heterocycloalkyl is a 3-10 membered heterocycloalkyl); and
   n in Formula (II) is 0-30.

In certain embodiments, the CAR T-cell-rejuvenating conjugate can have the structure:

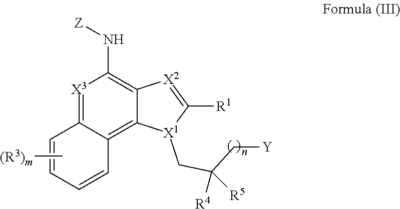

or is a pharmaceutically acceptable salt thereof, wherein:
   R¹ is an acyclic or cyclic alkyl, which is optionally substituted with one or more substituents independently selected from halo, alkyl, heteroalkyl, alkoxy, and cycloalkyl;
   Y is H, —OR^{z}, —NR^{2x}R^{2y}, —SR^{z}, —SOR^{Z}, —SO₃R^{z}, —N₃, —COR^{z}, —COOR^{z}, —CON(R^{z})₂, —COSR^{z}, —SO₂N(R^{z})₂, or —CON(R^{z})₂, wherein R^{2x} and R^{2y} are each independently H, —N(R^{z})₂, —CON(R^{z})₂, —C(R^{z})₂—N(R^{z})₂, —CS—N(R^{z})₂, or an optionally substituted alkyl (e.g., optionally substituted with one or more substituent, each substituent independently being oxo, halogen, alkyl, heteroalkyl, alkoxy, or cycloalkyl);
   each R^{Z} is independently H or an optionally substituted alkyl; or
   R^{2x} and R^{2y} are taken together to form an optionally substituted heterocycloalkyl (e.g., wherein the optionally substituted heterocycloalkyl is a mono- or bicyclic heterocycloalkyl and/or wherein the optionally substituted heterocycloalkyl is a 3-10 membered heterocycloalkyl);
   each R³ is independently halo, —N₃, —CN, —NO₂, alkyl, heteroalkyl, cycloalkyl, heterocycloalkyl, alkoxy, aryl, heteroaryl, heterocycloalkyl, amino, hydroxy, carbonyl, or thiol, wherein the alkyl, alkoxy, heteroalkyl, cycloalkyl, or heterocycloalkyl is optionally substituted;
   R⁴ and R⁵ are each independently alkyl, alkoxy, halogen, or cycloalkyl, wherein the alkyl, alkoxy, or cycloalkyl is optionally substituted;
   X¹, X², and X³ are independently CR^{q} or N, wherein each R^{q} is independently H, halogen, or an optionally substituted alkyl;
   Z is L-G, wherein L is a linker (e.g., the second linker) and G is a targeting moiety (e.g., the first targeting moiety or the second targeting moiety);
   n in Formula (III) is 0-30; and
   m in Formula (III) is 0-4.

In use, CAR T-cells in the subject (e.g., exhausted CAR T-cells) can bind and endocytose the CAR T-cell-rejuvenating conjugate such that the T-cells are rejuvenated by the agonist of the CAR T-cell-rejuvenating conjugate.

In various embodiments, each of X¹, X², and X³ of Formula (I), Formula (II), or Formula (III) can be N.

The CAR T-cell-rejuvenating conjugate can have the structure:

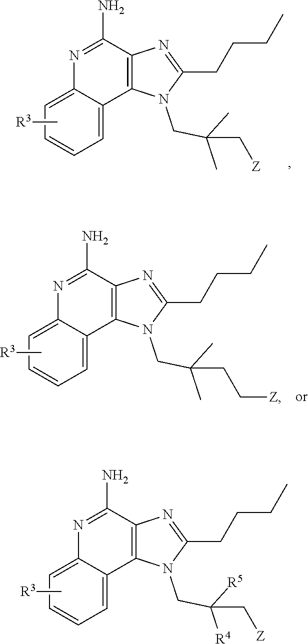

or be a pharmaceutically acceptable salt of any of the foregoing structures. Alternatively, the CAR T-cell-rejuvenating conjugate can have the structure:

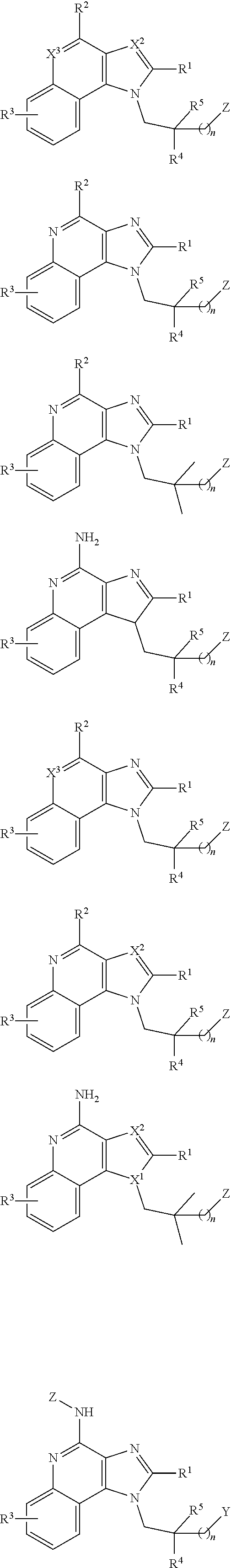

or be a pharmaceutically acceptable salt of any of the foregoing structures.

In certain embodiments, the agonist of the CAR T-cell-rejuvenating conjugate is an agonist of TLR7/8 and has the structure:

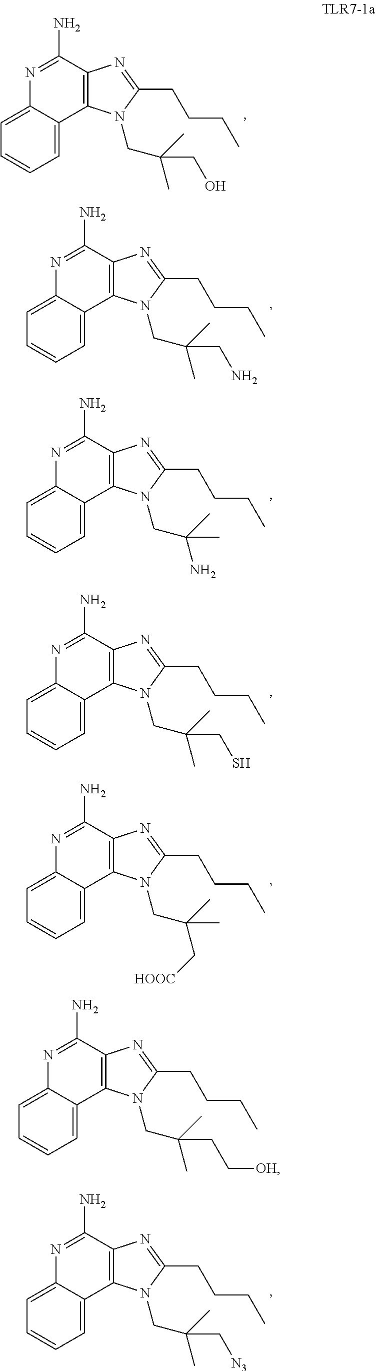

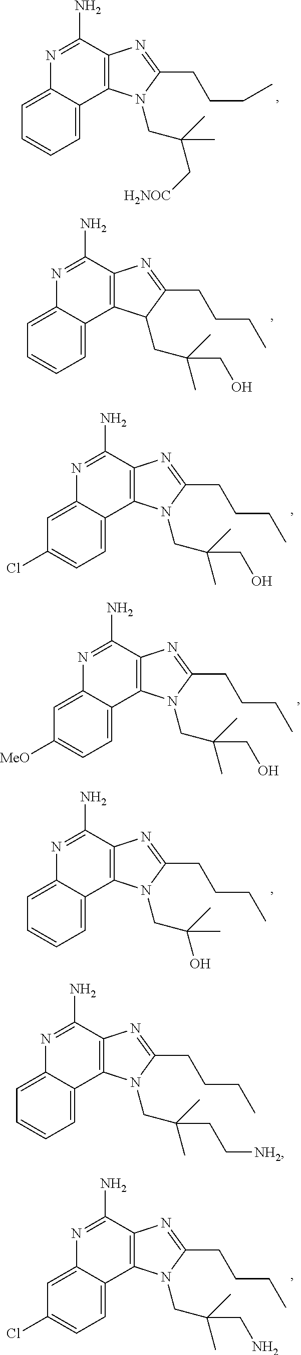

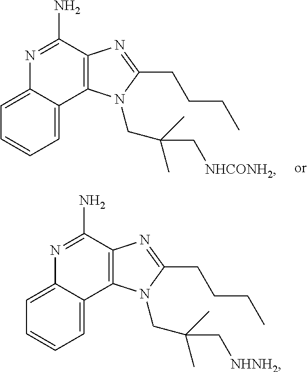

or is a pharmaceutically acceptable salt of any of the foregoing structures.

The first targeting moiety, the second targeting moiety, or both the first targeting moiety and the second targeting moiety can be a group or can comprise a group with the structure:

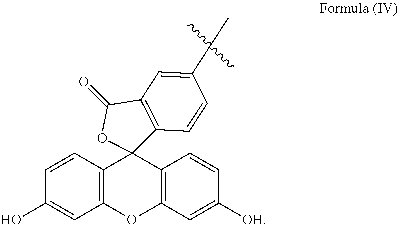 [SMILES: *c1ccc2c(c1)C(=O)OC21c2ccc(O)cc2Oc2cc(O)ccc21]

Alternatively, the first targeting moiety, the second targeting moiety, or both the first targeting moiety and the second targeting moiety can be a group or can comprise a group with the structure:

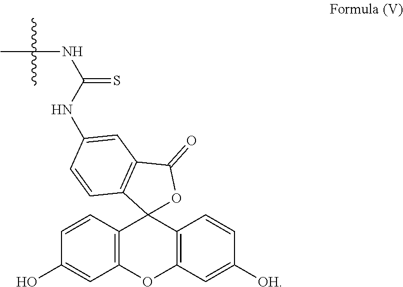 [SMILES: *NC(=S)Nc1ccc2c(c1)C(=O)OC21c2ccc(O)cc2Oc2cc(O)ccc21]

In certain embodiments, the CAR T-cell-rejuvenating conjugate can have the structure:

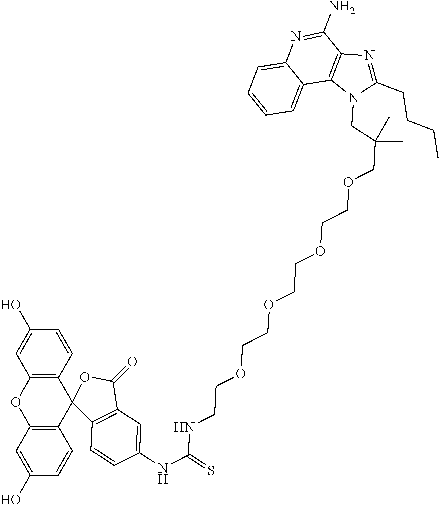 [SMILES: CCCCc1nc2c(N)nc3ccccc3c2n1CC(C)(C)COCCOCCOCCOCCNC(=S)Nc1ccc2c(c1)C(=O)OC21c2ccc(O)cc2Oc2cc(O)ccc21]

or be a pharmaceutically acceptable salt thereof. Alternatively, the CAR T-cell-rejuvenating conjugate can have the structure:

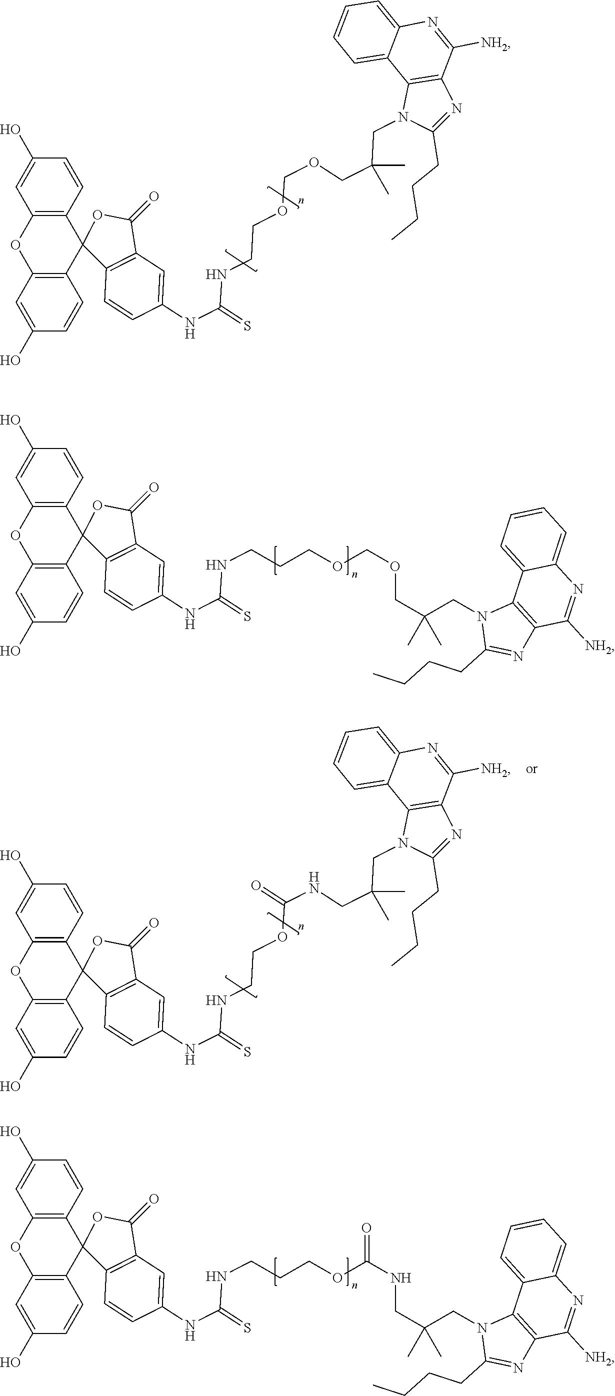

wherein n=0-200, or be a pharmaceutically acceptable salt of any of the foregoing structures.

Further alternatively, the CAR T-cell-rejuvenating conjugate can have the structure:

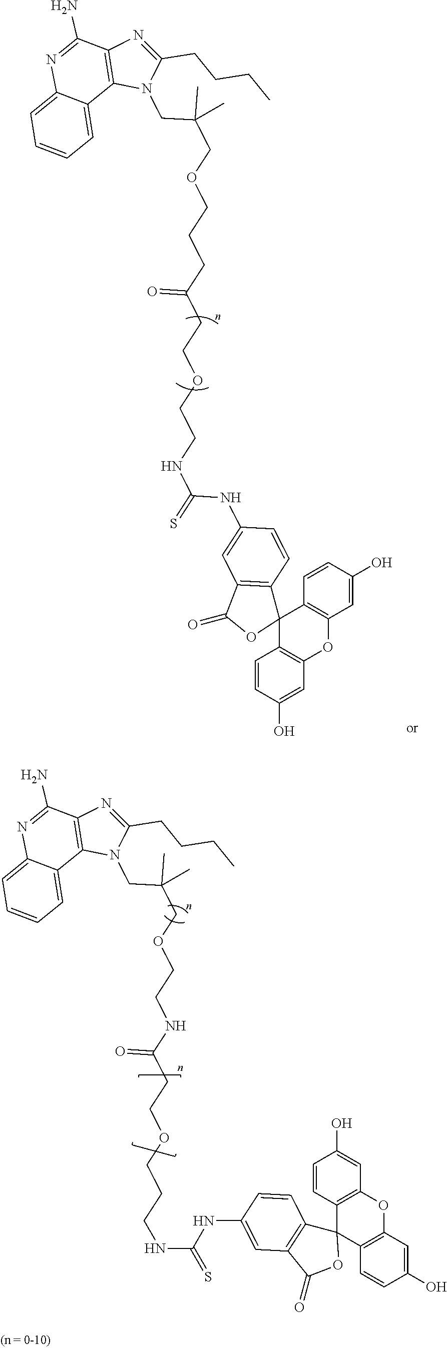

or be a pharmaceutically acceptable salt thereof. Still further, the CAR T-cell-rejuvenating conjugate can have a structure selected from:

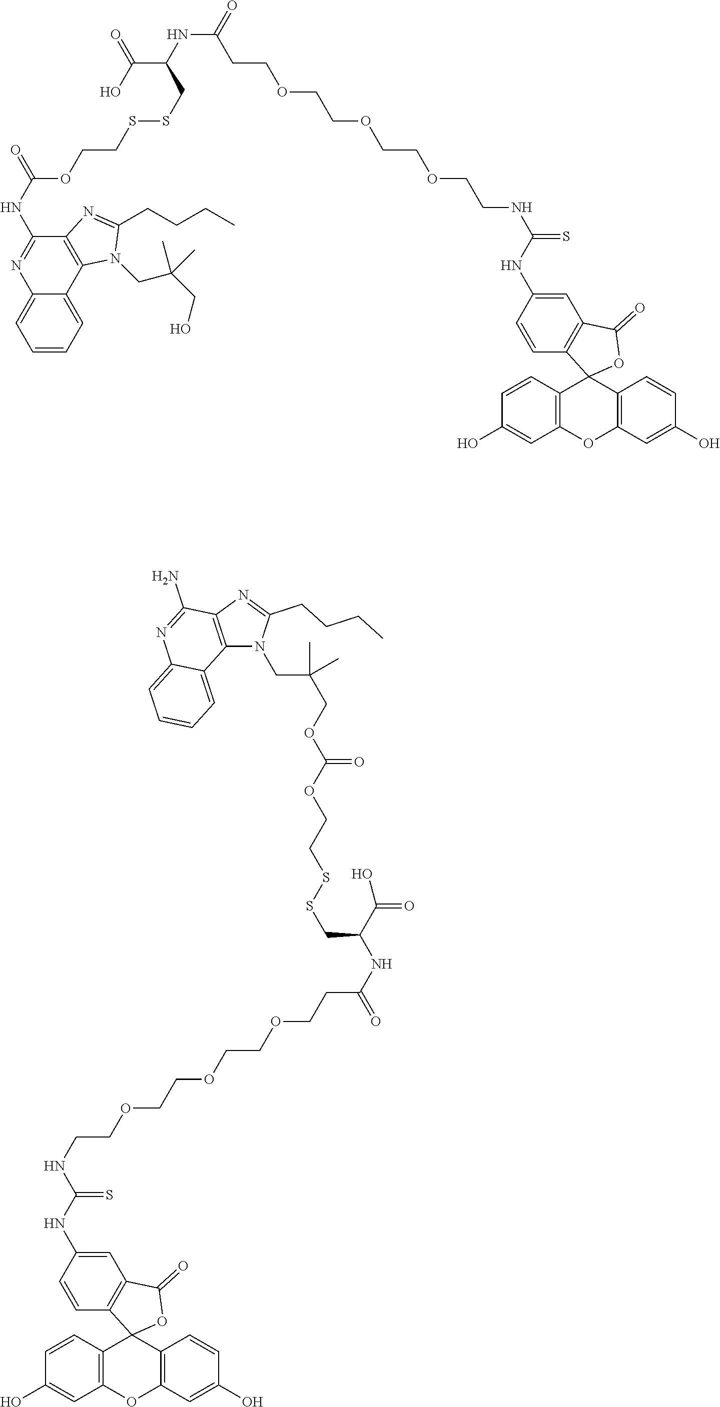 [SMILES: CCCCc1nc2c(NC(=O)OCCSSC[C@H](NC(=O)CCOCCOCCOCCNC(=S)Nc3ccc4c(c3)C(=O)OC43c4ccc(O)cc4Oc4cc(O)ccc43)C(=O)O)nc3ccccc3c2n1CC(C)(C)CO ; CCCCc1nc2c(N)nc3ccccc3c2n1CC(C)(C)COC(=O)OCCSSC[C@H](NC(=O)CCOCCOCCOCCNC(=S)Nc1ccc2c(c1)C(=O)OC21c2ccc(O)cc2Oc2cc(O)ccc21)C(=O)O]

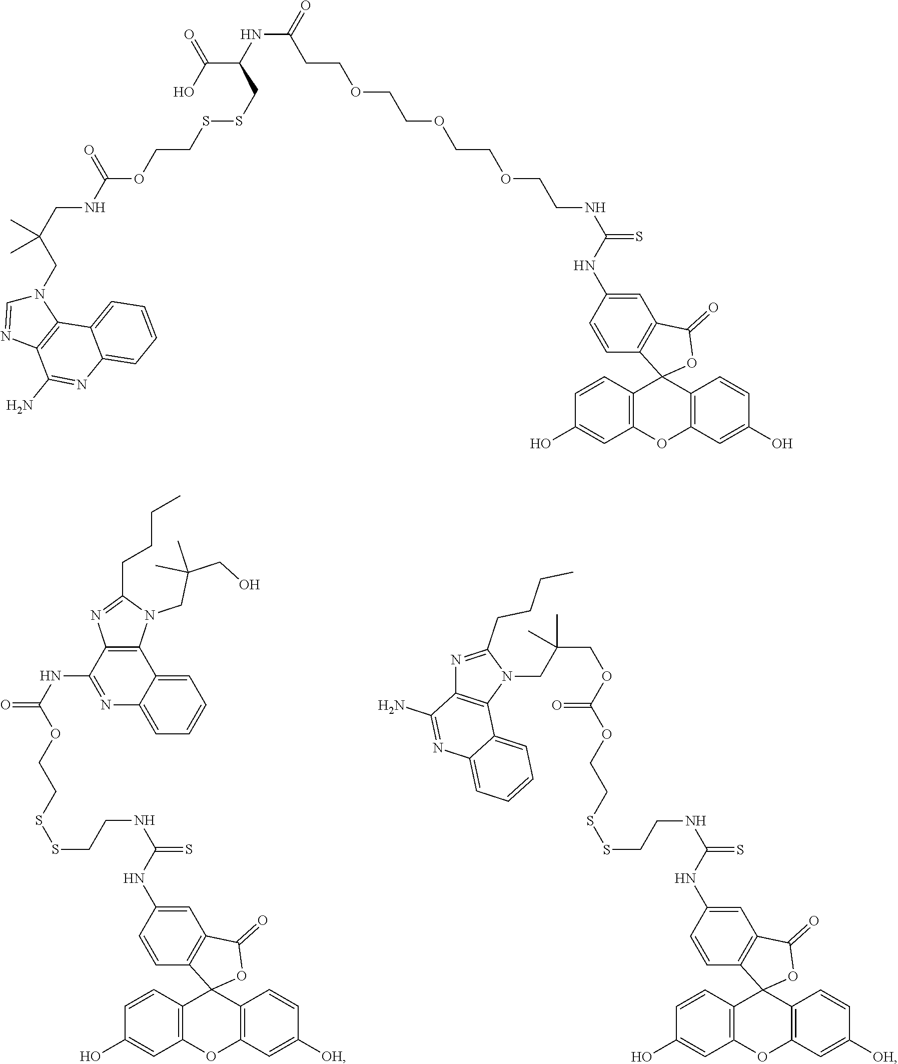 [SMILES: CC(C)(CNC(=O)OCCSSC[C@H](NC(=O)CCOCCOCCOCCNC(=S)Nc1ccc2c(c1)C(=O)OC21c2ccc(O)cc2Oc2cc(O)ccc21)C(=O)O)Cn1cnc2c(N)nc3ccccc3c21 ; CCCCc1nc2c(NC(=O)OCCSSCCNC(=S)Nc3ccc4c(c3)C(=O)OC43c4ccc(O)cc4Oc4cc(O)ccc43)nc3ccccc3c2n1CC(C)(C)CO ; CCCCc1nc2c(N)nc3ccccc3c2n1CC(C)(C)COC(=O)OCCSSCCNC(=S)Nc1ccc2c(c1)C(=O)OC21c2ccc(O)cc2Oc2cc(O)ccc21]

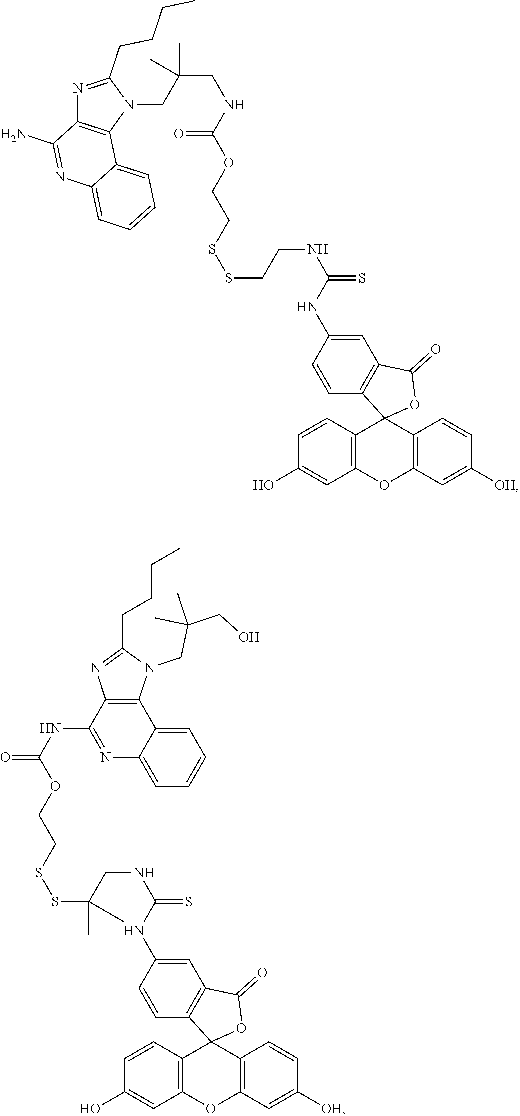 [SMILES: CCCCc1nc2c(N)nc3ccccc3c2n1CC(C)(C)CNC(=O)OCCSSCCNC(=S)Nc1ccc2c(c1)C(=O)OC21c2ccc(O)cc2Oc2cc(O)ccc21 ; CCCCc1nc2c(NC(=O)OCCSSC3(C)CNC(=S)N3c3ccc4c(c3)C(=O)OC43c4ccc(O)cc4Oc4cc(O)ccc43)nc3ccccc3c2n1CC(C)(C)CO]

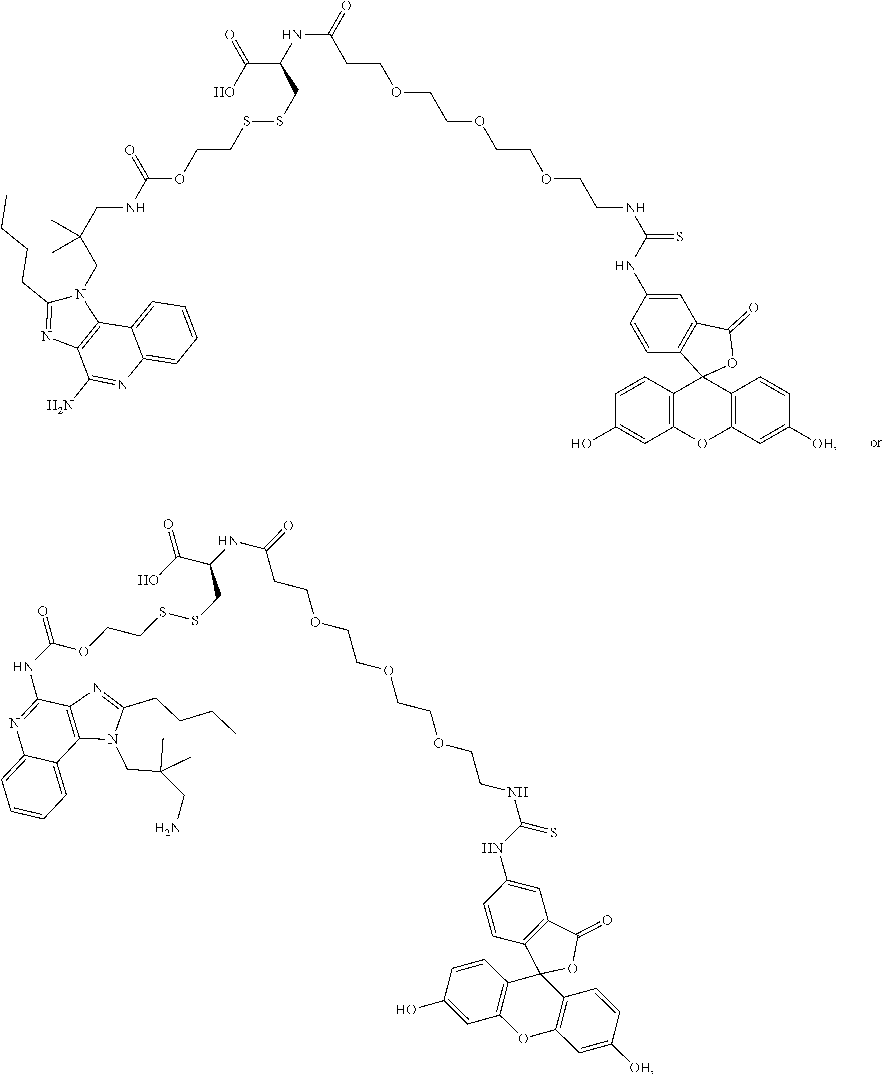 [SMILES: CCCCc1nc2c(N)nc3ccccc3c2n1CC(C)(C)CNC(=O)OCCSSC[C@H](NC(=O)CCOCCOCCOCCNC(=S)Nc1ccc2c(c1)C(=O)OC21c2ccc(O)cc2Oc2cc(O)ccc21)C(=O)O ; CCCCc1nc2c(NC(=O)OCCSSC[C@H](NC(=O)CCOCCOCCOCCNC(=S)Nc3ccc4c(c3)C(=O)OC43c4ccc(O)cc4Oc4cc(O)ccc43)C(=O)O)nc3ccccc3c2n1CC(C)(C)CN]

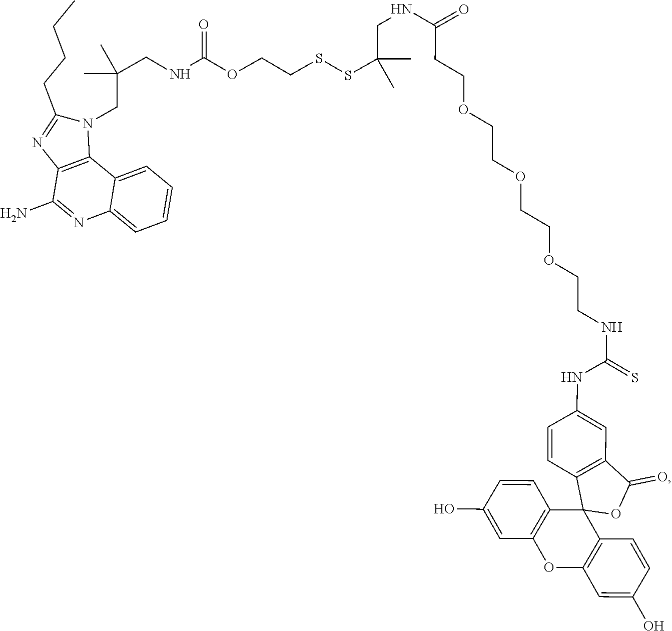 [SMILES: CCCCc1nc2c(N)nc3ccccc3c2n1CC(C)(C)CNC(=O)OCCSSC(C)(C)CNC(=O)CCOCCOCCOCCNC(=S)Nc1ccc2c(c1)C(=O)OC21c2ccc(O)cc2Oc2cc(O)ccc21]

or be a pharmaceutically acceptable salt of any of the foregoing structures. In certain embodiments, the CAR T-cell-rejuvenating conjugate can have a structure selected from:

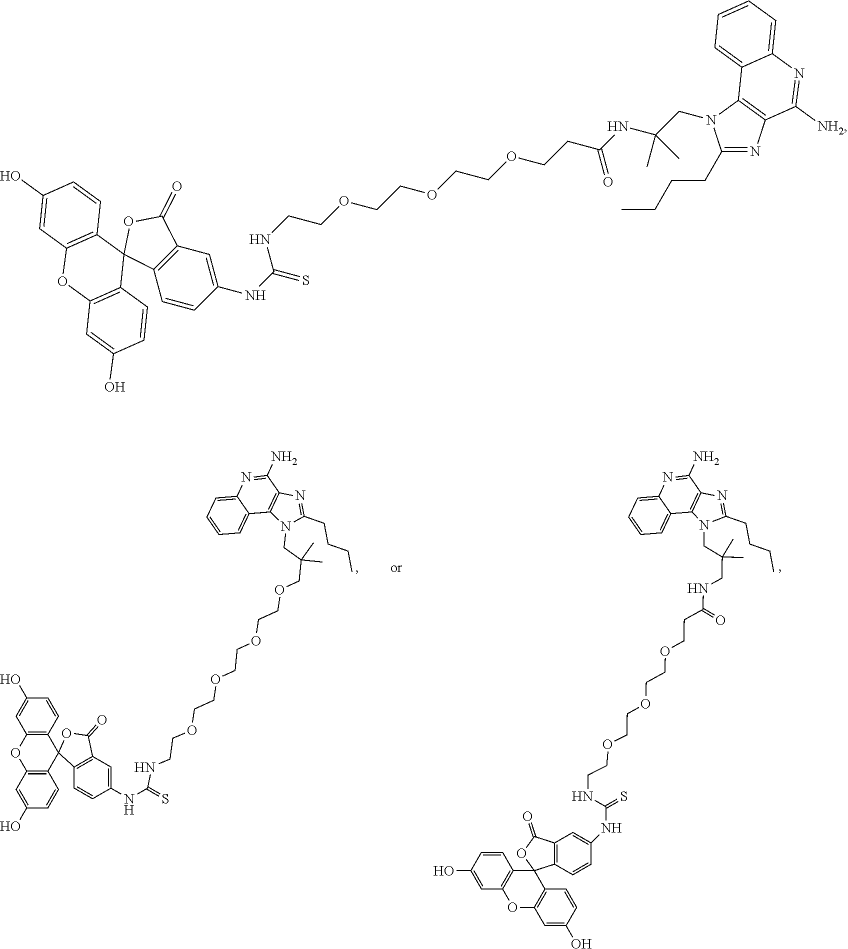 [SMILES: CCCCc1nc2c(N)nc3ccccc3c2n1CC(C)(C)NC(=O)CCOCCOCCOCCNC(=S)Nc1ccc2c(c1)C(=O)OC21c2ccc(O)cc2Oc2cc(O)ccc21 ; CCCCc1nc2c(N)nc3ccccc3c2n1CC(C)(C)COCCOCCOCCOCCNC(=S)Nc1ccc2c(c1)C(=O)OC21c2ccc(O)cc2Oc2cc(O)ccc21 ; CCCCc1nc2c(N)nc3ccccc3c2n1CC(C)(C)CNC(=O)CCOCCOCCOCCNC(=S)Nc1ccc2c(c1)C(=O)OC21c2ccc(O)cc2Oc2cc(O)ccc21]

or be a pharmaceutically acceptable salt of any of the foregoing structures. In certain embodiments, the CAR T-cell-rejuvenating conjugate can have a structure selected from:

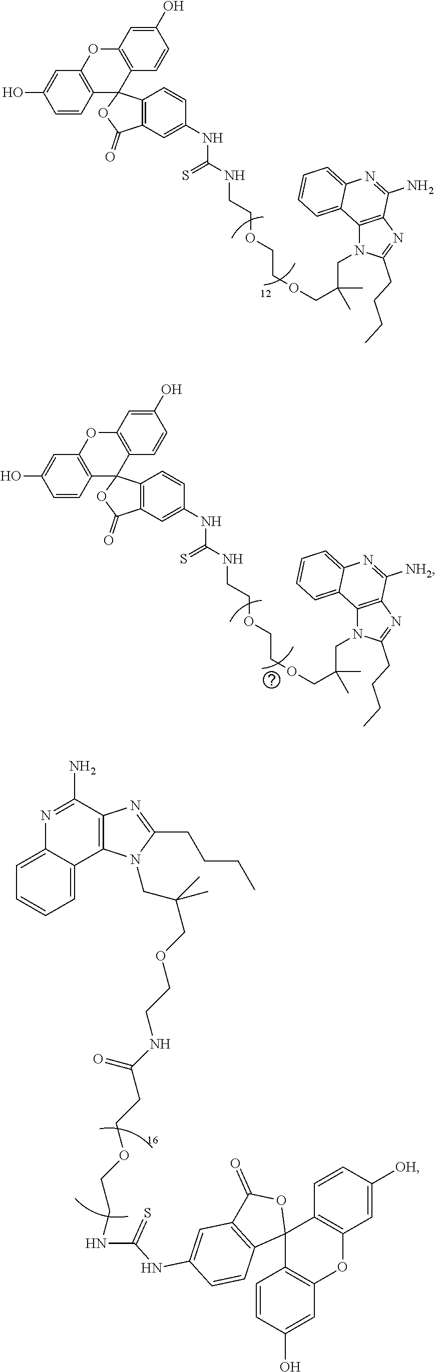

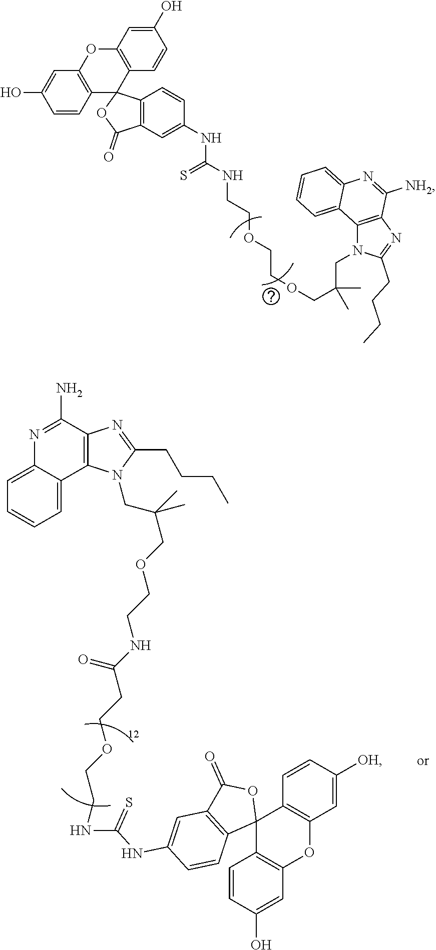

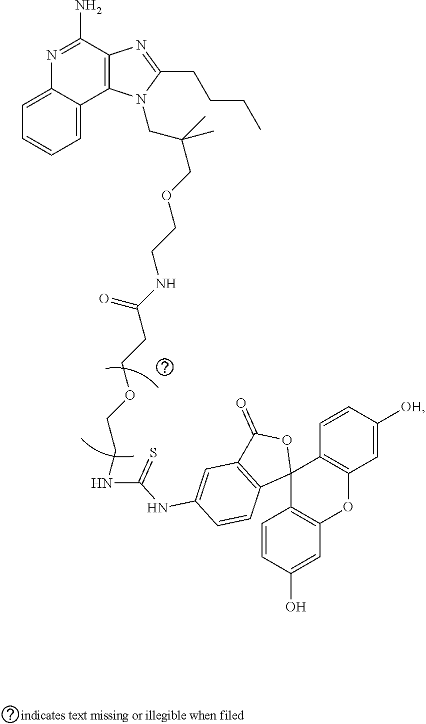

or be a pharmaceutically acceptable salt of any of the foregoing structures. In certain embodiments, the CAR T-cell-rejuvenating conjugate can have a structure selected from:

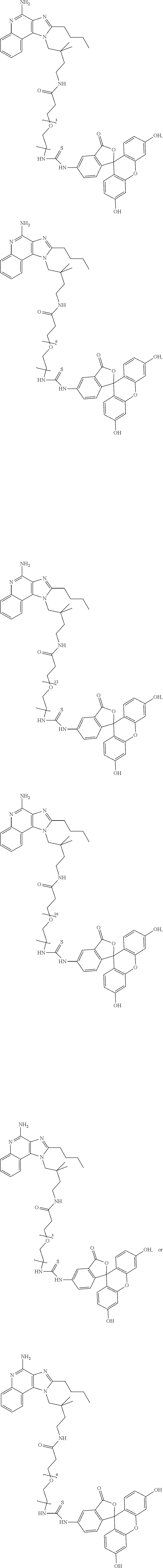

or be a pharmaceutically acceptable salt of any of the foregoing structures.

The ligand that binds a cancer cell with specificity can be selected from the group consisting of a folate, 5-methyltetrahydrofolate, a 2-[3-(1,3-dicarboxypropyl)ureido]pentanedioic acid (DUPA) ligand, a neurokinin 1 receptor (NK-1R) ligand, a carbonic anhydrase IX (CAIX) ligand, a ligand of gamma glutamyl transpeptidase, a ligand of luteinizing hormone releasing hormone (LHRHR), a ligand of CD73, a ligand of fibroblast activation protein, a ligand of heat shock protein (HSP), a ligand of glucose transporter 1 (glut-1), a ligand of a natural killer group 2D receptor (NKG2D) ligand, and a cholecystokinin B receptor (CCKBR or CCK2) ligand.

The first targeting moiety and the second targeting moiety can be independently selected from the group consisting of 2,4-dinitrophenyl (DNP), L-rhamnose, tacrolimus (FK506), 2,4,6-trinitrophenol (TNP), biotin, rapamycin, digoxigenin, folate, 5-methyl tetrahydrofolate, fluorescein, fluorescein isothiocyanate (FITC), NHS-fluorescein, pentafluorophenyl ester, tetrafluorophenyl ester, knottin, centyrin, and DARPin.

In certain embodiments, the first targeting moiety and the second targeting moiety are independently selected from the group consisting of DNP, FK506, TNP, biotin, rapamycin, digoxigenin, folate, 5-methyl tetrahydrofolate, fluorescein, FITC, NHS-fluorescein, pentafluorophenyl ester, tetrafluorophenyl ester, knottin, centyrin, and DARPin; and the ligand that binds a cancer cell with specificity is selected from the group consisting of a folate, a DUPA ligand, a NK-1R ligand, a CAIX ligand, a ligand of gamma glutamyl transpeptidase, a ligand of LHRHR, a ligand of CD73, a ligand of HSP, a ligand of glut-1, a ligand of fibroblast activation protein, a ligand of a NKG2D ligand and a CCKBR or CCK2 ligand.

The first linker and the second linker can be independently releasable or non-releasable. The first linker and the second linker can independently comprise C₁-C₂₀ alkyl, alkylene, heteroalkylene, —O-alkynylene, alkenylene, acyl, aryl, heteroaryl, amide, oxime, ether, ester, triazole, —S—S, —CO—O—(CH₂)ₙ—S—S— (where n=2-6), —O—CO—O—(CH₂)ₙ—S—S— (where n=2-6), —S—CO—O—(CH₂)ₙ—S—S— (where n=2-6), —NH—CO—O—(CH₂)ₙ—S—S— (where n=2-6), carboxylate, carbonate, carbamate, urea, thiourea, polyethylene glycol (PEG) (e.g., PEGₙ, where n=1-200), polyproline, oligo-(4-piperidine) carboxylic acid, oligo piperidine, amino acid (e.g., hydrophilic amino acid), peptide, saccharo-peptide, sugar, peptidoglycan (e.g., an unnatural peptidoglycan), a polyvinylpyrrolidone, pluronic F-127, or any combination of two or more of the foregoing. The first linker, the second linker, or both the first linker and the second linker can comprise PEG.

Also provided is a CAR T-cell-rejuvenating conjugate comprising an agonist of TLR 7, TLR 8, or TLR7/8 conjugated via a linker with a targeting moiety that binds a CAR of a CAR T-cell with specificity, wherein the CAR T-cell-rejuvenating conjugate has the structure:

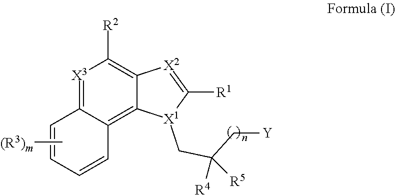

or is a pharmaceutically acceptable salt thereof, wherein:
   R¹ is an acyclic or cyclic alkyl, which is optionally substituted with one or more substituents independently selected from an alkyl, halo, heteroalkyl, alkoxy, and cycloalkyl;
   R² is —NR^{2x}R^{2y}, H, —OR^{z}, —SO₂N(R^{z})₂, or N₃, wherein:
      R^{2x} and R^{2y} are each independently hydrogen, —N(R^{z})₂, —CON(R^{z})₂, —C(R^{z})₂—N(R^{z})₂, —CS—N(R^{z})₂, or an alkyl, which is optionally substituted with one or more substituents independently selected from oxo, halo, alkyl, heteroalkyl, alkoxy, and cycloalkyl;
      each R^{Z} is independently H or alkyl that is optionally substituted; or
      R^{2x} and R^{2y} are taken together to form a 3-10 membered mono- or bicyclic heterocycloalkyl, which is optionally substituted;
   each R³ is independently H, halo, —N₃, —CN, —NO₂, alkyl, heteroalkyl, cycloalkyl, heterocycloalkyl, alkoxy, aryl, heteroaryl, heterocycloalkyl, amino, hydroxy, carbonyl, or thiol, wherein the alkyl, heteroalkyl, cycloalkyl, heterocycloalkyl, or alkoxy that is optionally substituted;
   R⁴ and R⁵ are each independently alkyl, alkoxy, halo, or cycloalkyl, wherein the alkyl, alkoxy, or cycloalkyl that is optionally substituted;
   X¹, X², and X³ are independently N or CR^{q}, wherein each R^{q} is independently H, halo, or optionally substituted alkyl;
   Z is G-L-, wherein L is a linker and G is a targeting moiety;
   n in Formula (I) is 1-6; and
   m in Formula (II) is 0-4; or
   wherein the CAR T-cell-rejuvenating conjugate has the structure:

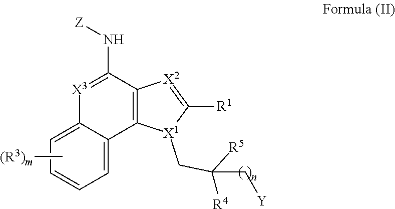

or is a pharmaceutically acceptable salt thereof, wherein:
   R¹, R³, R⁴, and R⁵ are each independently H, alkyl, alkoxyl, alkenyl, alkynyl, alicyclic, aryl, biaryl, halo, heteroaryl, —COR^{2x},

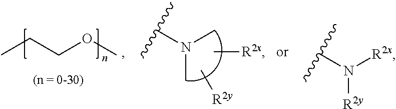

wherein each of R^{2x} and R^{2y} is independently selected from the group consisting of H, —OH, —CH₂—OH, —NH₂, —CH₂—NH₂. —COOMe, —COOH, —CONH₂, —COCH₃, alkyl, alkenyl, alkynyl, alicyclic, aryl, biaryl, and heteroaryl;
   Z is G-L-, wherein L is a linker and G is a targeting moiety;
   X¹, X², and X³ are independently CR^{q} or N, wherein each R^{q} is independently H, halo, or optionally substituted alkyl;
   Y is H, —OR^{z}, —NR^{2x}R^{2y}, —SR^{z}, —SOR^{z}, —SO₃R^{z}, —N₃, —COR^{z}, —COOR^{z}, —CON(R^{z})₂, —COSR^{z}, —SO₂N(R^{z})₂, or —CON(R^{z})₂, wherein:
      R^{2x} and R^{2y} are each independently H, —N(R^{z})₂, —CON(R^{z})₂, —C(R^{z})₂—N(R^{z})₂, —CS—N(R^{z})₂, or optionally substituted alkyl (e.g., optionally substituted with one or more substituent, each substituent independently being oxo, halogen, alkyl, heteroalkyl, alkoxy, or cycloalkyl);
      each R^{z} is independently hydrogen, or optionally substituted alkyl; or
      R^{2x} and R^{2y} are taken together to form an optionally substituted heterocycloalkyl (e.g., wherein the optionally substituted heterocycloalkyl is a mono- or bicyclic heterocycloalkyl and/or wherein the optionally substituted heterocycloalkyl is a 3-10 membered heterocycloalkyl); and
   n in Formula (I) is 0-30; or
   wherein the CAR T-cell-rejuvenating conjugate has the structure:

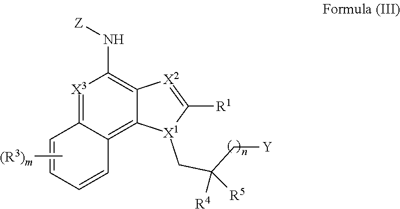

or is a pharmaceutically acceptable salt thereof, wherein:
   R¹ is an acyclic or cyclic alkyl, which is optionally substituted with one or more substituents independently selected from halo, alkyl, heteroalkyl, alkoxy, and cycloalkyl;
   Y is H, —OR^{z}, —NR^{2x}R^{2y}, —SR^{z}, —SOR^{z}, —SO₃R^{z}, —N₃, —COR^{z}, —COOR^{z}, —CONR^{z}₂, —COSR^{z}, —SO₂N(R^{z})₂, or —CON(R^{z})₂, wherein:
      each R^{z} is independently H, or optionally substituted alkyl;
      R^{2x} and R^{2y} are each independently H, —N(R^{z})₂, —CON(R^{z})₂, —C(R^{z})₂—N(R^{z})₂, —CS—N(R^{Z})₂, or an alkyl that is optionally substituted with one or more substituents independently selected from oxo, halo, alkyl, heteroalkyl, alkoxy, and cycloalkyl; or
      R^{2x} and R^{2y} are taken together to form a 3-10 membered mono- or bicyclic heterocycloalkyl, which is optionally substituted;
   each R³ is independently halo, —N₃, —CN, —NO₂, alkyl, heteroalkyl, cycloalkyl, heterocycloalkyl, alkoxy, aryl, heteroaryl, heterocycloalkyl, amino, hydroxy, carbonyl, or thiol, wherein the alkyl, alkoxy, heteroalkyl, cycloalkyl, or heterocycloalkyl is optionally substituted;
   R⁴ and R⁵ are each independently alkyl, alkoxy, halogen, or cycloalkyl, wherein the alkyl, alkoxy, or cycloalkyl is optionally substituted;
   X¹, X², and X³ are independently CR^{q} or N, wherein each R is independently H, halogen, or optionally substituted alkyl;
   Z is L-G, wherein L is a linker and G is a targeting moiety;
   n of Formula (III) is 0-30; and
   m of Formula (III) is 0-4;
   wherein G in Formula (I), Formula (II) or Formula (III) is a group or comprises a group with the structure:

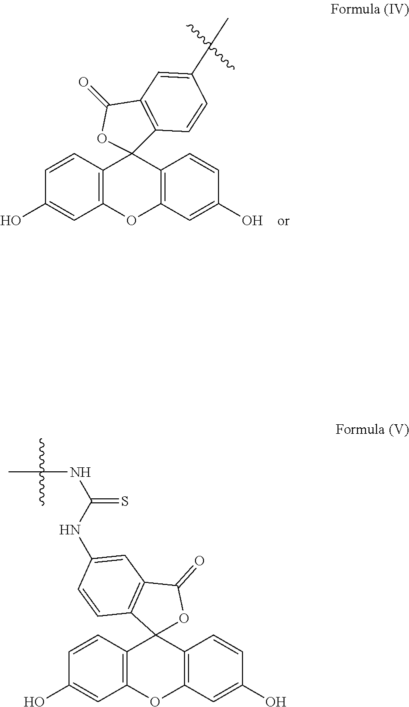

or is a pharmaceutically acceptable salt thereof. In various embodiments, each of X¹, X², and X³ of Formula (I), Formula (II) or Formula (III) can be nitrogen (N).

The targeting moiety of the CAR T-cell-rejuvenating conjugate can be selected from the group consisting of DNP, L-rhamnose, FK506, TNP, biotin, rapamycin, digoxigenin, folate, 5-methyl tetrahydrofolate, fluorescein, FITC, NHS-fluorescein, pentafluorophenyl ester, tetrafluorophenyl ester, knottin, centyrin, and DARPin.

The linker of the CAR T-cell-rejuvenating conjugate can be releasable. The linker of the CAR T-cell-rejuvenating conjugate can be nonreleasable. The linker can comprise C₁-C₂₀ alkyl, alkylene, heteroalkylene, —O-alkynylene, alkenylene, acyl, aryl, heteroaryl, amide, oxime, ether, ester, triazole, —S—S, —CO—O—(CH₂)ₙ—S—S— (where n=2-6), —O—CO—O—(CH₂)ₙ—S—S— (where n=2-6), —S—CO—O—(CH₂)ₙ—S—S— (where n=2-6). —NH—CO—O—(CH₂)ₙ—S—S— (where n=2-6), carboxylate, carbonate, carbamate, urea, thiourea, PEG (e.g., PEGₙ, where n=1-200), polyproline, oligo-(4-piperidine) carboxylic acid, oligo piperidine, amino acid (e.g., hydrophilic amino acid), peptide, saccharo-peptide, sugar, peptidoglycan (e.g., an unnatural peptidoglycan), a polyvinylpyrrolidone, pluronic F-127, or any combination of two or more of the foregoing. The linker can comprise PEG.

The CAR T-cell-rejuvenating conjugate can have the structure:

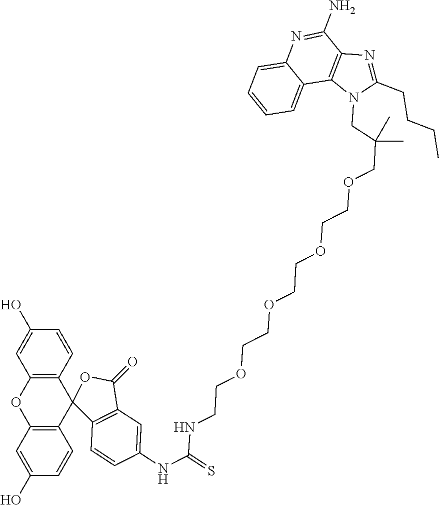 [SMILES: CCCCc1nc2c(N)nc3ccccc3c2n1CC(C)(C)COCCOCCOCCOCCNC(=S)Nc1ccc2c(c1)C(=O)OC21c2ccc(O)cc2Oc2cc(O)ccc21]

or be a pharmaceutically acceptable salt thereof. The CAR T-cell-rejuvenating conjugate can have a structure selected from:

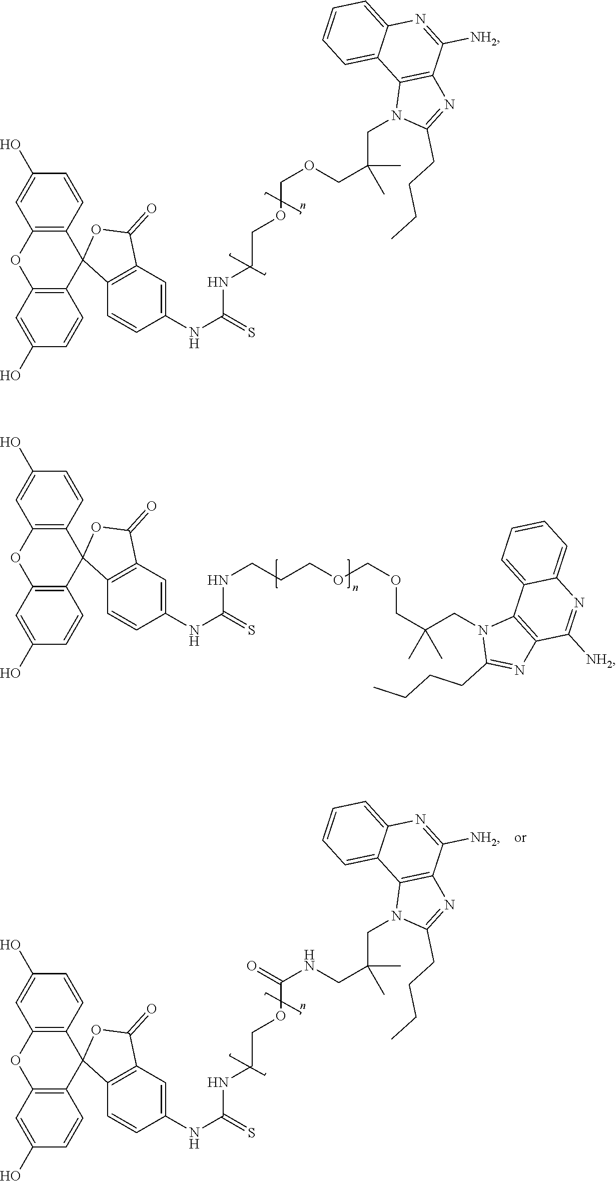

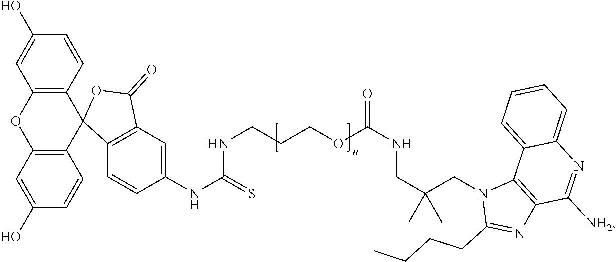

wherein n=0-200, or be a pharmaceutically acceptable salt of any of the foregoing structures. The CAR T-cell-rejuvenating conjugate can have a structure selected from:

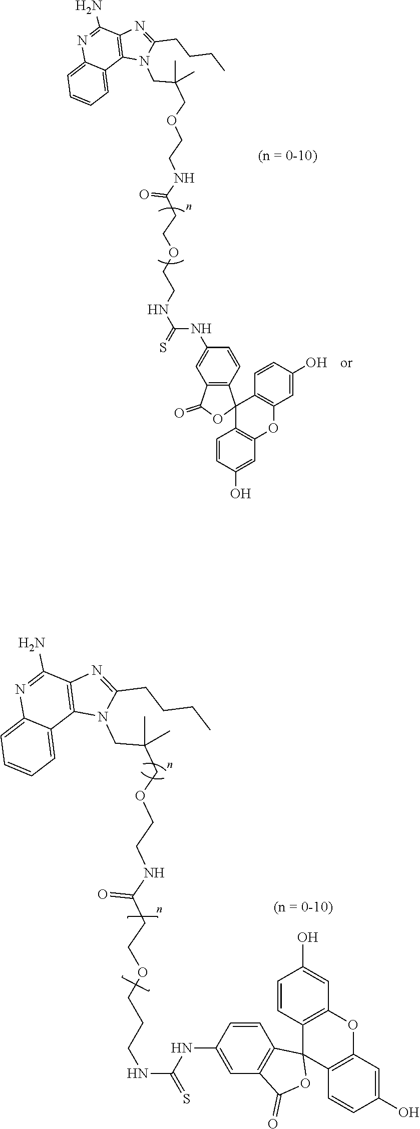

or be a pharmaceutically acceptable salt thereof. Still further alternatively, the CAR T-cell-rejuvenating conjugate can have a structure selected from:

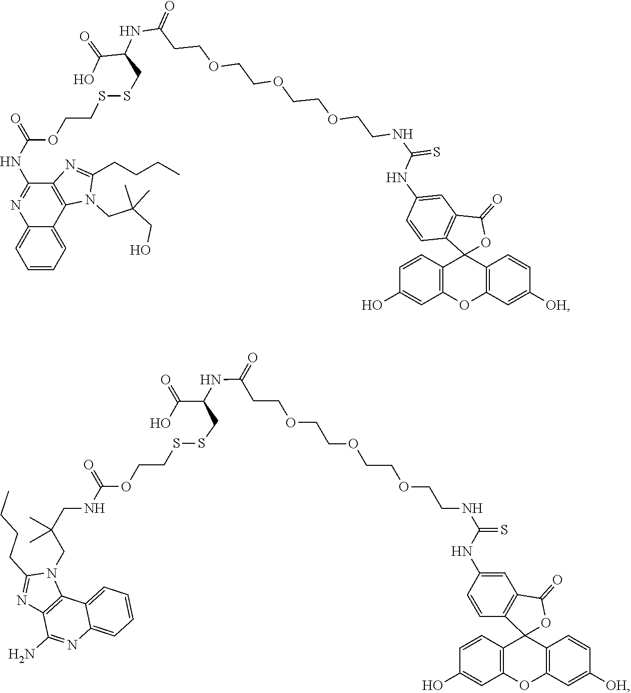 [SMILES: CCCCc1nc2c(NC(=O)OCCSSC[C@H](NC(=O)CCOCCOCCOCCNC(=S)Nc3ccc4c(c3)C(=O)OC43c4ccc(O)cc4Oc4cc(O)ccc43)C(=O)O)nc3ccccc3c2n1CC(C)(C)CO ; CCCCc1nc2c(N)nc3ccccc3c2n1CC(C)(C)CNC(=O)OCCSSC[C@H](NC(=O)CCOCCOCCOCCNC(=S)Nc1ccc2c(c1)C(=O)OC21c2ccc(O)cc2Oc2cc(O)ccc21)C(=O)O]

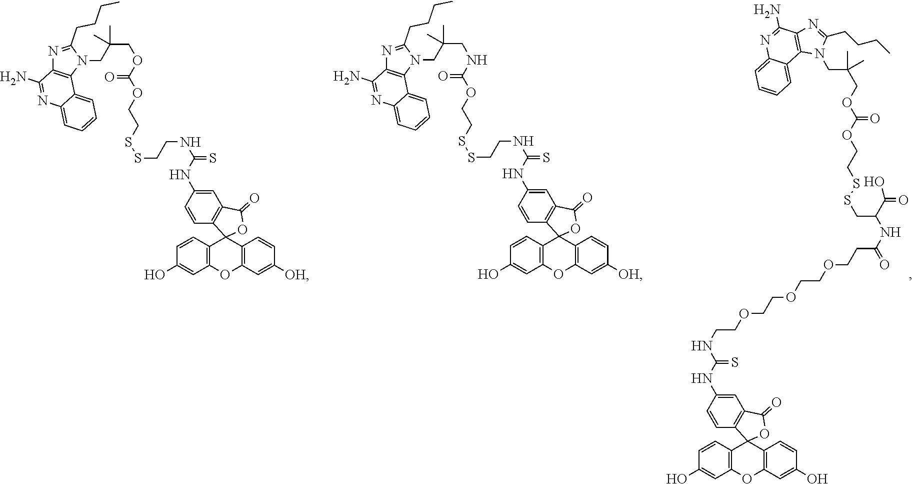 [SMILES: CCCCc1nc2c(N)nc3ccccc3c2n1CC(C)(C)COC(=O)OCCSSCCNC(=S)Nc1ccc2c(c1)C(=O)OC21c2ccc(O)cc2Oc2cc(O)ccc21 ; CCCCc1nc2c(N)nc3ccccc3c2n1CC(C)(C)CNC(=O)OCCSSCCNC(=S)Nc1ccc2c(c1)C(=O)OC21c2ccc(O)cc2Oc2cc(O)ccc21 ; CCCCc1nc2c(N)nc3ccccc3c2n1CC(C)(C)COC(=O)OCCSSCC(NC(=O)CCOCCOCCOCCNC(=S)Nc1ccc2c(c1)C(=O)OC21c2ccc(O)cc2Oc2cc(O)ccc21)C(=O)O ; ","]

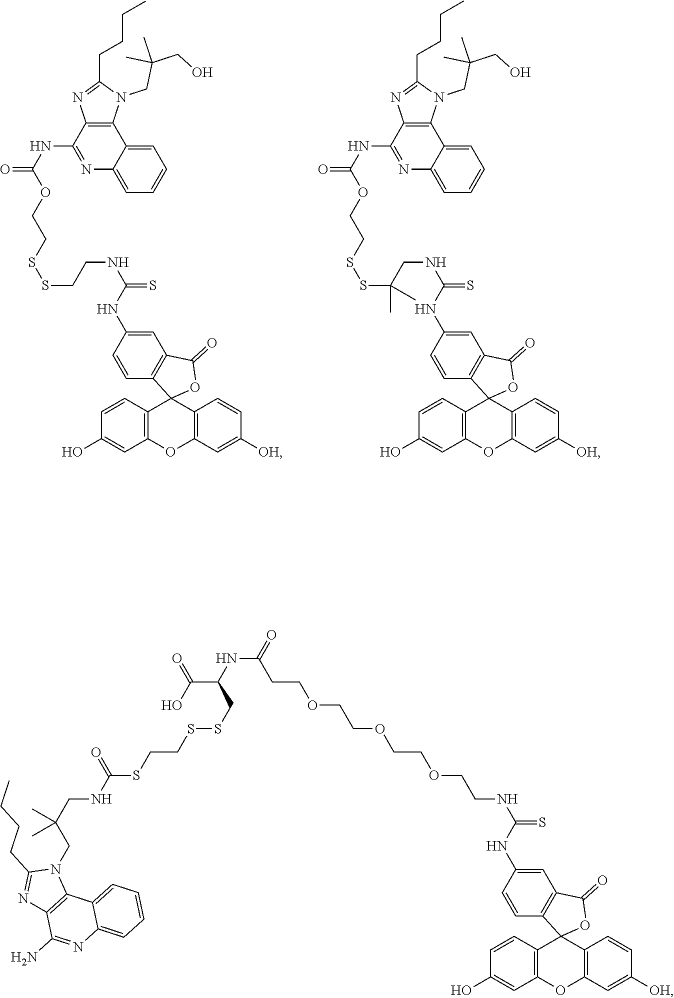 [SMILES: CCCCc1nc2c(NC(=O)OCCSSCCNC(=S)Nc3ccc4c(c3)C(=O)OC43c4ccc(O)cc4Oc4cc(O)ccc43)nc3ccccc3c2n1CC(C)(C)CO ; CCCCc1nc2c(NC(=O)OCCSSC3(C)CNC(=S)N3c3ccc4c(c3)C(=O)OC43c4ccc(O)cc4Oc4cc(O)ccc43)nc3ccccc3c2n1CC(C)(C)CO ; CCCCc1nc2c(N)nc3ccccc3c2n1CC(C)(C)CNC(=O)SCCSSC[C@H](NC(=O)CCOCCOCCOCCNC(=S)Nc1ccc2c(c1)C(=O)OC21c2ccc(O)cc2Oc2cc(O)ccc21)C(=O)O]

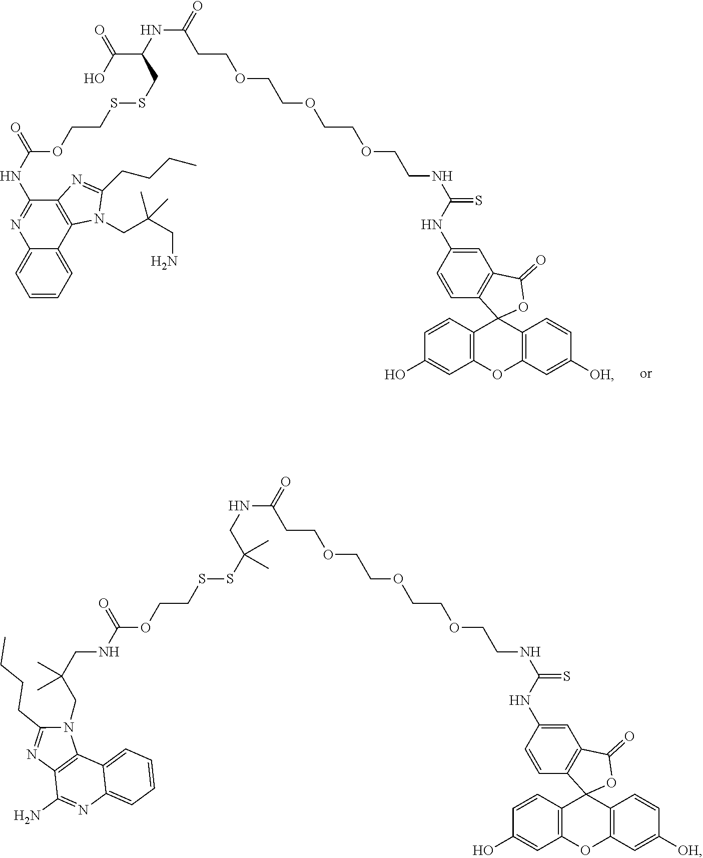 [SMILES: CCCCc1nc2c(NC(=O)OCCSSC[C@H](NC(=O)CCOCCOCCOCCNC(=S)Nc3ccc4c(c3)C(=O)OC43c4ccc(O)cc4Oc4cc(O)ccc43)C(=O)O)nc3ccccc3c2n1CC(C)(C)CN ; CCCCc1nc2c(N)nc3ccccc3c2n1CC(C)(C)CNC(=O)OCCSSC(C)(C)CNC(=O)CCOCCOCCOCCNC(=S)Nc1ccc2c(c1)C(=O)OC21c2ccc(O)cc2Oc2cc(O)ccc21]

or be a pharmaceutically acceptable salt of any of the foregoing structures.

In certain embodiments, the CAR T-cell-rejuvenating conjugate can have a structure selected from:

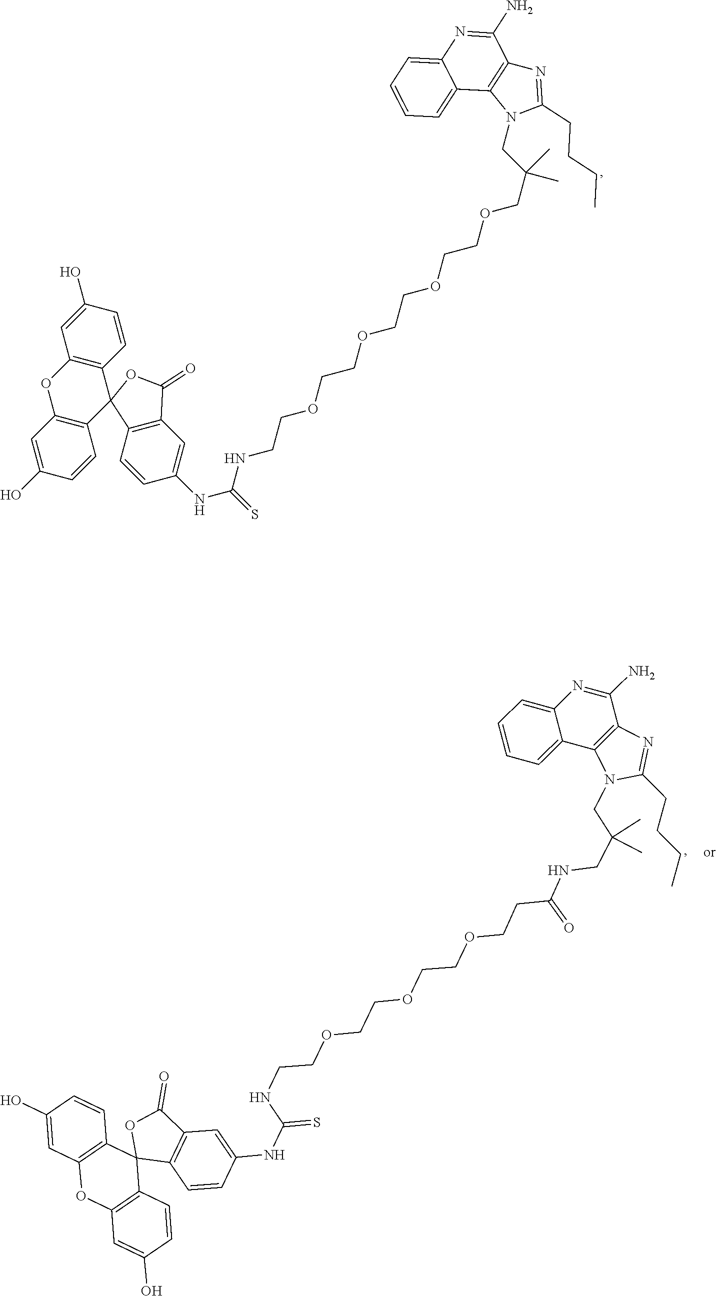 [SMILES: CCCCc1nc2c(N)nc3ccccc3c2n1CC(C)(C)COCCOCCOCCOCCNC(=S)Nc1ccc2c(c1)C(=O)OC21c2ccc(O)cc2Oc2cc(O)ccc21 ; CCCCc1nc2c(N)nc3ccccc3c2n1CC(C)(C)CNC(=O)CCOCCOCCOCCNC(=S)Nc1ccc2c(c1)C(=O)OC21c2ccc(O)cc2Oc2cc(O)ccc21]

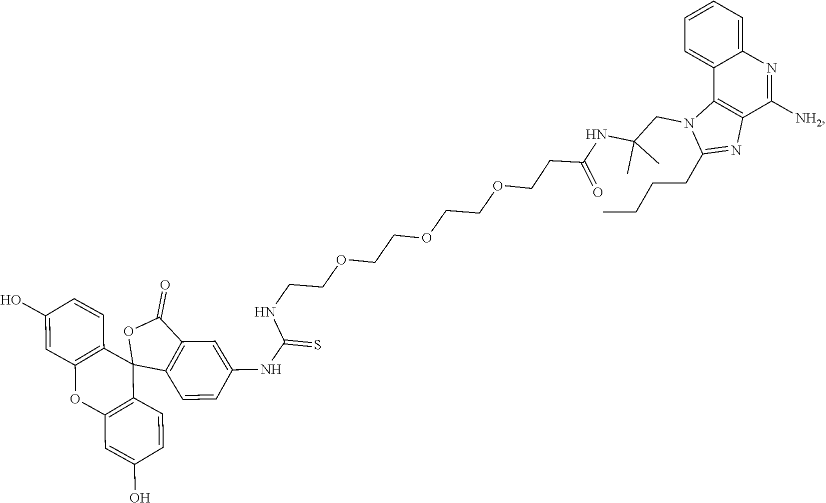 [SMILES: CCCCc1nc2c(N)nc3ccccc3c2n1CC(C)(C)NC(=O)CCOCCOCCOCCNC(=S)Nc1ccc2c(c1)C(=O)OC21c2ccc(O)cc2Oc2cc(O)ccc21]

or is a pharmaceutically acceptable salt of any of the foregoing structures. Other structures of CAR T-cell-rejuvenating conjugates include structures selected from:

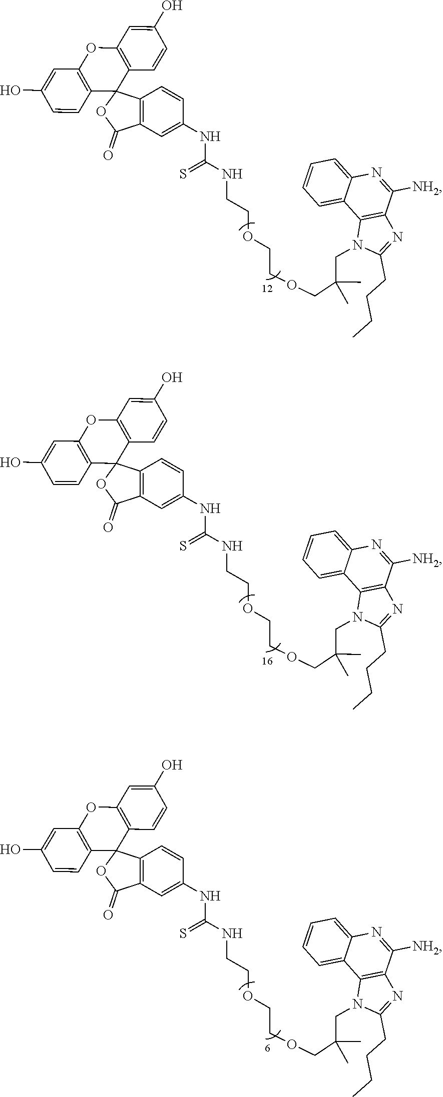

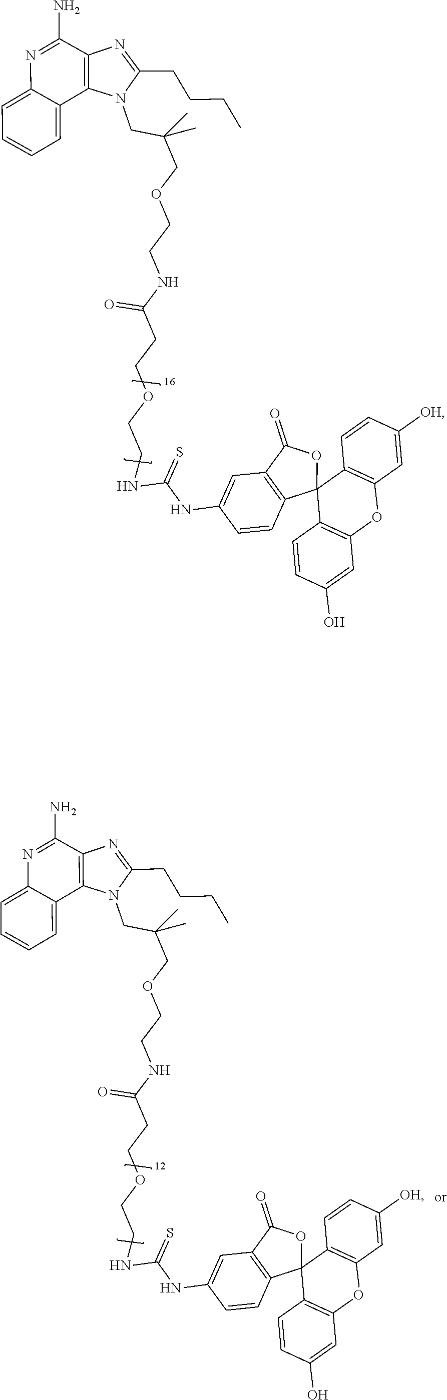

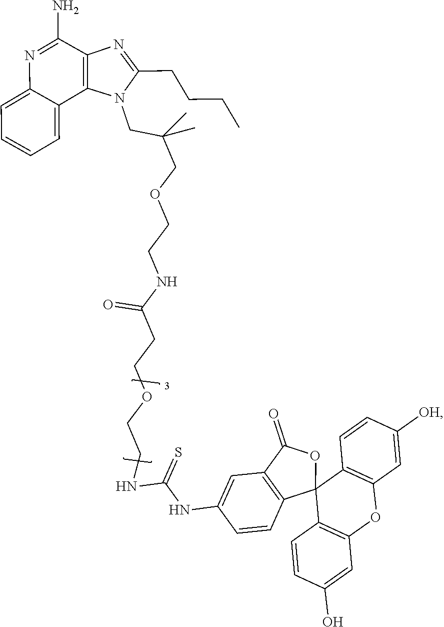

or can be a pharmaceutically acceptable salt of any of the foregoing structures. Still further, in certain embodiments, the CAR T-cell rejuvenating conjugate can comprise a structure selected from:

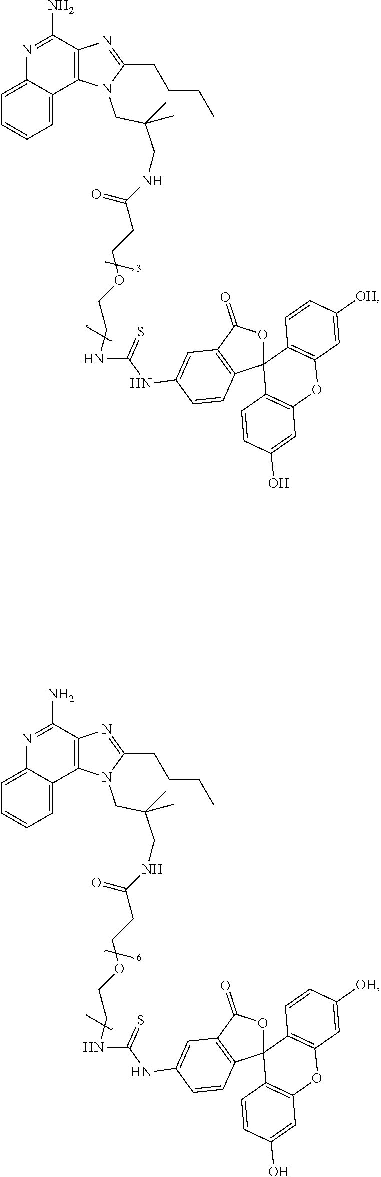

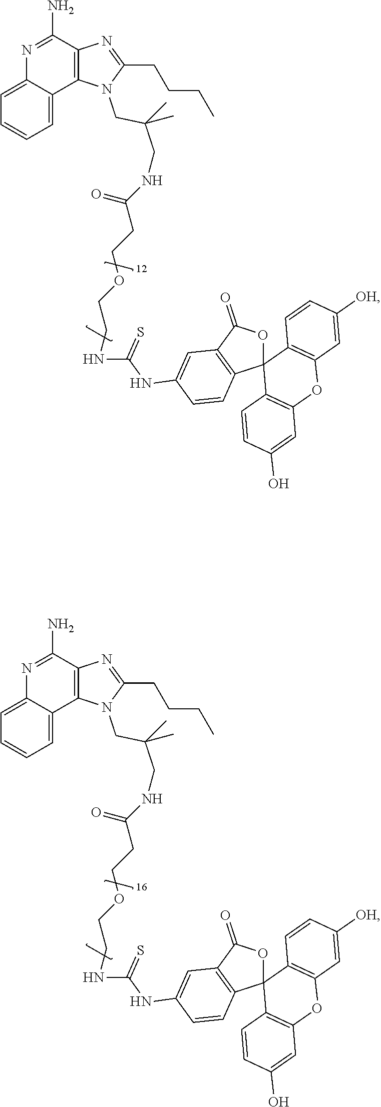

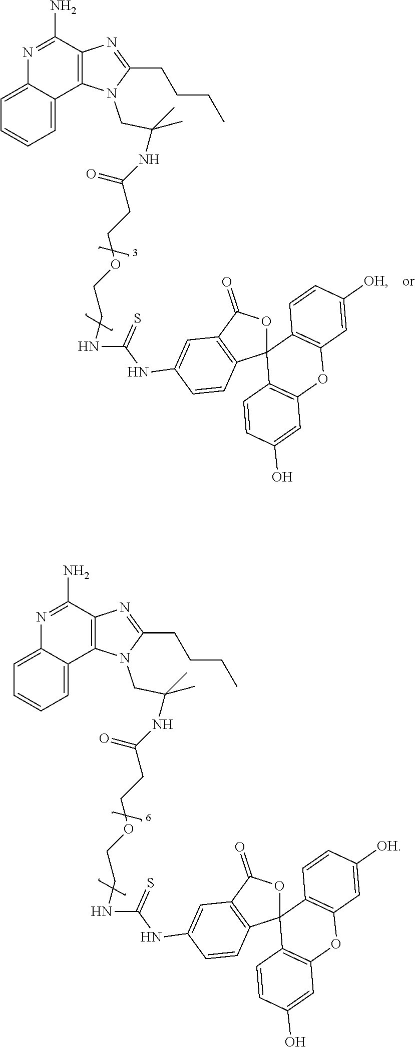

or can be a pharmaceutically acceptable salt of any of the foregoing structures.

A pharmaceutical composition is also provided. The composition can comprise the CAR T-cell-rejuvenating conjugate and a pharmaceutically acceptable carrier.

### DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIGS. 1A and 1B are graphical representations of a chimeric antigen receptor (CAR) T-cell rejuvenation strategy. FIG. 1A shows the cell-killing effect of CAR T-cells in the presence of an adaptor compound. FIG. 1B shows the rejuvenation of CAR T-cells in the presence of a targeted rejuvenating compound.

FIGS. 2A and 2B are graphical representations of an embodiment of a CAR T-cell rejuvenation strategy employing anti-fluorescein CAR and a fluorescein-Toll-like receptor (TLR) 7a conjugate to activate CAR T-cells selectively in vivo. FIG. 2A shows that, upon administration of a fluorescein-folate conjugate (an embodiment of a “cancer-killing” conjugate) to a subject having a folate receptor-expressing tumor, the “cancer-killing” conjugate formed abridge between the anti-fluorescein CAR T-cell (i.e., a CAR T-cell with an anti-fluorescein cell-surface receptor) and the folate receptor-expressing cancer cell, and promoted the killing of the cancer cells and proliferation of the CAR T-cells. FIG. 2B shows that, upon administration of a fluorescein-TLR7a conjugate (an embodiment of a “rejuvenating” conjugate) to the subject having a folate receptor-expressing tumor, the “rejuvenating” conjugate bound to the same anti-fluorescein receptor on the surface of the CAR T-cell, thereby enabling endocytosis of the TLR7a by the CAR T-cell and rejuvenation of the CAR-T cell. The symbol legend in FIG. 2B applies to the graphical representations in both FIGS. 2A and 2B.

FIGS. 3A-3D support that fluorescein-conjugated fluorescent dyes only bound to anti-fluorescein CAR-expressing T-cells. FIG. 3A demonstrates that a fluorescein-near infrared (NIR) NIR dye conjugate (either fluorescein-NIR dye or fluorescein-Alexafluor 647) bound to anti-fluorescein CAR T-cells in a manner that can be quantitatively blocked by the addition of 1000-fold excess fluorescein. Flow cytometry was performed on anti-fluorescein CAR T-cells in the absence (blue histogram) or presence (green histogram) of the fluorescein-NIR dye conjugate (10 nM), or in the presence of both fluorescein-dye conjugate and 1000-fold excess fluorescein (magenta histogram). FIG. 3B demonstrates that CAR negative cell lines, MDA-MB-231 and KB cells, expressed no binding sites for fluorescein-dye conjugates (10 nM). FIG. 3C shows confocal microscopic evaluation of the internalization of fluorescein-Alexafluor 647 conjugate by CAR T-cells incubated first for 1 hour at 4° C. FIG. 3D shows confocal microscopic evaluation of the internalization of fluorescein-Alexafluor 647 conjugate by CAR T-cells after the cells from FIG. 3C were subsequently transferred to 37° C. for 4 hours. Bar=10 μm.

FIGS. 4A-4D show activation of human CD3+ T-cells upon administration of different concentrations of either a TLR7-54 or TLR7-1a agonist. FIG. 4A shows the percent increase in CD69+ cells after isolated human peripheral blood CD3+ T-cells were stimulated with anti-CD3+ mAb in the absence or presence of increasing concentrations of TLR-54 or TLR7-1a as measured by flow cytometry. FIG. 4B shows the percent increase in CD25+ cells after isolated human peripheral blood CD3+ T-cells were stimulated with anti-CD3+ monoclonal antibodies (mAb) in the absence or presence of increasing concentrations of TLR-54 or TLR7-1a as measured by flow cytometry. FIG. 4C shows the level of interferon gamma (INF-γ) in the 24-hour cell-free supernatants of the stimulated T-cells as measured by enzyme-linked immunosorbent assay (ELISA). FIG. 4D shows the level of tumor necrosis factor alpha (TNF-α) in the 24-hour cell-free supernatants of the stimulated T-cells as measured by ELISA. The data show an increase in these parameters over baseline levels (i.e., vehicle (DMSO-treated)). Bar graphs represent mean±standard deviation (SD), n=3.

FIGS. 5A-5E show the effect of CAR T-cell targeted and non-targeted TLR7-1a agonists on CAR T-cell exhaustion in vitro. FIG. 5A shows the protocol for induction of CAR T-cell exhaustion involving a serial transfer of CAR T-cells every 12 hours to fresh MDA-MB-231 human breast cancer cells in culture. FIG. 5B shows a decrease in the ability of the anti-fluorescein CAR T-cells to kill MDA-MB-231 cells after three rounds of serial transfer. FIG. 5C shows an increase in expression of T-cell exhaustion markers (programmed cell death protein 1 (PD-1⁺), T-cell immunoglobulin and mucin-domain containing-3 (TIM3⁺), and lymphocyte activating 3 (LAG3⁺)) after three rounds of serial transfer. FIG. 5D shows a return of the ability of the anti-fluorescein CAR T-cells to kill MDA-MB-231 cells in culture after incubation with targeted or non-targeted TLR7-1a. FIG. 5E shows a decrease in the expression of cell-surface exhaustion markers by rejuvenated anti-fluorescein CAR T-cells. The data show the change in these markers over baseline levels (i.e., vehicle (DMSO-treated)). Bar graphs present mean±SD, n=3.

FIGS. 6A-6E show the effect of anti-fluorescein CAR T-cell therapy on the growth and immunologic properties of MDA-MB-231 and KB tumors. NOD SCID gamma (NSG) mice were implanted with 4 million MDA-MB-231 cells and 1 million KB cells on separate flanks of the same mice and infused 2 weeks later with either saline or 8×10⁶ anti-fluorescein CAR T-cells (i.e., when MDA-MB-231 and KB tumors reached ˜160 or 80 mm³, respectively (i.e., to accommodate their different growth rates)). Four and 24 hours after CAR T-cell infusion, all mice were intravenously injected with 500 nmol/kg fluorescein-folate, and this injection was repeated once/week thereafter. FIG. 6A shows the tumor volume in cohorts either left untreated (dashed lines) or treated with both anti-fluorescein CAR T-cells plus fluorescein-folate bispecific adapter (solid lines). FIG. 6B shows the percentage of CD3+ T-cells (as a percentage of total cells in tumor) on day 18, when tumors were resected and both cancer and stromal cells were released using a tumor dissociation kit prior to analysis by flow cytometry. FIG. 6C shows the exhaustion markers PD-1+ and TIM3+ (as a percentage of all human CD3+ T-cells) on day 18, when tumors were resected and both cancer and stromal cells were released using a tumor dissociation kit prior to analysis by flow cytometry. FIG. 6D shows the ratio of CD86+ F4/80+ CD11b+ to CD206+ F4/80+ CD11b+ myeloid cells on day 18, when tumors were resected and both cancer and stromal cells were released using a tumor dissociation kit prior to analysis by flow cytometry. n=5 mice/group. All data were analyzed by two-way analysis of variance (ANOVA) and plotted as mean±SEM (*p<0.05, **p<0.01, ***p<0.001). Data shown are representative of two independent experiments. FIG. 6E shows representative images showing human CD3+ T-cell infiltrations into MDA-MB-231 and KB cell solid tumors. Bar=200 μm.

FIG. 7 shows that fluorescein-conjugated fluorescent dye (fluorescein-NIR dye) is specifically targeted to anti-fluorescein CAR T-cells in vivo. NSG mice were implanted with 1 million KB cells in one flank and infused with anti-fluorescein CAR T-cells (8×10⁶ cells that contained ˜50% anti-fluorescein CAR T-cells) when KB tumor volumes reached ˜50 mm³. 500 nmol/kg fluorescein-folate was injected at both 4 hours and 24 hours later and again once/week thereafter. Fifteen days after CAR T-cell infusion, mice were tail vein-injected with 500 nmol/kg fluorescein-NIR dye, and 4 hours later tumors were dissociated and analyzed by flow cytometry for uptake of fluorescein-NIR dye. CD3+ T-cells were detected with anti-human CD3 on the APC-Cy7 channel, and green fluorescent protein (GFP)-transfected anti-fluorescein CAR T-cells were detected using the GFP channel.

FIGS. 8A-8F show the rejuvenation of anti-fluorescein CAR T-cells in vivo following intravenous injection of fluorescein-TLR7-1a conjugate. FIG. 8A shows the scheme for in vivo studies. Mice were injected subcutaneously on day −7 with 10⁶ KB cells and then infused on day 1 with 8×10⁶ anti-fluorescein CAR T-cells. At 6 hours, 24 hours, and 9 days later, mice were intravenously injected with fluorescein-folate to induce engagement of CAR T-cells with folate receptor-positive KB cancer cells. Then on days 4-7 and days 11-14, mice were intravenously injected with fluorescein-TLR7-1a. Tumor volumes (FIG. 8B) and animal body weight changes (FIG. 8C) were measured every 3 days. Tumors were resected and dissociated into component cells on day 16 and human CD3⁺ T-cells were determined as a percentage of all tumor cells in FIG. 8D. PD-1⁺ TIM3⁺ cells as a percentage of all human CD3⁺ T-cells are shown in FIG. 8E, and the ratio of mouse CD86⁺ to CD206⁺ cells also expressing myeloid markers F4/80 and CD11b in the anti-fluorescein CAR T-cell treatment groups are shown in FIG. 8F. All data are plotted as mean±SEM. Data shown are representative of at least two independent experiments. Data were analyzed by two-way ANOVA, (**p<0.01; n.s., not significant).

While the present disclosure is susceptible to various modifications and alternative forms, exemplary embodiments thereof are shown by way of example in the drawings and are herein described in detail.

### DETAILED DESCRIPTION

The present disclosure is directed to the rejuvenation of exhausted chimeric antigen receptor (CAR) T-cells. Rejuvenation increases cell proliferation and reverses compromised effector function, thereby rendering the rejuvenated CAR T-cells useful for eradication of solid tumors.

CAR-T cells may become dysfunctional or “exhausted,” or reduced proliferation can result upon chronic exposure to tumor antigens or immunosuppressive factors (e.g., myeloid-derived suppressor cells (MDSCs), tumor-associated macrophages (TAMs), regulatory T cells (Tregs), and inhibitory cytokines) in the tumor microenvironment. To address this, in at least one embodiment, the endocytosis of a recognition region that is part of the CAR (e.g., a scFV fragment) is exploited to deliver a rejuvenating compound or conjugate, or a pharmaceutically acceptable salt thereof, to the CAR-T cells.

The rejuvenating conjugate, or a pharmaceutically acceptable salt thereof, comprises an active agent for rejuvenating CAR-T cells conjugated via a linker with a targeting moiety (e.g., that selectively binds a CAR of a CAR T-cell). The T-cell rejuvenating conjugate, or the pharmaceutically acceptable salt thereof, can be a compound, drug or active agent formulated to rejuvenate exhausted CAR-T cells. “Rejuvenating CAR-T cells” and its variants means activating CAR-T cells, increasing proliferation of CAR-T cells, blocking the inhibitory signaling of exhausted or dysfunctional CAR-T cells, re-activating CAR-T cells through an antigen-independent pathway, or otherwise increasing the function of CAR-T cells. Embodiments of the present conjugates can be particularly useful in preventing or reversing T cell exhaustion or dysfunction, reduced proliferation, and like conditions induced by the tumor microenvironment. In various embodiments, the rejuvenating compound, or the pharmaceutically acceptable salt thereof, is selected from a group consisting of a Toll-Like Receptor (TLR) agonist (e.g., a TLR7, TLR8, and/or TLR7 and TLR8 (an agonist of TLR7/8)).

As noted above, the T-cell rejuvenating conjugate further comprises a targeting moiety. The targeting moiety can selectively bind a CAR of a CAR T-cell. The terms “selectively binds,” “binds with specificity,” “binds with high affinity,” or “specifically binds,” when referring to a ligand/receptor, a recognition region/targeting moiety, or other binding pairs indicates a binding reaction that is determinative of the presence of the protein in a heterogeneous population of proteins and other biologics. Thus, under designated conditions, a specified ligand or targeting moiety binds to a particular receptor (e.g., one present on a cancer cell) or targeting moiety (e.g., one present on a CAR recognition region), respectively, and does not bind in a significant amount to other proteins present in the sample (e.g., those associated with normal, healthy cells). Specific binding or binding with specificity or high affinity can also mean, for example, that the binding moiety or ligand binds to its target with an affinity that is often at least 25% greater, more often at least 50% greater, most often at least 100% (2-fold) greater, normally at least ten-times greater, more normally at least 20-times greater, and most normally at least 100-times greater than the affinity with any other binding receptor.

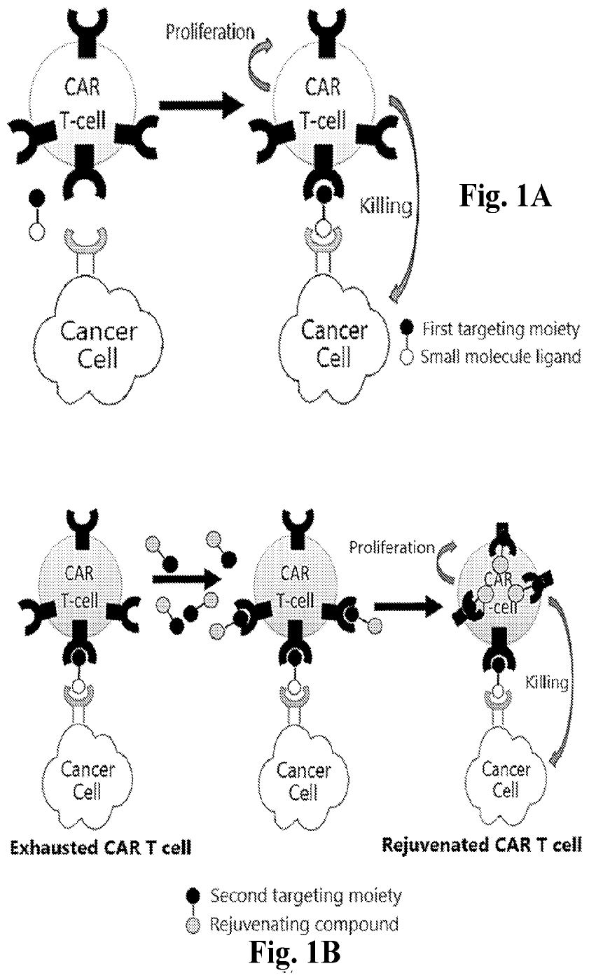

In certain embodiments, the conjugates hereof can be useful in applications when administered in conjunction with “cancer-killing” compounds. FIG. 1A shows the cell-killing effect of CAR T-cells in the presence of a “cancer-killing” conjugate (“cancer-killing” is used herein for ease of reference to distinguish from “rejuvenating”), which binds a CAR T-cell to a cancer cell. FIG. 1B shows the rejuvenation of CAR T-cells in the presence of the cancer-killing conjugate and a “rejuvenating” conjugate, which comprises a rejuvenating compound that binds to a receptor, e.g., a fluorescein receptor, on the CAR T-cell.

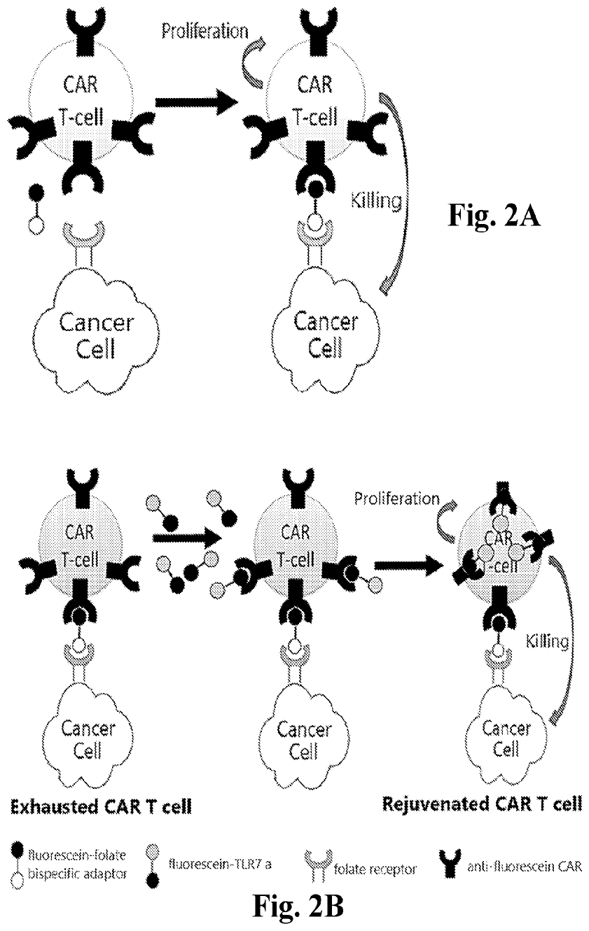

FIG. 2A shows that, upon administration of a fluorescein-folate conjugate (an embodiment of a “cancer-killing” conjugate) to a subject having a folate receptor-expressing tumor and anti-fluorescein CAR T-cells (i.e., CAR T-cells with an anti-fluorescein cell-surface receptor), the “cancer-killing” conjugate can form a bridge between an anti-fluorescein CAR T-cell and a folate receptor-expressing cancer cell, which promotes killing of the cancer cell and proliferation of the CAR T-cell. While a fluorescein receptor is referenced herein in various embodiments and examples, it will be appreciated that other receptors on the CAR T-cell can be targeted and/or bound by the rejuvenating conjugates hereof. FIG. 2B shows that, upon administration of a fluorescein-TLR7a conjugate (an embodiment of a “rejuvenating” conjugate) to a subject having a folate receptor-expressing tumor and exhausted anti-fluorescein CAR T-cells, the “rejuvenating” conjugate binds to the anti-fluorescein receptor on the surface of the CAR T-cell and the rejuvenating conjugate is endocytosed by the CAR T-cell, thereby leading to rejuvenation of the CAR-T cell.

In view of the above, provided is a method of rejuvenating exhausted CAR T-cells in a subject with a cancer being treated with a CAR T-cell therapy. The CAR T-cells can be cytotoxic lymphocytes such as cytotoxic T cells. In certain embodiments, the CAR can be used in connection with NK cells. In certain embodiments, the CAR can be used in connection with lymphokine-activated killer (LAK) cells. In certain embodiments, the CAR can be used in connection with a combination of NK cells, LAK cells, and/or T cells. It will be appreciated that various engineered cell therapies are now known in the art and, in certain embodiments, the CAR T-cell therapy can comprise any now known or hereinafter discovered engineered cells or cellular therapies that are useful for treating or preventing cancer and could benefit from use in conjunction with the CAR T-cell rejuvenating conjugates hereof.

In certain embodiments, the engineered cells are NK cells prepared from progenitor or stem cells. In certain embodiments, the engineered cells are T cells prepared from progenitor or stem cells.

In at least one embodiment, T lymphocytes (e.g., cytotoxic T lymphocytes) are engineered to express CAR. In at least one embodiment, the NK cells are engineered to express CAR.

The CAR is a fusion protein comprising a recognition region, a co-stimulation domain, and an activation signaling domain. In certain embodiments, the CAR binds a cell-surface antigen on an immunosuppressive cell or a cancerous cell with high specificity.

In certain embodiments, the recognition region of the CAR can be a scFv of an antibody, a Fab fragment or the like that binds to a cell-surface antigen (e.g., cluster of differentiation 19 (CD19)) with specificity (e.g., high specificity). Where the recognition region of the CAR comprises a scFv region, the scFv region can be prepared from (i) an antibody known in the art that binds a targeting moiety, (ii) an antibody newly prepared using at least one targeting moiety such as a hapten, and (iii) sequence variants derived from the scFv regions of such antibodies, e.g., scFv regions having at least about 80%, at least about 90%, at least about 91%, at least about 92%, at least about 93%, at least about 94%, at least about 95%, at least about 96%, at least about 97%, at least about 98%, at least about 99%, or at least about 99.5% sequence identity with the amino acid sequence of the scFv region from which they are derived.

“Percent (%) sequence identity” with respect to a reference to a polypeptide sequence or a nucleotide sequence is defined as the percentage of amino acid or nucleic acid residues, respectively, in a candidate sequence that are identical with the residues in the reference sequence, after aligning the sequences and introducing gaps, if necessary, to achieve the maximum percent sequence identity and not considering any conservative substitutions as part of the sequence identity. Alignment for purposes of determining percent sequence identity can be achieved in various ways that are within the skill of the art, for instance, using publicly available computer software. For example, determination of percent identity or similarity between sequences can be done, for example, by using the GAP program (Genetics Computer Group, software; now available via Accelrys online), and alignments can be done using, for example, the ClustalW algorithm (VNTI software, InforMax Inc., Bethesda, MD). Further, a sequence database can be searched using the nucleic acid or amino acid sequence of interest. Algorithms for database searching are typically based on the BLAST software, but those skilled in the art can determine appropriate parameters for aligning sequences, including any algorithms needed to achieve maximal alignment over the full length of the sequences being compared. In some embodiments, the percent identity can be determined along the full length of the nucleic acid or amino acid sequence.

In certain embodiments, the activation signaling domain generates a lymphocyte activation signal upon binding of the CAR to a targeting moiety. Suitable activation signaling domains can be, without limitation, a T cell CD3 chain, a CD3 delta receptor protein, mbl receptor protein, B29 receptor protein, a Fc receptor γ, 4-1BB domain, a CD28 activation domain, or an IL-15/IL-2 domain. The skilled artisan will understand that sequence variants of these activation signaling domains can be used where the variants have the same or similar activity as the domain upon which they are modeled. In various embodiments, the variants have at least about 80%, at least about 90%, at least about 91%, at least about 92%, at least about 93%, at least about 94%, at least about 95%, at least about 96%, at least about 97%, at least about 98%, at least about 99%, or at least about 99.5% sequence identity with the amino acid sequence of the domain from which they are derived.

Constructs encoding the CARs can be prepared using genetic engineering techniques. Such techniques are described in detail in Sambrook et al., “Molecular Cloning: A Laboratory Manual,” 3^{rd} Edition, Cold Spring Harbor Laboratory Press, (2001), and Green and Sambrook, “Molecular Cloning: A Laboratory Manual,” 4^{th} Edition, Cold Spring Harbor Laboratory Press, (2012), which are both incorporated herein by reference in their entireties (collectively, the “Protocols”).

By way of non-limiting examples, a plasmid or viral expression vector (e.g., a lentiviral vector, a retrovirus vector, sleeping beauty, and piggyback (transposon/transposase systems that include a non-viral mediated CAR gene delivery system)) can be prepared that encodes a fusion protein comprising a recognition region, one or more co-stimulation domains, and an activation signaling domain, in frame and linked in a 5′ to 3′ direction.

Other arrangements are also acceptable and include a recognition region, an activation signaling domain, and one or more co-stimulation domains.

The term “vector” means any nucleic acid that functions to carry, harbor, or express a nucleic acid of interest. Nucleic acid vectors can have specialized functions such as expression, packaging, pseudotyping, or transduction. Vectors can also have manipulatory functions if adapted for use as a cloning or shuttle vector. The structure of the vector can include any desired form that is feasible to make and desirable for a particular use. Such forms can include, for example, circular forms such as plasmids and phagemids, as well as linear or branched forms. A nucleic acid vector can be composed of, or example, DNA or RNA, as well as contain partially or fully, nucleotide derivatives, analogs or mimetics. Such vectors can be obtained from natural sources, produced recombinantly or chemically synthesized.

The placement of the recognition region in the fusion protein will generally be such that display of the region on the exterior of the cell is achieved. Where desired, the CARs can also include additional elements, such as a signal peptide (e.g., CD8α signal peptide) to ensure proper export of the fusion protein to the cell surface, a transmembrane domain to ensure the fusion protein is maintained as an integral membrane protein (e.g., CD8α transmembrane domain, CD28 transmembrane domain, or CD3ζ transmembrane domain), and a hinge domain (e.g., CD8α hinge) that imparts flexibility to the recognition region and allows strong binding to the targeting moiety.

Cytotoxic lymphocytes (e.g., cytotoxic T lymphocytes or NK cells) can be genetically engineered to express CAR constructs by transfecting a population of the lymphocytes with an expression vector encoding the CAR construct. Suitable methods for preparing a transduced population of lymphocytes expressing a selected CAR construct are well-known to the skilled artisan.

In one embodiment, the cells used in the methods described herein can be autologous cells, although heterologous cells can also be used, such as when the patient being treated has received high-dose chemotherapy or radiation treatment to destroy the patient's immune system. In one embodiment, allogenic cells can be used.

In connection with the methods hereof, the CAR T-cell therapy can comprise a therapy where the subject receives T-cells expressing a CAR. In certain embodiments, the CAR T-cell therapy can be administered to the subject via a vector that comprises a promoter operably linked to a nucleic acid sequence encoding a CAR. The CAR can bind a first targeting moiety, a second targeting moiety, or both the first targeting moiety and the second targeting moiety. In certain embodiments, the CAR can bind the first targeting moiety, the second targeting moiety, or both the first and second targeting moieties with specificity.

The CAR T-cell therapy further comprises the subject receiving a cancer-binding conjugate comprising a ligand conjugated with at least the first targeting moiety of the CAR. The ligand can bind a receptor on a cancer cell (e.g., the ligand can bind the cancer cell receptor with specificity). In certain embodiments, the ligand and the first targeting moiety are conjugated via a first linker. As used herein, the term “ligand” is a molecule, ion, or atom that is attached to the central atom or ion (e.g., a drug) of a conjugate.

The method further comprises administering to the subject (e.g., with exhausted CAR T-cells) a CAR T-cell-rejuvenating conjugate. The CAR T-cell-rejuvenating conjugate can comprise an agonist of toll-like receptor 7 (TLR7), toll-like receptor 8 (TLR8), or toll-like receptor 7 and toll-like receptor 8 (TLR7/8) conjugated with either the first targeting moiety or the second targeting moiety (such that, for example, the CAR can bind the first targeting moiety and/or the second targeting moiety). In certain embodiments, the agonist and either of the first targeting moiety or the second targeting moiety are optionally conjugated via a second linker.

The CAR T-cell-rejuvenating conjugate has the structure:

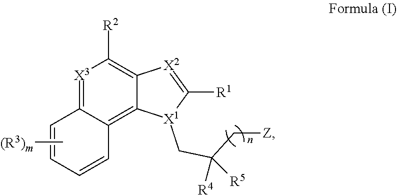

or is a pharmaceutically acceptable salt thereof, wherein:
   R¹ is an acyclic or cyclic alkyl, which is optionally substituted with one or more substituents independently selected from an alkyl, halo, heteroalkyl, alkoxy, and cycloalkyl;
   R² is —NR^{2x}R^{2y}, H, —OR^{z}, —SO₂N(R^{z})₂, or N₃, wherein:
      R^{2x} and R^{2y} are each independently hydrogen, —N(R^{z})₂, —CON(R^{z})₂, —C(R^{z})₂—N(R^{z})₂, —CS—N(R^{z})₂, or an alkyl, which is optionally substituted with one or more substituents independently selected from oxo, halo, alkyl, heteroalkyl, alkoxy, and cycloalkyl;
      each R^{z} is independently hydrogen or alkyl that is optionally substituted; or
      R^{2x} and R^{2y} are taken together to form a 3-10 membered mono- or bicyclic heterocycloalkyl, which is optionally substituted;
   each R³ is independently hydrogen, halo, —N₃, —CN, —NO₂, alkyl, heteroalkyl, cycloalkyl, heterocycloalkyl, alkoxy, aryl, heteroaryl, heterocycloalkyl, amino, hydroxy, carbonyl, or thiol, wherein the alkyl, heteroalkyl, cycloalkyl, heterocycloalkyl, or alkoxy that is optionally substituted;
   R⁴ and R⁵ are each independently alkyl, alkoxy, halo, or cycloalkyl, wherein the alkyl, alkoxy, or cycloalkyl is optionally substituted;
   X¹, X², and X³ are independently N or CR^{q}, wherein each R^{q} is independently hydrogen (H), halo, or optionally substituted alkyl;
   Z is G-L-, wherein L is a linker and G is the first targeting moiety or the second targeting moiety (e.g., that the CAR binds with specificity);
   n in Formula (I) is 1-6; and
   m in Formula (I) is 0-4.

In certain embodiments, the CAR T-cell-rejuvenating conjugate has the structure:

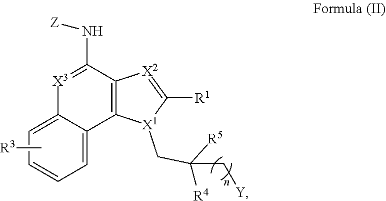

or is a pharmaceutically acceptable salt thereof, wherein:
   R¹, R³, R⁴, and R⁵ are each independently H, alkyl, alkoxyl, alkenyl, alkynyl, alicyclic, aryl, biaryl, halo, heteroaryl, —COR^{2x}

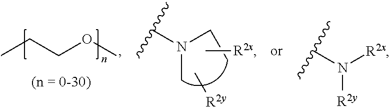

wherein each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl;
   Z is G-L-, wherein L is a linker and G is the first targeting moiety or the second targeting moiety (e.g., that can bind the CAR with specificity);
   X¹, X², and X³ are independently CR^{q} or N, wherein each R^{q} is independently H, halo, or optionally substituted alkyl;
   Y is H, —OR^{z}, —NR^{2x}R^{2y}, —SR^{z}, —SOR^{z}, —SO₃R^{z}, —N₃, —COR^{z}, —COOR^{z}, —CON(R^{z})₂, —COSR^{z}, —SO₂N(R^{z})₂, or —CON(R^{z})₂, wherein:
      R^{2x} and R^{2y} are each independently H, —N(R^{z})₂, —CON(R^{z})₂, —C(R^{z})₂—N(R^{z})₂, —CS—N(R^{z})₂, or optionally substituted alkyl (e.g., optionally substituted with one or more substituent, each substituent independently being oxo, halogen, alkyl, heteroalkyl, alkoxy, or cycloalkyl);
      each R^{z} is independently H, or optionally substituted alkyl; or
      R^{2x} and R^{2y} are taken together to form an optionally substituted heterocycloalkyl (e.g., wherein the optionally substituted heterocycloalkyl is a mono- or bicyclic heterocycloalkyl and/or wherein the optionally substituted heterocycloalkyl is a 3-10 membered heterocycloalkyl); and
   n in Formula (11) is 0-30.

In certain embodiments, the CAR T-cell-rejuvenating conjugate has the structure:

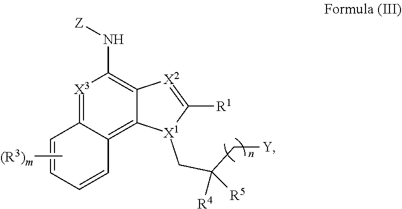

or is a pharmaceutically acceptable salt thereof, wherein:
   R¹ is an acyclic or cyclic alkyl, which is optionally substituted with one or more substituents independently selected from halo, alkyl, heteroalkyl, alkoxy, and cycloalkyl;
   Y is H, —OR^{z}, —NR^{2x}R^{2y}, —SR^{z}, —SOR^{z}, —SO₃R^{z}, —N₃, —COR^{z}, —COOR^{z}, —CONR^{z}₂, —COSR^{z}, —SO₂N(R^{z})₂, or —CON(R^{z})₂, wherein:
      R^{2x} and R^{2y} are each independently H, —N(R^{z})₂, —CON(R^{z})₂, —C(R^{Z})₂—N(R^{z})₂, —CS—N(R^{z})₂, or alkyl, which is optionally substituted with one or more substituents independently selected from oxo, halo, alkyl, heteroalkyl, alkoxy, and cycloalkyl;
   each R^{z} is independently hydrogen, or optionally substituted alkyl; or
      R^{2x} and R^{2y} are taken together to form a 3-10 membered mono- or bicyclic heterocycloalkyl, which is optionally substituted;
   each R³ is independently halo, —N₃, —CN, —NO₂, alkyl, heteroalkyl, cycloalkyl, heterocycloalkyl, alkoxy, aryl, heteroaryl, heterocycloalkyl, amino, hydroxy, carbonyl, or thiol, wherein the alkyl, alkoxy, heteroalkyl, cycloalkyl, or heterocycloalkyl is optionally substituted;
   R⁴ and R⁵ are each independently alkyl, alkoxy, halogen, or cycloalkyl, wherein the alkyl, alkoxy, or cycloalkyl is optionally substituted;
   X¹, X², and X³ are independently CR^{q} or N, wherein each R^{q} is independently H, halogen, or optionally substituted alkyl;
   Z is L-G, wherein L is a linker and G is the first targeting moiety or the second targeting moiety;
   n in Formula (III) is 0-30; and
   m in Formula (III) is 0-4.

In certain embodiments, when the CAR T-cells in the subject (e.g., exhausted CAR T-cells) bind and endocytose the CAR T-cell-rejuvenating conjugate, the CAR T-cells are rejuvenated by the agonist thereof. In various embodiments, each of X¹, X², and X³ of Formula (I), (II), and/or (III) can be nitrogen (N).

The CAR T-cell-rejuvenating conjugate can have the structure:

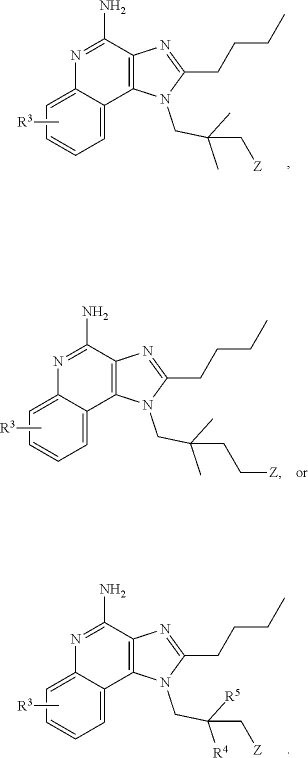

or be a pharmaceutically acceptable salt thereof. Alternatively, the CAR T-cell-rejuvenating conjugate can have the structure:

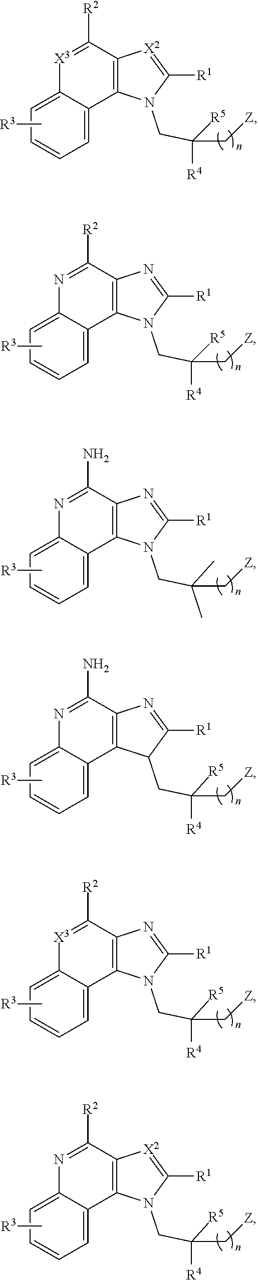

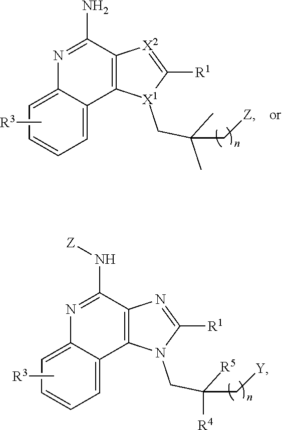

or can be a pharmaceutically acceptable salt of any of the foregoing structures. Also, alternatively, the CAR T-cell-rejuvenating agonist can have the structure:

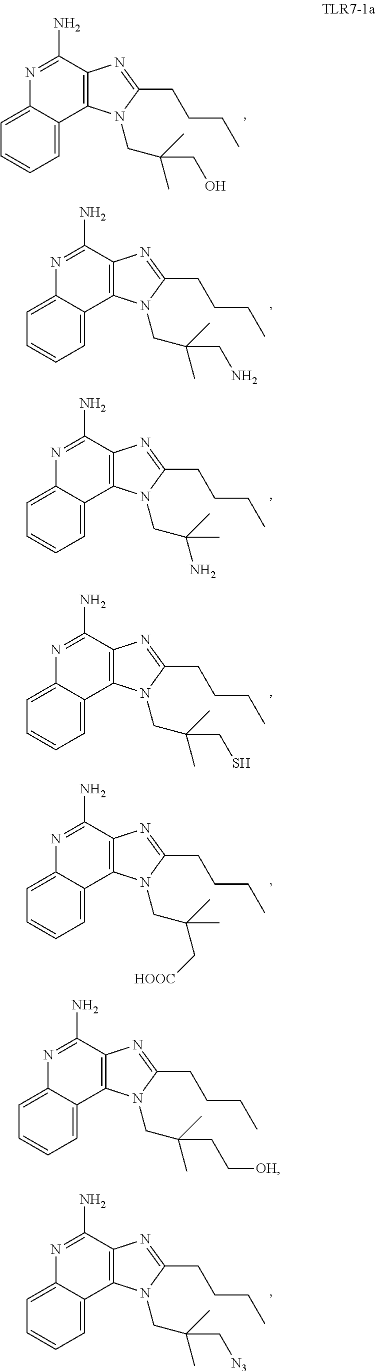

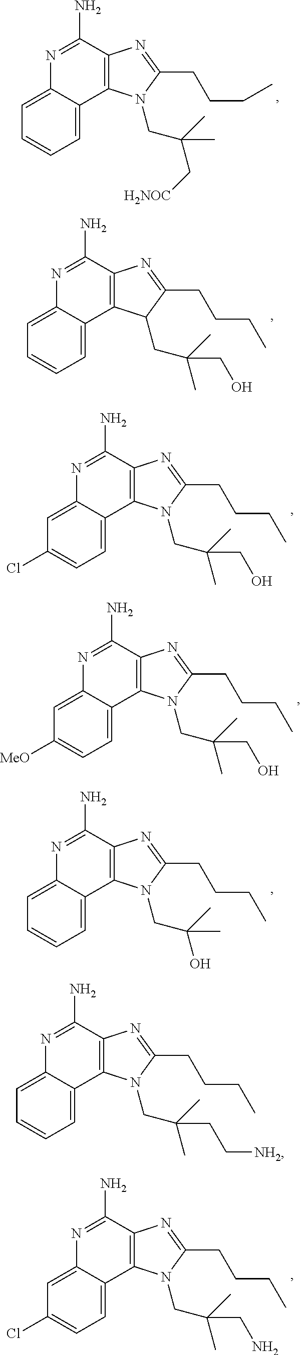

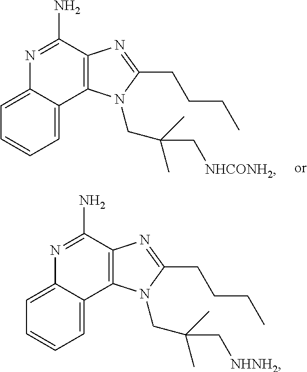

or can be a pharmaceutically acceptable salt thereof.

The first targeting moiety, the second targeting moiety, or the first targeting moiety and the second targeting moiety can be a group or can comprise a group with the structure:

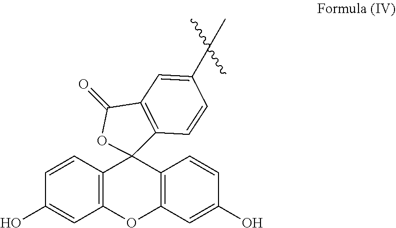 [SMILES: *c1ccc2c(c1)C(=O)OC21c2ccc(O)cc2Oc2cc(O)ccc21]

or a pharmaceutically acceptable salt thereof.

Alternatively, the first targeting moiety, the second targeting moiety, or the first targeting moiety and the second targeting moiety can be a group or can comprise a group with the structure:

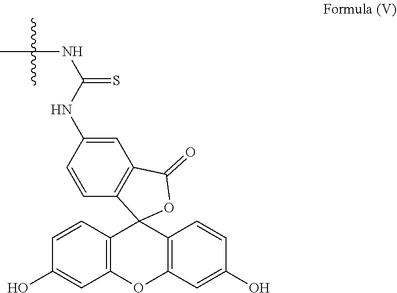 [SMILES: *NC(=S)Nc1ccc2c(c1)C(=O)OC21c2ccc(O)cc2Oc2cc(O)ccc21]

of a pharmaceutically acceptable salt thereof.

The CAR T-cell-rejuvenating conjugate can have the structure:

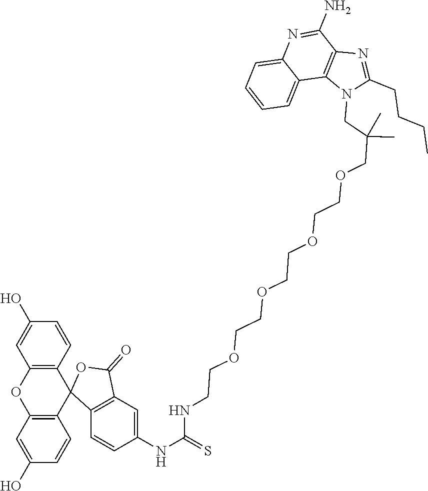 [SMILES: CCCCc1nc2c(N)nc3ccccc3c2n1CC(C)(C)COCCOCCOCCOCCNC(=S)Nc1ccc2c(c1)C(=O)OC21c2ccc(O)cc2Oc2cc(O)ccc21]

or a pharmaceutically acceptable salt thereof. Alternatively, the CAR T-cell-rejuvenating conjugate can have a structure selected from:

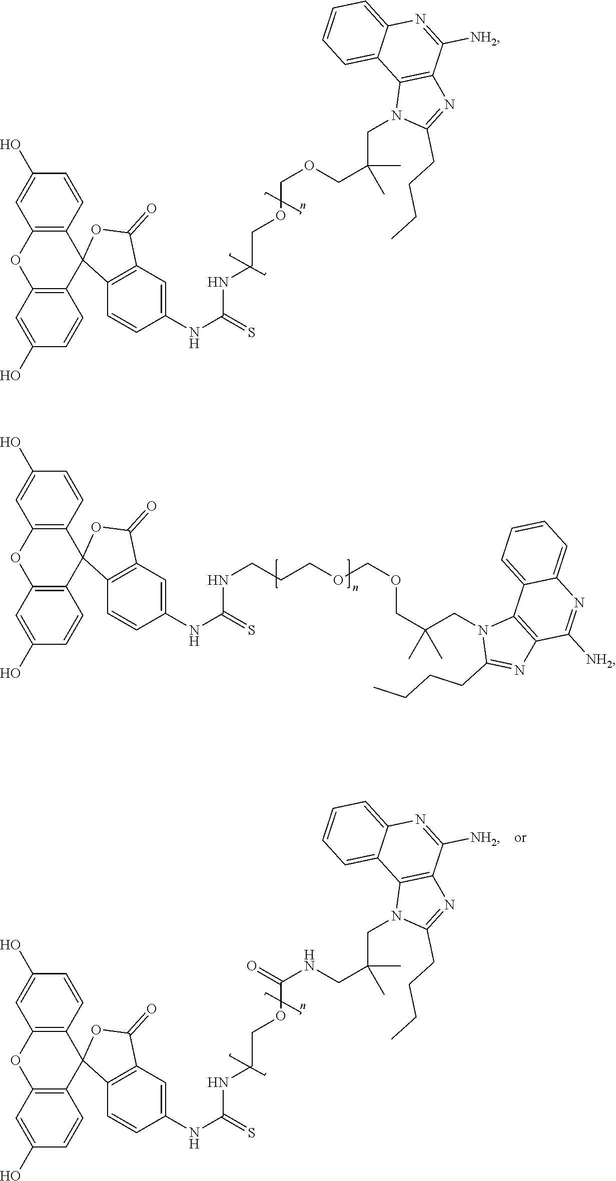

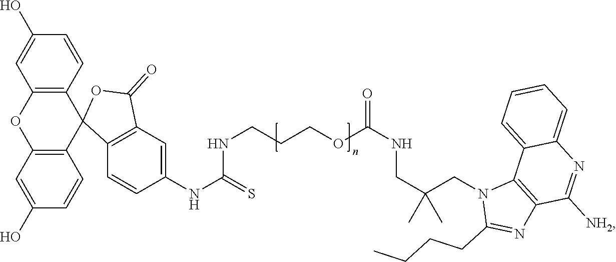

wherein n=0-200 or can be a pharmaceutically acceptable salt of any of the foregoing structures. Further alternatively, the CAR T-cell-rejuvenating conjugate can have a structure selected from:

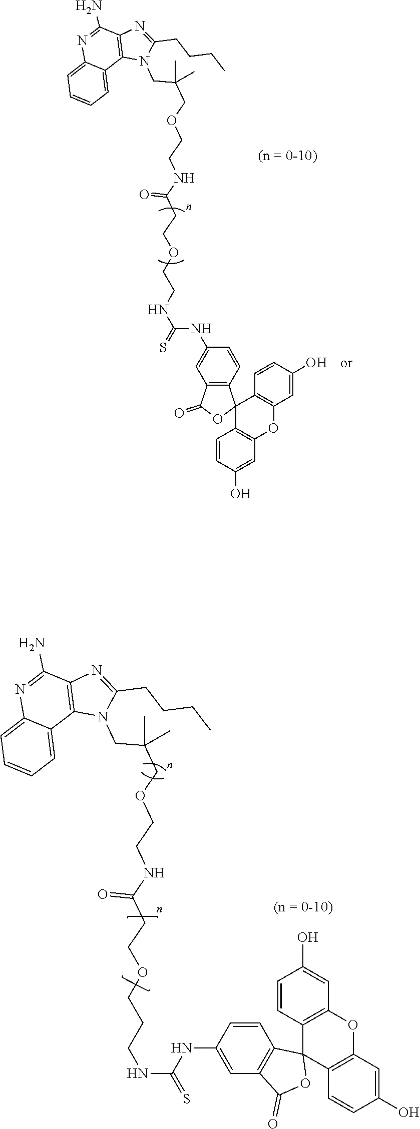

or can be pharmaceutically acceptable salt thereof. Even still further alternatively, the CAR T-cell-rejuvenating conjugate can have a structure selected from:

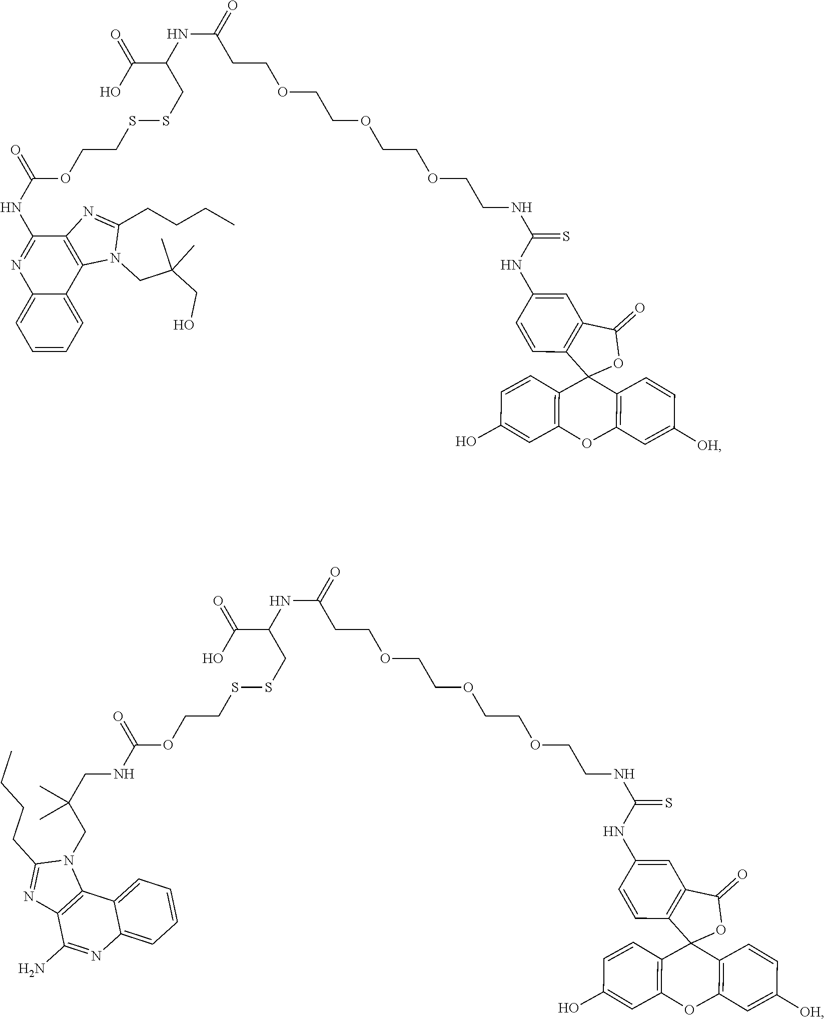 [SMILES: CCCCc1nc2c(NC(=O)OCCSSCC(NC(=O)CCOCCOCCOCCNC(=S)Nc3ccc4c(c3)C(=O)OC43c4ccc(O)cc4Oc4cc(O)ccc43)C(=O)O)nc3ccccc3c2n1CC(C)(C)CO ; CCCCc1nc2c(N)nc3ccccc3c2n1CC(C)(C)CNC(=O)OCCSSCC(NC(=O)CCOCCOCCOCCNC(=S)Nc1ccc2c(c1)C(=O)OC21c2ccc(O)cc2Oc2cc(O)ccc21)C(=O)O]

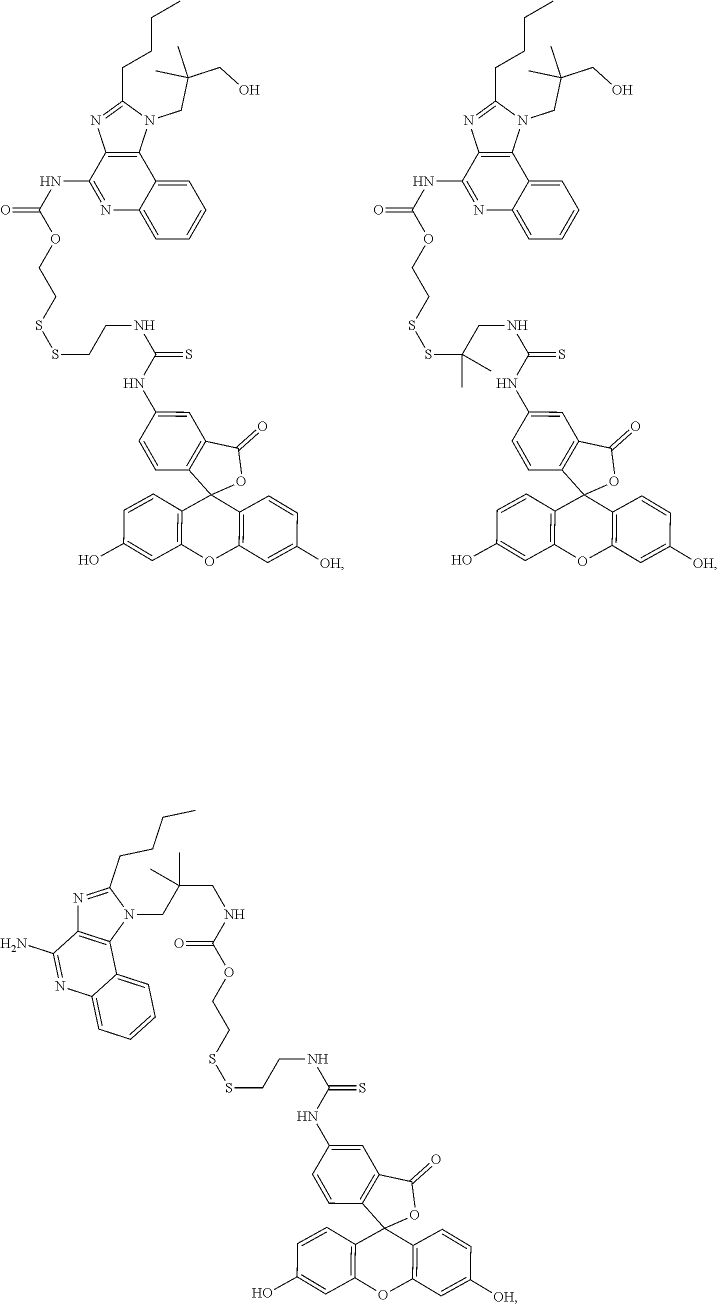 [SMILES: CCCCc1nc2c(NC(=O)OCCSSCCNC(=S)Nc3ccc4c(c3)C(=O)OC43c4ccc(O)cc4Oc4cc(O)ccc43)nc3ccccc3c2n1CC(C)(C)CO ; CCCCc1nc2c(NC(=O)OCCSSC(C)(C)CNC(=S)Nc3ccc4c(c3)C(=O)OC43c4ccc(O)cc4Oc4cc(O)ccc43)nc3ccccc3c2n1CC(C)(C)CO ; CCCCc1nc2c(N)nc3ccccc3c2n1CC(C)(C)CNC(=O)OCCSSCCNC(=S)Nc1ccc2c(c1)C(=O)OC21c2ccc(O)cc2Oc2cc(O)ccc21]

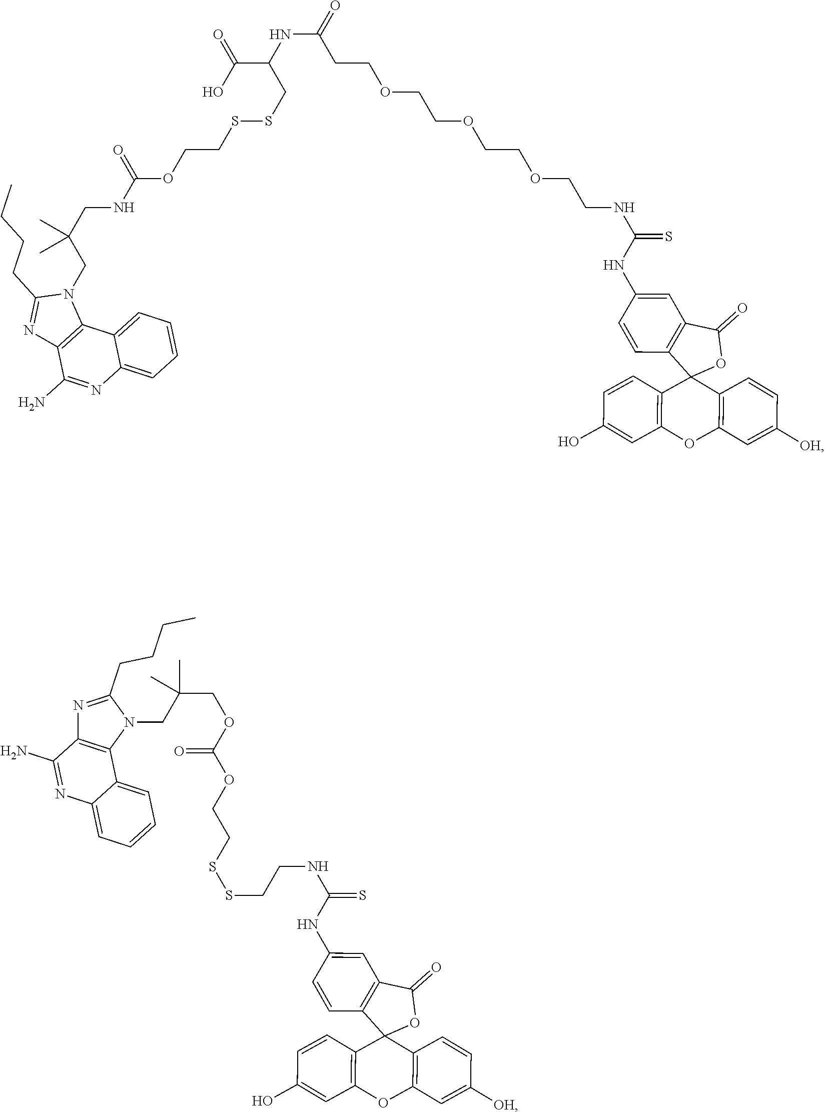 [SMILES: CCCCc1nc2c(N)nc3ccccc3c2n1CC(C)(C)CNC(=O)OCCSSCC(NC(=O)CCOCCOCCOCCNC(=S)Nc1ccc2c(c1)C(=O)OC21c2ccc(O)cc2Oc2cc(O)ccc21)C(=O)O ; CCCCc1nc2c(N)nc3ccccc3c2n1CC(C)(C)COC(=O)OCCSSCCNC(=S)Nc1ccc2c(c1)C(=O)OC21c2ccc(O)cc2Oc2cc(O)ccc21]

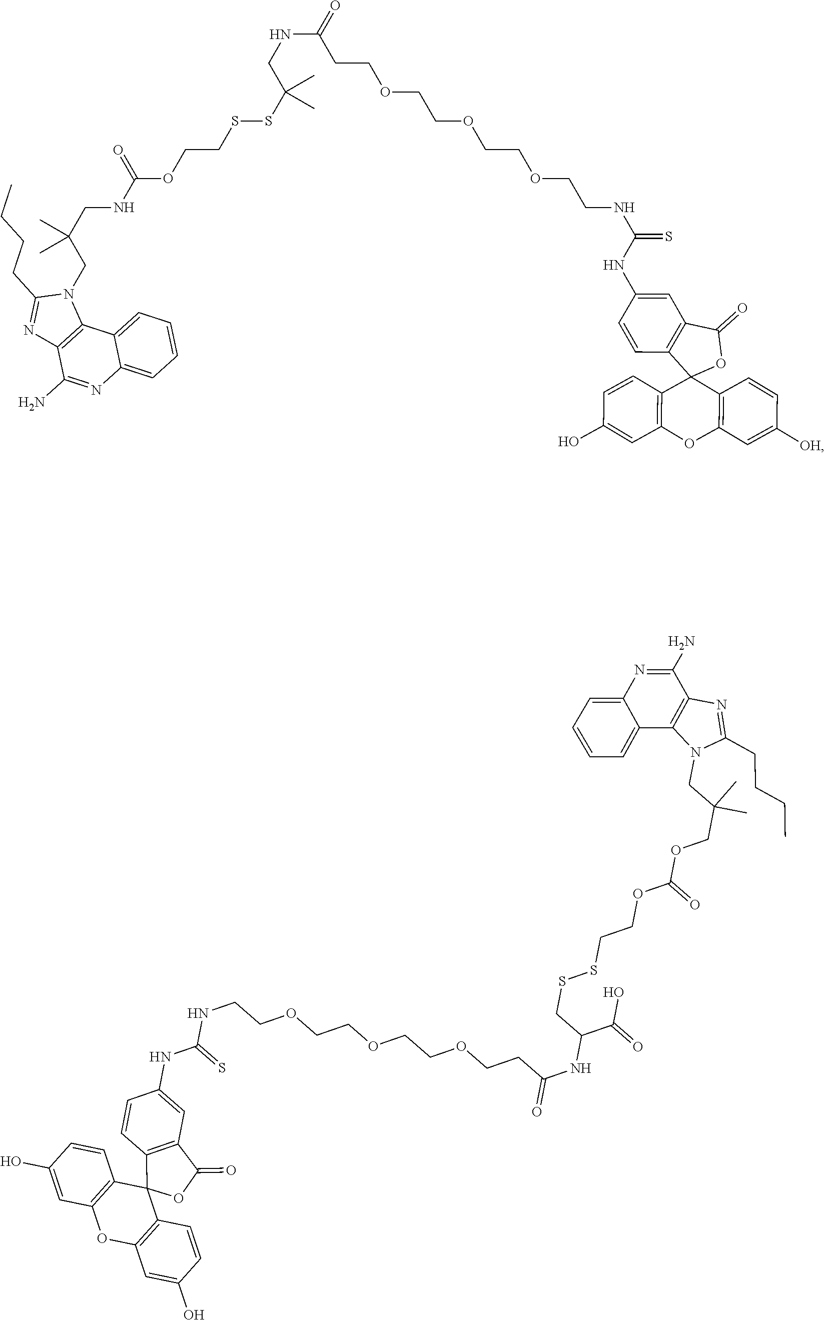 [SMILES: CCCCc1nc2c(N)nc3ccccc3c2n1CC(C)(C)CNC(=O)OCCSSC(C)(C)CNC(=O)CCOCCOCCOCCNC(=S)Nc1ccc2c(c1)C(=O)OC21c2ccc(O)cc2Oc2cc(O)ccc21 ; CCCCc1nc2c(N)nc3ccccc3c2n1CC(C)(C)COC(=O)OCCSSCC(NC(=O)CCOCCOCCOCCNC(=S)Nc1ccc2c(c1)C(=O)OC21c2ccc(O)cc2Oc2cc(O)ccc21)C(=O)O]

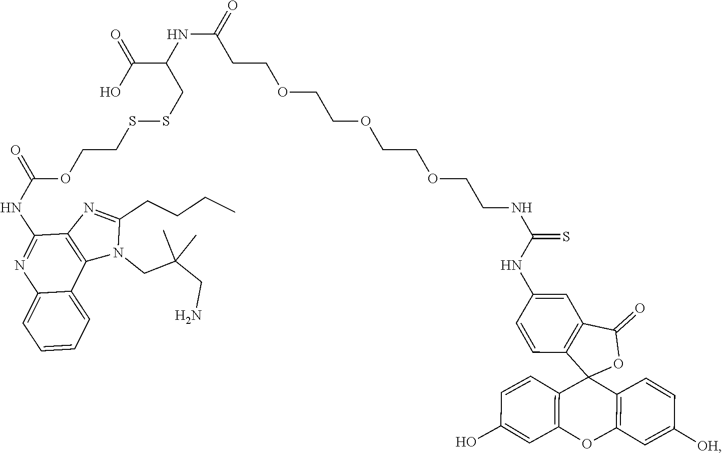 [SMILES: CCCCc1nc2c(NC(=O)OCCSSCC(NC(=O)CCOCCOCCOCCNC(=S)Nc3ccc4c(c3)C(=O)OC43c4ccc(O)cc4Oc4cc(O)ccc43)C(=O)O)nc3ccccc3c2n1CC(C)(C)CN]

or can be a pharmaceutically acceptable salt of any of the foregoing structures. Other structures for the CAR T-cell-rejuvenating conjugate include:

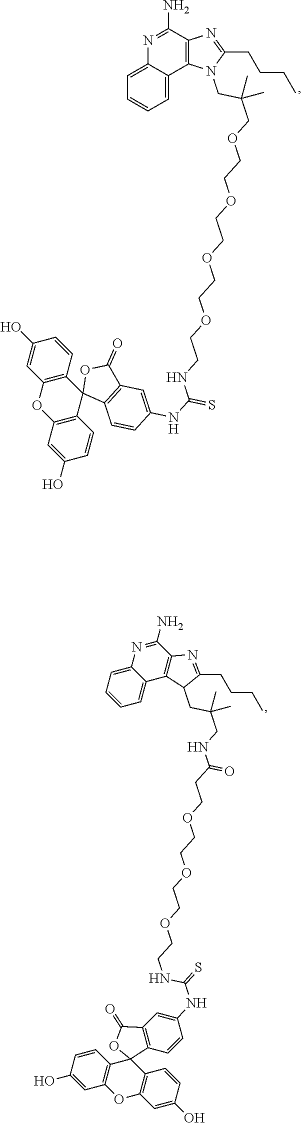 [SMILES: CCCCc1nc2c(N)nc3ccccc3c2n1CC(C)(C)COCCOCCOCCOCCNC(=S)Nc1ccc2c(c1)C(=O)OC21c2ccc(O)cc2Oc2cc(O)ccc21 ; CCCCC1=Nc2c(N)nc3ccccc3c2C1CC(C)(C)CNC(=O)CCOCCOCCOCCNC(=S)Nc1ccc2c(c1)C(=O)OC21c2ccc(O)cc2Oc2cc(O)ccc21]

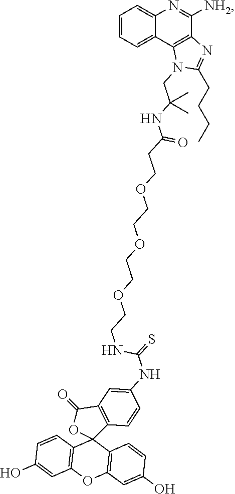 [SMILES: CCCCc1nc2c(N)nc3ccccc3c2n1CC(C)(C)NC(=O)CCOCCOCCOCCNC(=S)Nc1ccc2c(c1)C(=O)OC21c2ccc(O)cc2Oc2cc(O)ccc21]

or a pharmaceutically acceptable salt thereof. Still other structures for the CAR T-cell-rejuvenating conjugate include:

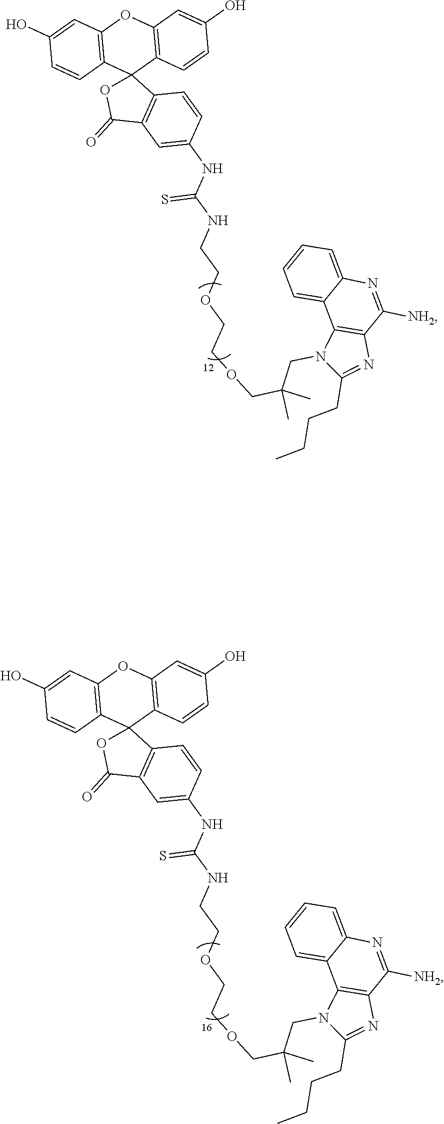

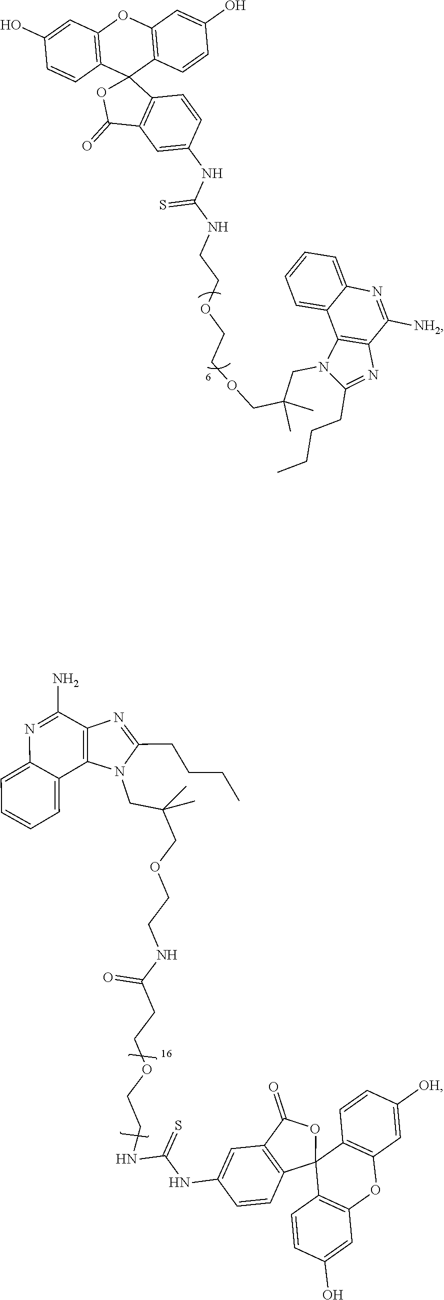

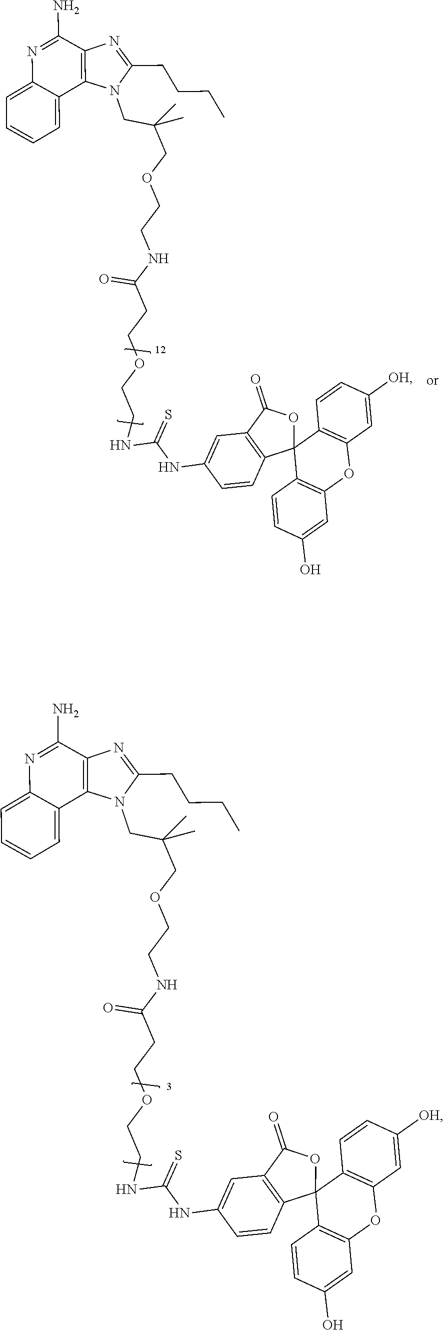

or a pharmaceutically acceptable salt of any of the foregoing structures.

In certain embodiments, the CAR T-cell-rejuvenating conjugate can have a structure selected from:

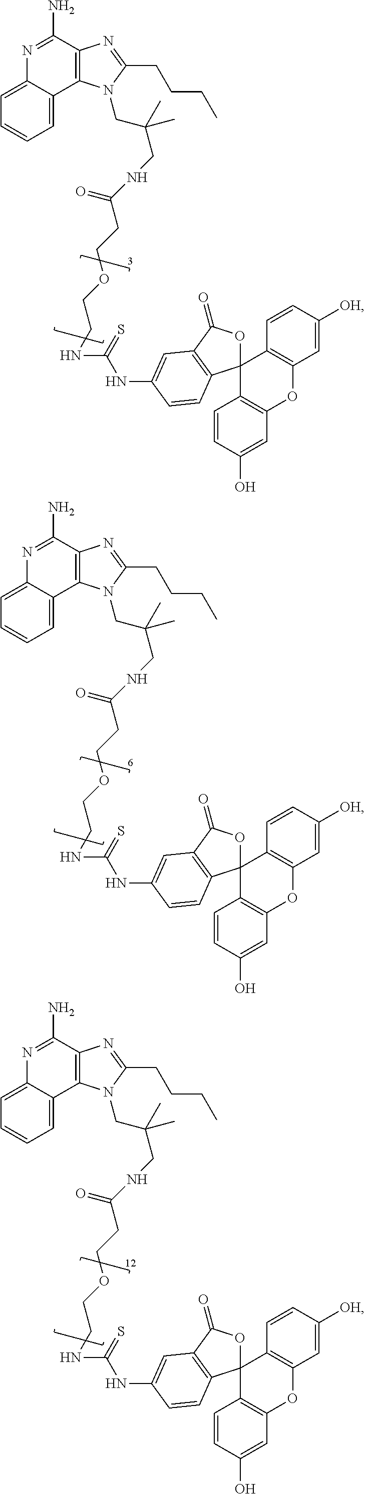

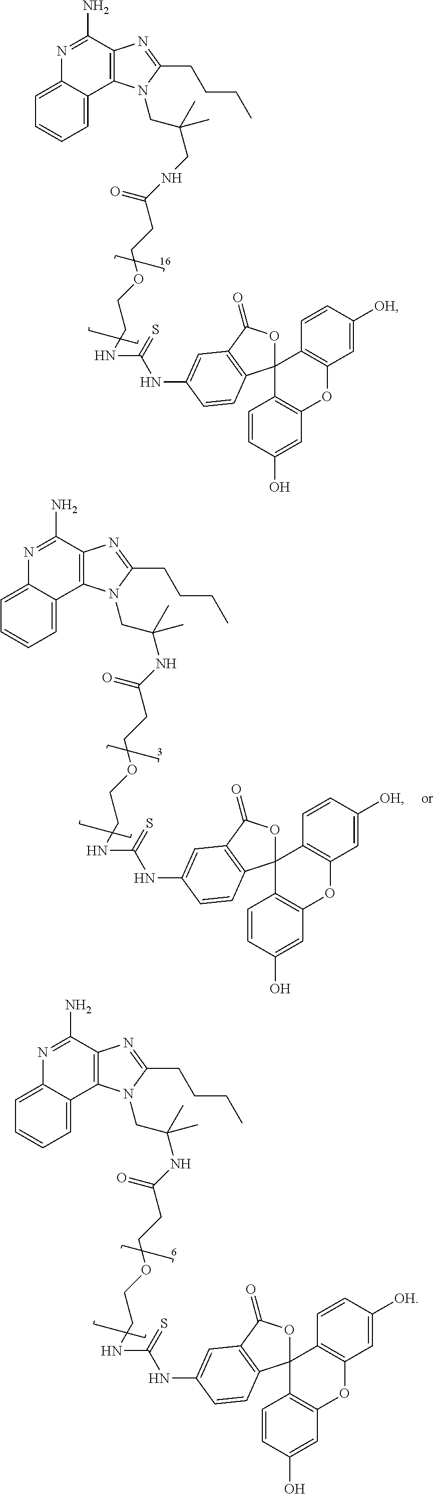

or is a pharmaceutically acceptable salt of any of the foregoing structures.

In some embodiments, as described above, the CAR T-cell therapy comprises a cancer-binding conjugate comprising a ligand and the first targeting moiety, wherein the ligand and first targeting moiety are optionally conjugated via the first linker.

The ligand can be bound by (or have affinity for binding or bind with specificity) a cancer (e.g., a cancer cell). The ligand bound by (or with an affinity to bind or that binds with specificity) the cancer cell can be selected from the group consisting of a folate, 5-methyltetrahydrofolate, a 2-[3-(1,3-dicarboxypropyl)ureido]pentanedioic acid (DUPA) ligand, a neurokinin 1 receptor (NK-1R) ligand, a carbonic anhydrase IX (CAIX) ligand, a ligand of gamma glutamyl transpeptidase, a ligand of luteinizing hormone releasing hormone (LHRHR), a ligand of CD73, a ligand of fibroblast activation protein, a ligand of heat shock protein (HSP), a ligand of glucose transporter 1 (glut-1), a ligand of a natural killer group 2D receptor (NKG2D) ligand, and a cholecystokinin B receptor (CCKBR or CCK2) ligand.

“Folate” can be folic acid, a folic acid analog, or another folate receptor-binding molecule, including for example, analogs and derivatives of folic acid such as, without limitation, folinic acid (e.g., leucovorin), pteroylpolyglutamic acid, pteroyl-D-glutamic acid, and folate receptor-binding pterdines such as tetrahydropterins, dihydrofolates, tetrahydrofolates (e.g., 5-methyltetrahydrofolate (5-MTHF)), and their deaza and dideaza analogs.

An “analog” or “derivative” with reference to a peptide, polypeptide or protein refers to another peptide, polypeptide or protein that possesses a similar or identical function as the original peptide, polypeptide or protein, but does not necessarily comprise a similar or identical amino acid sequence or structure of the original peptide, polypeptide or protein. An analog preferably satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the original amino acid sequence; (b) a proteinaceous agent encoded by a nucleotide sequence that hybridizes under stringent conditions to a nucleotide sequence encoding the original amino acid sequence; or (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding the original amino acid sequence.

The terms “deaza” and “dideaza” analogs refer to the art-recognized analogs having a carbon atom substituted for one or two nitrogen atoms in the naturally occurring folic acid structure, or analog or derivative thereof. For example, the deaza analogs can include the 1-deaza, 3-deaza, 5-deaza, 8-deaza, and 10-deaza analogs of folate, folinic acid, pteropolyglutamic acid, and folate receptor-binding pteridines such as tetrahydropterins, dihydrofolates, and tetrahydrofolates. The dideaza analogs include, for example, 1,5-dideaza, 5,10-dideaza, 8,10-dideaza, and 5,8-dideaza analogs. The foregoing folic acid analogs are conventionally termed “folates,” reflecting their capacity to bind to folate receptors. Other folate receptor-binding analogs include aminopterin, amethopterin (methotrexate), N¹⁰-methylfolate, 2-deamino-hydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate).

The foregoing analogs and/or derivatives are also termed “a folate,” “the folate,” or “folates” reflecting their ability to bind to folate-receptors. Such molecules, when conjugated with exogenous molecules, can be effective to enhance transmembrane transport, such as via folate-mediated endocytosis. The foregoing can be used in the folate receptor-binding ligands described herein.

The first targeting moiety and the second targeting moiety (e.g., with the affinity to bind the CAR) can be independently selected from the group consisting of 2,4-dinitrophenyl (DNP), L-rhamnose, tacrolimus (FK506), 2,4,6-trinitrophenol (TNP), biotin, rapamycin, digoxigenin, folate, 5-methyl tetrahydrofolate, fluorescein, fluorescein isothiocyanate (FITC), NHS-fluorescein, , pentafluorophenyl ester, tetrafluorophenyl ester, knottin, centyrin, DARPin, an affibody, an affilin, an anticalin, an atrimer, an avimer, a bicicyclic peptide, an FN3 scaffold, a cys-knot, a fynomer, a Kunitz domain, or an Obody.

The first linker and the second linker can be independently releasable or non-releasable, where applicable. The first linker and the second linker can independently comprise C₁-C₂₀ alkyl, alkylene, heteroalkylene, —O-alkynylene, alkenylene, acyl, aryl, heteroaryl, amide, oxime, ether, ester, triazole, —S—S, —CO—O—(CH₂)ₙ—S—S— (where n=2-6), —O—CO—O—(CH₂)ₙ—S—S— (where n=2-6), —S—CO—O—(CH₂)ₙ—S—S— (where n=2-6), —NH—CO—O—(CH₂)ₙ—S—S— (where n=2-6), carboxylate, carbonate, carbamate, urea, thiourea, polyethylene glycol (PEG) (e.g., PEGₙ, where n=1-200), polyproline, oligo-(4-piperidine) carboxylic acid, oligo piperidine, amino acid (e.g., hydrophilic amino acid), peptide, saccharo-peptide, sugar, peptidoglycan (e.g., an unnatural peptidoglycan), a polyvinylpyrrolidone, pluronic F-127, or any combination of two or more of the foregoing.

The first linker, the second linker, or both the first linker and the second linker can comprise PEG.

As indicated above, various substituents of the various formulae are “optionally substituted.” The term “substituted” as used herein means that any one or more hydrogens on the designated atom or group is replaced with a selection from the indicated group of substituents, provided that the designated atom's normal valence is not exceeded, and that the substitution results in a stable compound. When a substituent is oxo (keto, i.e., =0), then 2 hydrogens on an atom are replaced. The present disclosure includes all isotopes (including radioisotopes) of atoms occurring in the present conjugates.

When the conjugates are further substituted, they can be so substituted at one or more available positions, typically 1 to 3 or 4 positions, by one or more suitable groups such as those disclosed herein. Suitable groups that can be present on a “substituted” group include, for example and without limitation, halogen; cyano; hydroxyl; nitro; azido; alkanoyl (such as a C1-6 alkanoyl group such as acyl or the like); carboxamido; alkyl groups (including cycloalkyl groups, having 1 to about 8 carbon atoms); alkenyl and alkynyl groups (including groups having one or more unsaturated linkages and from 2 to about 8 carbon atoms); alkoxy groups having one or more oxygen linkages and from 1 to about 8 carbon atoms; aryloxy such as phenoxy; alkylthio groups including those having one or more thioether linkages and from 1 to about 8 carbon atoms; alkylsulfinyl groups including those having one or more sulfinyl linkages and from 1 to about 8 carbon atoms; alkylsulfonyl groups including those having one or more sulfonyl linkages and from 1 to about 8 carbon atoms; aminoalkyl groups including groups having one or more N atoms and from 1 to about 8 carbon atoms.

As used herein, “alkyl” is includes both branched and straight-chain saturated aliphatic hydrocarbon groups, having the specified number of carbon atoms. Examples of alkyl include, but are not limited to, methyl, ethyl, n-propyl, i-propyl, n-butyl, s-butyl, t-butyl, n-pentyl, and s-pentyl. Preferred alkyl groups are C1-6 alkyl groups. Especially preferred alkyl groups can be methyl, ethyl, propyl, butyl, and 3-pentyl.

In general, the term “acyl” or “acyl substituent” refers to a derived by the removal of one or more hydroxyl groups from an oxoacid, including inorganic acids, and contains a double-bonded oxygen atom and an alkyl group.

The term “alkylene,” by itself or as part of another substituent means, unless otherwise stated, a divalent radical derived from an alkyl, as exemplified, but not limited to, —CH₂CH₂CH₂CH₂—. Typically, an alkyl (or alkylene) group will have from 1 to 24 carbon atoms.

The term “heteroalkyl” by itself or in combination with another term means, unless otherwise stated, a stable straight or branched chain, or combination(s) thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quarternized. The heteroatom(s) O, N, P, S, and Si may be placed at any interior position of the heteroalkyl group or at the position at which the alkyl group is attached to the remainder of the molecule. Examples include, without limitation, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH₂═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

Similarly, the term “heteroalkylene” by itself or as part of another substituent, means (unless otherwise stated) a divalent radical derived from heteroalkyl, as exemplified, but not limited by, —CH₂—CH₂—S—CH₂—CH₂ and —CH₂—S—CH₂—CH₂—NH—CH₂. For heteroalkylene groups, heteroatoms can also occupy either or both of the chain termini (e.g., alkyleneoxy, alkylenedioxy, alkyleneamino, alkylenediamino, and the like). Still further, for alkylene and heteroakylene linking groups, no orientation of the linking group is implied by the direction in which the formula of the linking group is written. For example, the formula —C(O)₂R′— represents both —C(O)₂R′— and —R′ C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

In view of the above, a CAR T-cell-rejuvenating conjugate is provided. The rejuvenating conjugate can comprise an agonist of TLR7, TLR8, or TLR7/8 conjugated via a linker (e.g., the second linker) with a targeting moiety that binds a CAR of the CAR T-cell with specificity, wherein the CAR T-cell-rejuvenating conjugate has the structure:

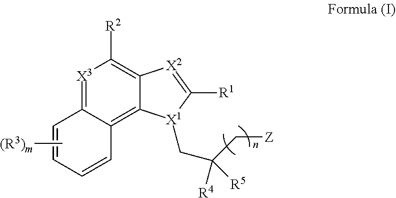

or is a pharmaceutically acceptable salt thereof, wherein:
   R¹ is an acyclic or cyclic alkyl, which is optionally substituted with one or more substituents independently selected from an alkyl, halo, heteroalkyl, alkoxy, and cycloalkyl;
   R² is —NR^{2x}R^{2y}, H, —OR^{z}, —SO₂N(R^{z})₂, or N₃, wherein:
      R^{2x} and R^{2y} are each independently hydrogen, —N(R^{z})₂, —CON(R^{z})₂, —C(R^{z})₂—N(R^{z})₂, —CS—N(R^{z})₂, or an alkyl, which is optionally substituted with one or more substituents independently selected from oxo, halo, alkyl, heteroalkyl, alkoxy, and cycloalkyl;
      each R^{z} is independently H or alkyl that is optionally substituted; or
      R^{2x} and R^{2y} are taken together to form a 3-10 membered mono- or bicyclic heterocycloalkyl, which is optionally substituted;
   each R³ is independently hydrogen, halo, —N₃, —CN, —NO₂, alkyl, heteroalkyl, cycloalkyl, heterocycloalkyl, alkoxy, aryl, heteroaryl, heterocycloalkyl, amino, hydroxy, carbonyl, or thiol, wherein the alkyl, heteroalkyl, cycloalkyl, heterocycloalkyl, or alkoxy is optionally substituted;
   R⁴ and R⁵ are each independently alkyl, alkoxy, halo, or cycloalkyl, wherein the alkyl, alkoxy, or cycloalkyl is optionally substituted;
   X¹, X², and X³ are independently N or CR^{q}, wherein each R^{q} is independently H, halo, or optionally substituted alkyl;
   Z is G-L-, wherein L is a linker (e.g., the second linker) and G is a targeting moiety (e.g., the first targeting moiety, the second targeting moiety, or both the first and second targeting moieties);
   n is 1-6; and
   m is 0-4.

In certain embodiments, the rejuvenating conjugate comprises an agonist of TLR7, TLR8, or TLR7/8 conjugated via a linker (e.g., the second linker) with a targeting moiety that binds (e.g., with specificity) a CAR of the CAR T-cell, wherein the CAR T-cell-rejuvenating conjugate has the structure:

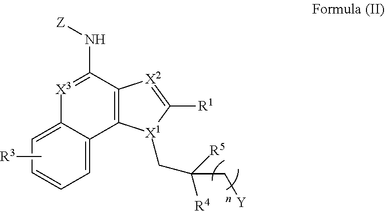

or is a pharmaceutically acceptable salt thereof, wherein:
   R¹, R³, R⁴, and R⁵ are each independently hydrogen, alkyl, alkoxyl, alkenyl, alkynyl, alicyclic, aryl, biaryl, halo, heteroaryl, —COR^{2x}

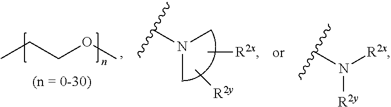

wherein each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl;
   Z is G-L-, wherein L is a linker (e.g., the second linker) and G is a targeting moiety (e.g., the first and/or second targeting moieties);
   X¹, X², and X³ are independently CR^{q} or N, wherein each R^{q} is independently hydrogen, halo, or optionally substituted alkyl;
   Y is H, —OR^{z}, —NR^{2x}R^{2y}, —SR^{z}, —SOR^{z}, —SO₃R^{z}, —N₃, —COR^{z}, —COOR^{z}, —CON(R^{z})₂, —COSR^{z}, —SO₂N(R^{z})₂, or —CON(R^{z})₂, wherein:
      R^{2x} and R^{2y} are each independently H, —N(R^{z})₂, —CON(R^{z})₂, —C(R^{z})₂—N(R^{z})₂, —CS—N(R^{z})₂, or optionally substituted alkyl (e.g., optionally substituted with one or more substituent, each substituent independently being oxo, halogen, alkyl, heteroalkyl, alkoxy, or cycloalkyl);
      each R^{z} is independently hydrogen or optionally substituted alkyl; or
      R^{2x} and R^{2y} are taken together to form an optionally substituted heterocycloalkyl (e.g., wherein the optionally substituted heterocycloalkyl is a mono- or bicyclic heterocycloalkyl and/or wherein the optionally substituted heterocycloalkyl is a 3-10 membered heterocycloalkyl); and
   n in Formula (II) is 0-30.

In certain embodiments, the rejuvenating conjugate comprises an agonist of TLR7, TLR8, or TLR7/8 conjugated via a linker (e.g., the second linker) with a targeting moiety that binds (e.g., with specificity) a CAR of the CAR T-cell, wherein the CAR T-cell-rejuvenating conjugate has the structure:

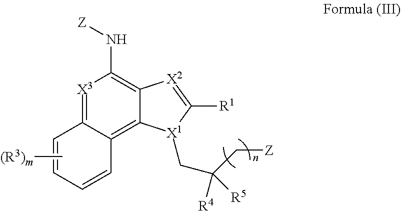

or is a pharmaceutically acceptable salt thereof, wherein:
   R¹ is an acyclic or cyclic alkyl, which is optionally substituted with one or more substituents independently selected from halo, alkyl, heteroalkyl, alkoxy, and cycloalkyl;
   Y is H, —OR^{z}, —NR^{2x}R^{2y}, —SR^{z}, —SOR^{z}, —SO₃R^{z}, —N₃, —COR^{z}, —COOR^{z}, —CONR^{z}₂, —COSR^{z}, —SO₂N(R^{z})₂, or —CON(R^{z})₂, wherein
      each R^{z} is independently H or optionally substituted alkyl;
      R^{2x} and R^{2y} are each independently H, —N(R^{z})₂, —CON(R^{z})₂, —C(R^{z})₂—N(R^{z})₂, —CS—N(R^{Z})₂, or alkyl, which is optionally substituted with one or more substituents independently selected from oxo, halo, alkyl, heteroalkyl, alkoxy, and cycloalkyl; or
      R^{2x} and R^{2y} are taken together to form a 3-10 membered mono- or bicyclic heterocycloalkyl, which is optionally substituted;
   each R³ is independently halo, —N₃, —CN, —NO₂, alkyl, heteroalkyl, cycloalkyl, heterocycloalkyl, alkoxy, aryl, heteroaryl, heterocycloalkyl, amino, hydroxy, carbonyl, or thiol, wherein the alkyl, alkoxy, heteroalkyl, cycloalkyl, or heterocycloalkyl is optionally substituted;
   R⁴ and R⁵ are each independently alkyl, alkoxy, halogen, or cycloalkyl, wherein the alkyl, alkoxy, or cycloalkyl is optionally substituted;
   X¹, X², and X³ are independently CR^{q} or N, wherein each R^{q} is independently H, halogen, or optionally substituted alkyl;
   Z is L-G, wherein L is a linker (e.g., the second linker) and G is a targeting moiety (e.g., the first and/or second targeting moieties);
   n is 0-30; and
   m is 0-4.

G (the targeting moiety) in Formula (I), Formula (II) or Formula (III) can be a group or comprise a group with the structure:

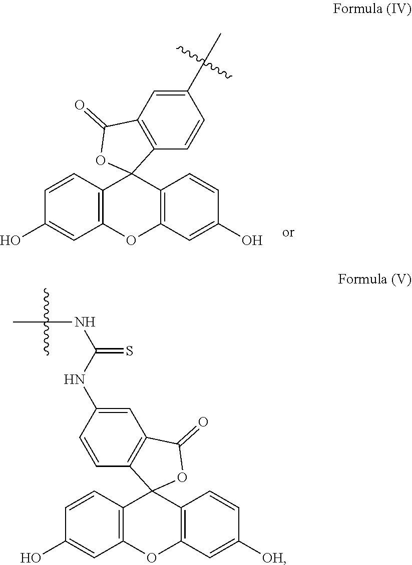 [SMILES: *c1ccc2c(c1)C(=O)OC21c2ccc(O)cc2Oc2cc(O)ccc21 ; *NC(=S)Nc1ccc2c(c1)C(=O)OC21c2ccc(O)cc2Oc2cc(O)ccc21]

or a pharmaceutically acceptable salt of either of the foregoing structures. Each of X¹, X², and X³ of Formula (I), (II), and/or (III) can be nitrogen (N).

The CAR T-cell-rejuvenating conjugate can have the structure:

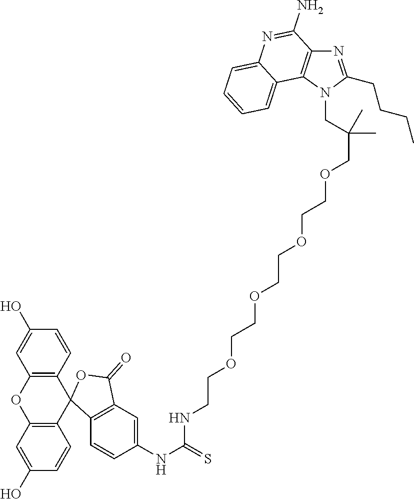 [SMILES: CCCCc1nc2c(N)nc3ccccc3c2n1CC(C)(C)COCCOCCOCCOCCNC(=S)Nc1ccc2c(c1)C(=O)OC21c2ccc(O)cc2Oc2cc(O)ccc21]

or a pharmaceutically acceptable salt thereof.

Alternatively, the CAR T-cell-rejuvenating conjugate can have a structure selected from:

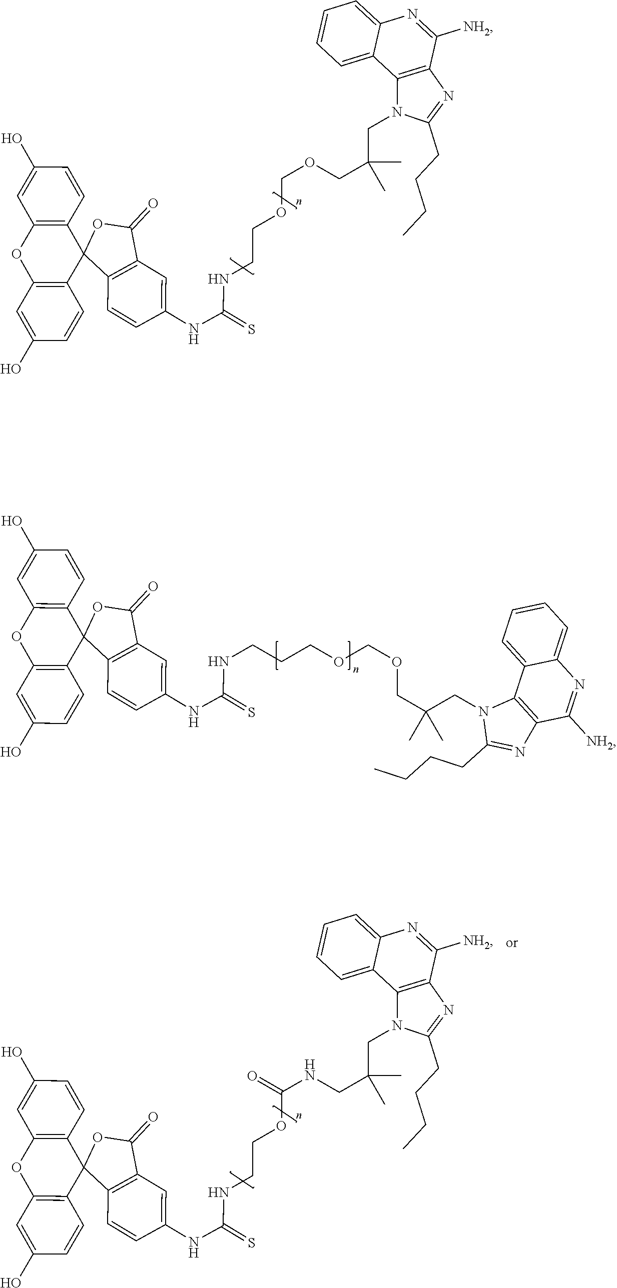

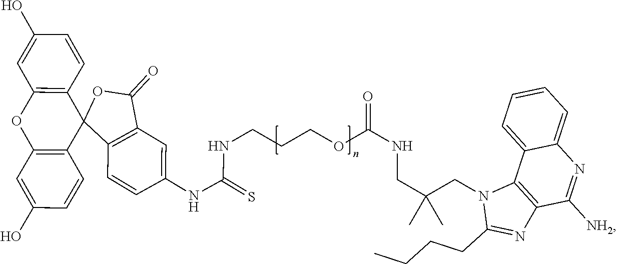

wherein n=0-200 or can be a pharmaceutically acceptable salt of any of the foregoing structures. Further alternatively, the CAR T-cell-rejuvenating conjugate can have a structure selected from:

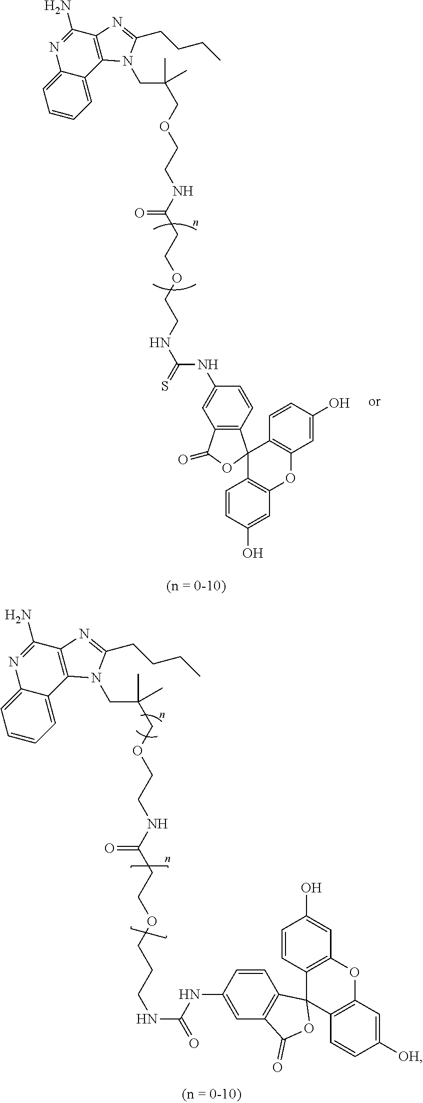

or be a pharmaceutically acceptable salt of either of the foregoing structures. In certain embodiments, the CAR T-cell-rejuvenating conjugate can have a structure selected from:

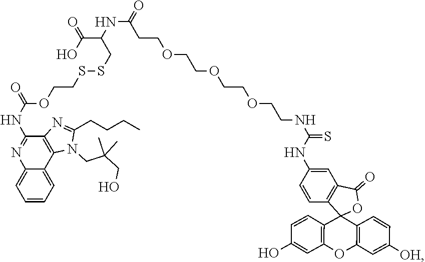 [SMILES: CCCCc1nc2c(NC(=O)OCCSSCC(NC(=O)CCOCCOCCOCCNC(=S)Nc3ccc4c(c3)C(=O)OC43c4ccc(O)cc4Oc4cc(O)ccc43)C(=O)O)nc3ccccc3c2n1CC(C)(C)CO]

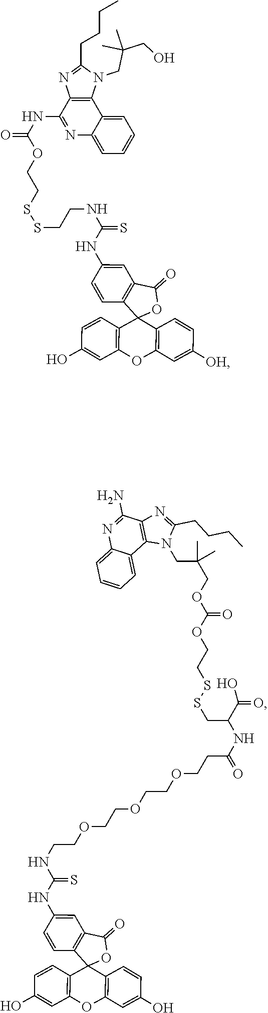 [SMILES: CCCCc1nc2c(NC(=O)OCCSSCCNC(=S)Nc3ccc4c(c3)C(=O)OC43c4ccc(O)cc4Oc4cc(O)ccc43)nc3ccccc3c2n1CC(C)(C)CO ; CCCCc1nc2c(N)nc3ccccc3c2n1CC(C)(C)COC(=O)OCCSSCC(NC(=O)CCOCCOCCOCCNC(=S)Nc1ccc2c(c1)C(=O)OC21c2ccc(O)cc2Oc2cc(O)ccc21)C(=O)O]

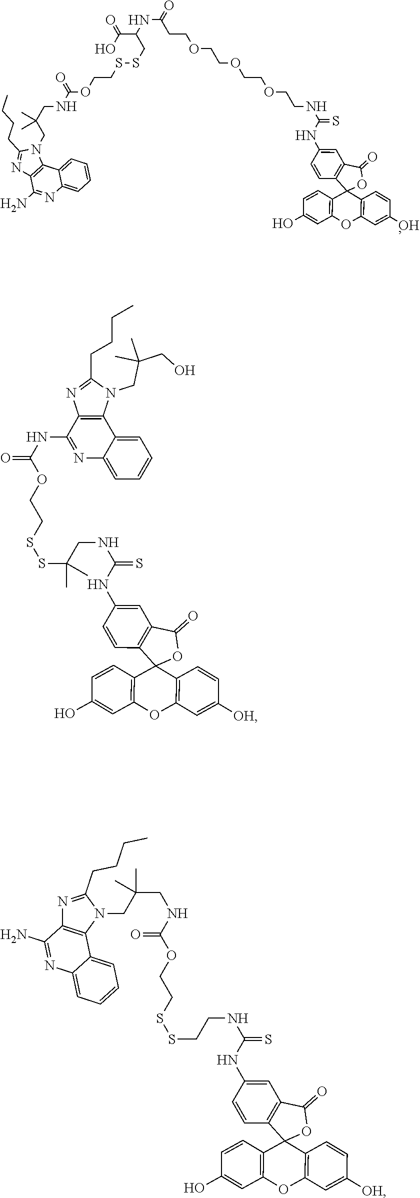 [SMILES: CCCCc1nc2c(N)nc3ccccc3c2n1CC(C)(C)CNC(=O)OCCSSCC(NC(=O)CCOCCOCCOCCNC(=S)Nc1ccc2c(c1)C(=O)OC21c2ccc(O)cc2Oc2cc(O)ccc21)C(=O)O ; CCCCc1nc2c(NC(=O)OCCSSC(C)(C)CNC(=S)Nc3ccc4c(c3)C(=O)OC43c4ccc(O)cc4Oc4cc(O)ccc43)nc3ccccc3c2n1CC(C)(C)CO ; CCCCc1nc2c(N)nc3ccccc3c2n1CC(C)(C)CNC(=O)OCCSSCCNC(=S)Nc1ccc2c(c1)C(=O)OC21c2ccc(O)cc2Oc2cc(O)ccc21]

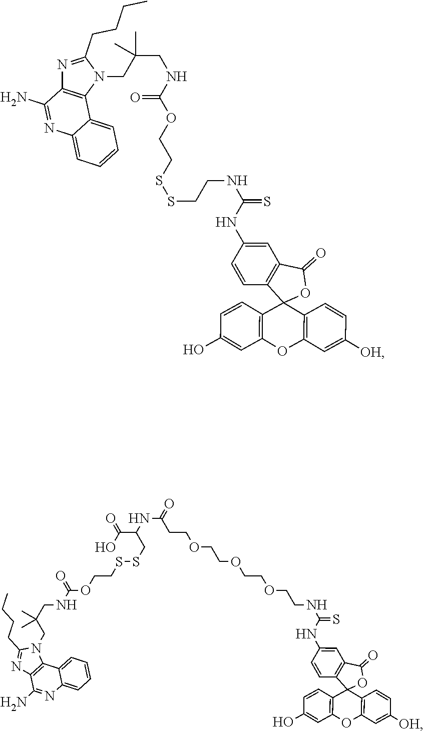 [SMILES: CCCCc1nc2c(N)nc3ccccc3c2n1CC(C)(C)CNC(=O)OCCSSCCNC(=S)Nc1ccc2c(c1)C(=O)OC21c2ccc(O)cc2Oc2cc(O)ccc21 ; CCCCc1nc2c(N)nc3ccccc3c2n1CC(C)(C)CNC(=O)OCCSSCC(NC(=O)CCOCCOCCOCCNC(=S)Nc1ccc2c(c1)C(=O)OC21c2ccc(O)cc2Oc2cc(O)ccc21)C(=O)O]

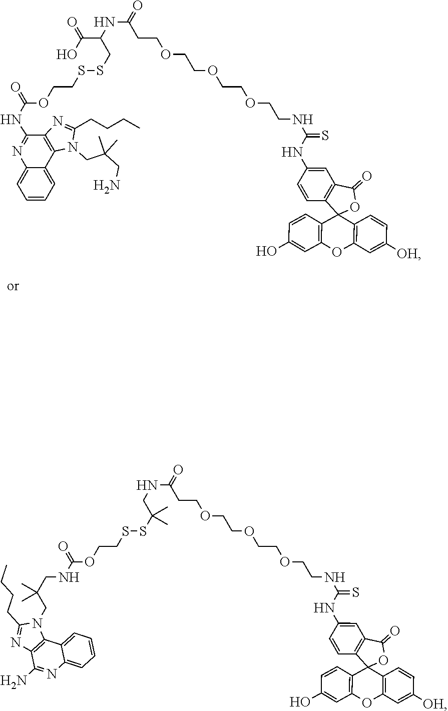 [SMILES: CCCCc1nc2c(NC(=O)OCCSSCC(NC(=O)CCOCCOCCOCCNC(=S)Nc3ccc4c(c3)C(=O)OC43c4ccc(O)cc4Oc4cc(O)ccc43)C(=O)O)nc3ccccc3c2n1CC(C)(C)CN ; CCCCc1nc2c(N)nc3ccccc3c2n1CC(C)(C)CNC(=O)OCCSSC(C)(C)CNC(=O)CCOCCOCCOCCNC(=S)Nc1ccc2c(c1)C(=O)OC21c2ccc(O)cc2Oc2cc(O)ccc21]

or can be a pharmaceutically acceptable salt of any of the foregoing structures. Other structures for the CAR T-cell rejuvenating conjugate can include:

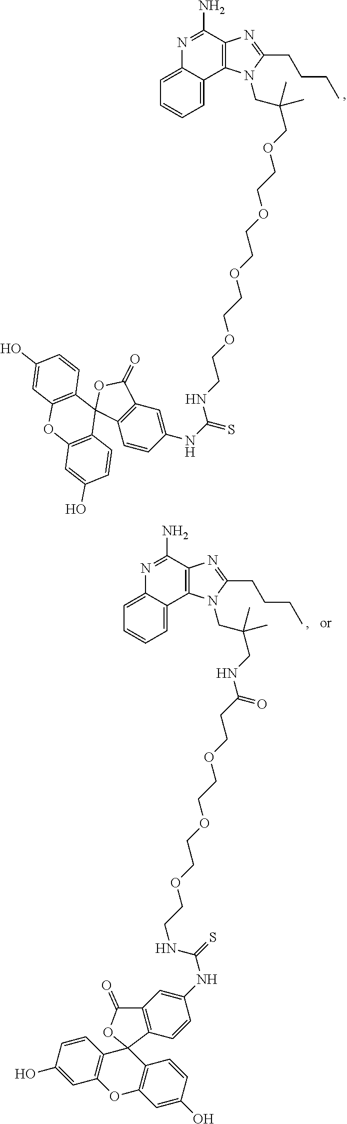 [SMILES: CCCCc1nc2c(N)nc3ccccc3c2n1CC(C)(C)COCCOCCOCCOCCNC(=S)Nc1ccc2c(c1)C(=O)OC21c2ccc(O)cc2Oc2cc(O)ccc21 ; CCCCc1nc2c(N)nc3ccccc3c2n1CC(C)(C)CNC(=O)CCOCCOCCOCCNC(=S)Nc1ccc2c(c1)C(=O)OC21c2ccc(O)cc2Oc2cc(O)ccc21]

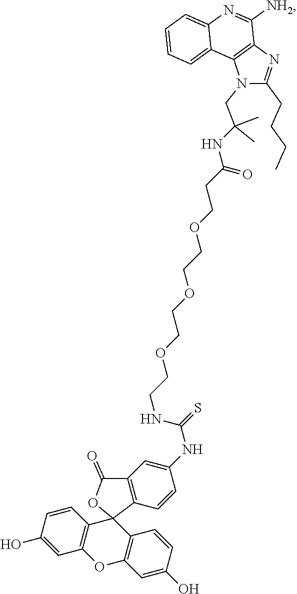 [SMILES: CCCCc1nc2c(N)nc3ccccc3c2n1CC(C)(C)NC(=O)CCOCCOCCOCCNC(=S)Nc1ccc2c(c1)C(=O)OC21c2ccc(O)cc2Oc2cc(O)ccc21]

or a pharmaceutically acceptable salt of any of the foregoing. In certain embodiments, the CAR T-cell-rejuvenating conjugate can have a structure selected from the following:

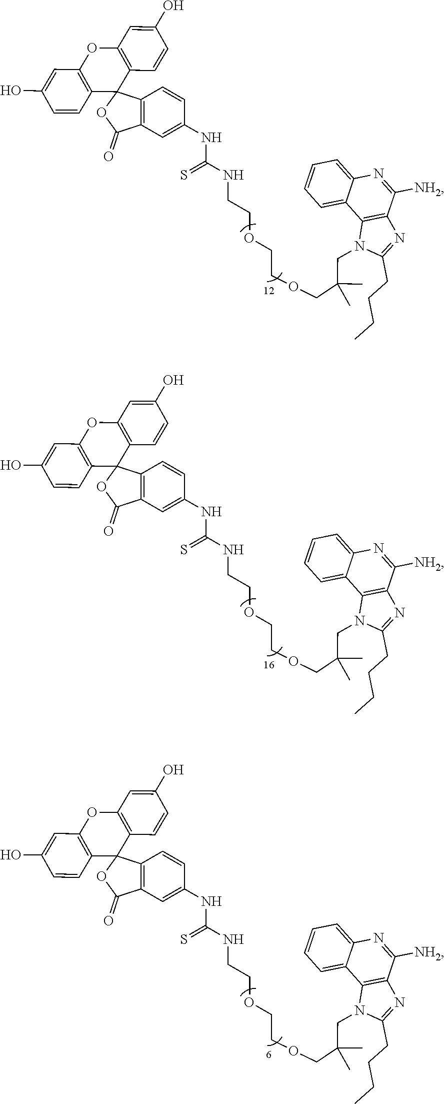

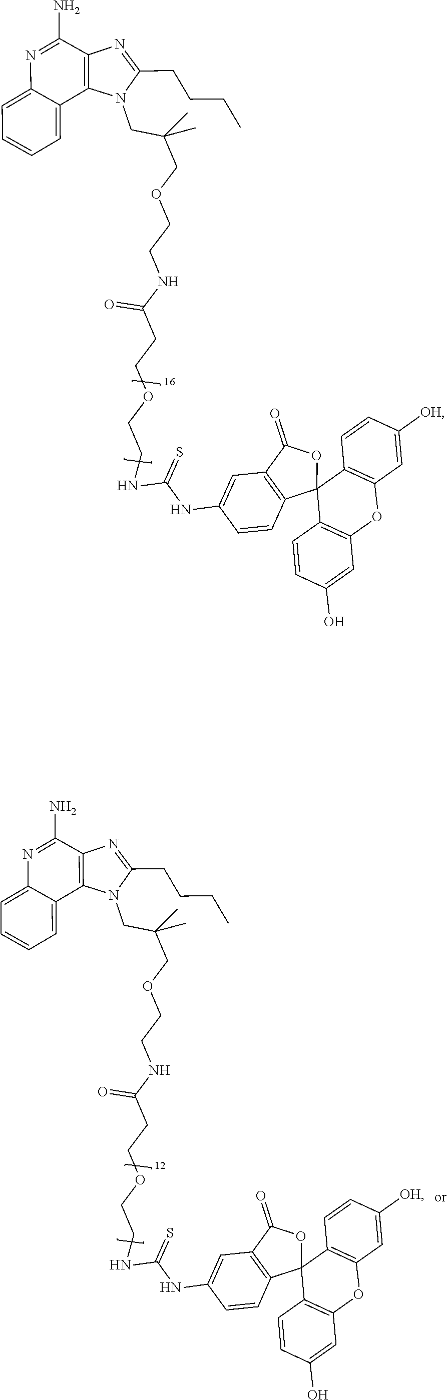

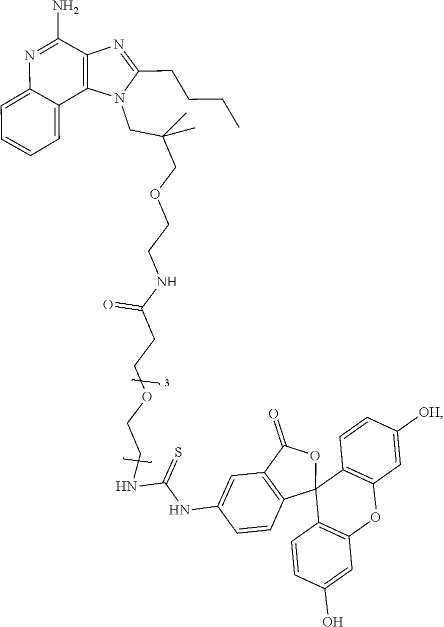

or can be a pharmaceutically acceptable salt of any of the foregoing structures.

In certain embodiments, the CAR T-cell-rejuvenating conjugate can include:

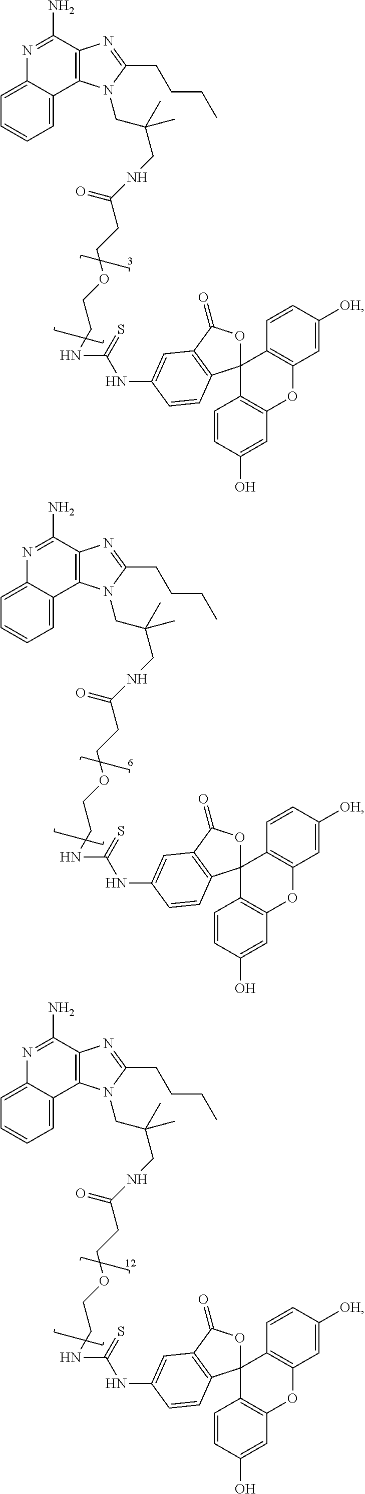

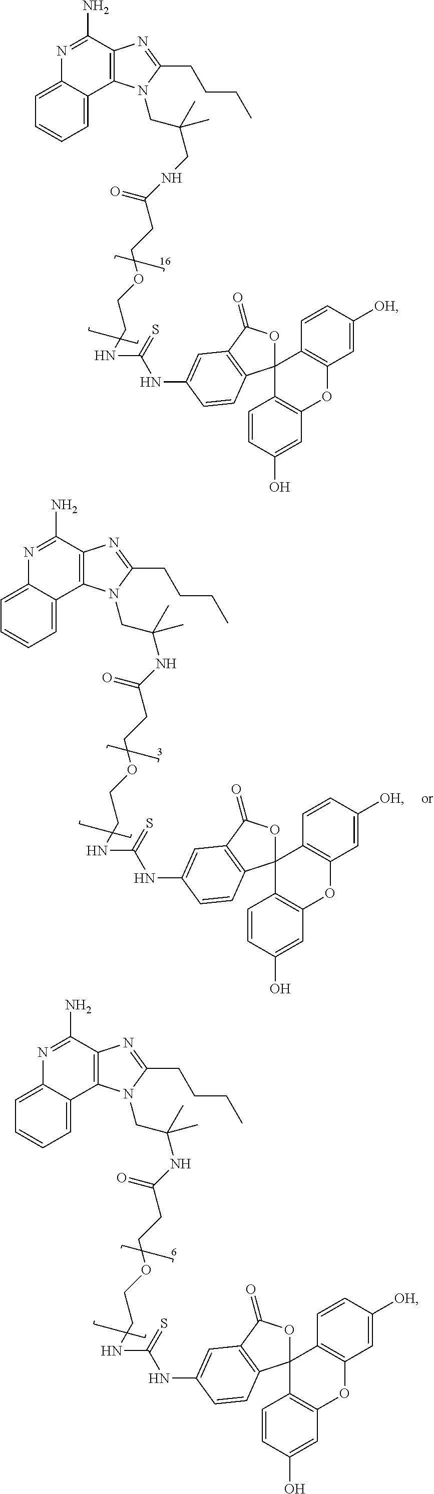

or a pharmaceutically acceptable salt of any of the foregoing structures.

As noted above, the CAR T-cell-rejuvenating conjugate can comprise at least one targeting moiety (e.g., to target a CAR). The targeting moiety with regard to Formulae (I)-(III) can be selected from the group consisting of DNP, L-rhamnose, FK506, TNP, biotin, rapamycin, digoxigenin, folate, 5-methyl tetrahydrofolate, fluorescein, FITC, NHS-fluorescein, pentafluorophenyl ester, tetrafluorophenyl ester, knottin, centyrin, and DARPin.

Further embodiments can comprise a linker disposed between the targeting moiety and the active agent (i.e., agonist of TLR7, TLR8, or TLR7/8). The term “linker” with regard to Formulae (I)-(III) can include a chain of atoms that is bio-functionally adapted to form a chemical bond with the agonist of TLR7, TLR8, or TLR7/8 conjugated with either the first targeting moiety or the second targeting moiety and connects two or more parts of a molecule to form a compound. Illustratively, the chain of atoms can be selected from carbon (C), nitrogen (N), oxygen (O), sulfur (S), silicon (Si), and phosphorus (P), or C, N, O, S, and P, C, N, O, and S. The chain of atoms can covalently connect different functional capabilities, such as the small molecule ligand and a targeting moiety of the conjugate. The linker (e.g., the first or second linker) can comprise a wide variety of links, such as in the range from about 2 to about 2,000 atoms in the contiguous backbone and can comprise a releasable or non-releasable linker. In certain embodiments the linker can be optional, if so desired.

The linker can comprise C₁-C₂₀ alkyl, alkylene, heteroalkylene, —O-alkynylene, alkenylene, acyl, aryl, heteroaryl, amide, oxime, ether, ester, triazole, —S—S, —CO—O—(CH₂)ₙ—S—S— (where n=2-6), —O—CO—O—(CH₂)ₙ—S—S— (where n=2-6), —S—CO—O—(CH₂)ₙ—S—S— (where n=2-6), —NH—CO—O—(CH₂)ₙ—S—S— (where n=2-6), carboxylate, carbonate, carbamate, urea, thiourea, PEG (e.g., PEGₙ, where n=1-200), polyproline, oligo-(4-piperidine) carboxylic acid, oligo piperidine, amino acid (e.g., hydrophilic amino acid), peptide, saccharo-peptide, sugar, peptidoglycan (e.g., an unnatural peptidoglycan), a polyvinylpyrrolidone, pluronic F-127, or any combination of two or more of the foregoing.

The linker can comprise PEG. By way of example, the linker can comprise a structure having the formula:

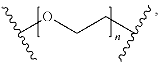

wherein n is an integer from 0 to 200. In another embodiment, n can be an integer from 0 to 150, 0 to 110, 0 to 100, 0 to 90, 0 to 80, 0 to 70, 0 to 60, 0 to 50, 0 to 40, 0 to 30, 0 to 20, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, 0 to 1, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 15 to 31, 15 to 32, 15 to 33, 15 to 34, 15 to 35, 15 to 36, 15 to 37, 15 to 38, 15 to 39, 15 to 40, 15 to 50, 15 to 60, 15 to 70, 15 to 80, 15 to 90, 15 to 100, 15 to 110, 15 to 120, 15 to 130, 15 to 140, 15 to 150, or n can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, 100, 108, 110, 120, 130, 140, or 150.

The linker can be a direct linkage (e.g., a reaction between the isothiocyanate group of FIC and a free amine group of a ligand) or the linkage can be through an intermediary linker. In one embodiment, if present, an intermediary linker can be any biocompatible linker known in the art, such as a divalent linker. In one illustrative embodiment, the linker can be divalent and comprise about 1 to about 30 carbon atoms (such as 1-30 carbon atoms). In another illustrative embodiment, the divalent linker can comprise about 2 to about 20 carbon atoms (such as 2-20 carbon atoms). In other embodiments, lower molecular weight divalent linkers (i.e., those having an approximate molecular weight of about 30 Daltons to about 300 Daltons) are employed. In another embodiment, linker lengths that are suitable include, without limitation, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

Examples of divalent linkers are shown in Table 1, in which (*) indicates the point of attachment to a ligand, a targeting moiety, or a rejuvenating compound (e.g., a TLR7/8 agonist).

TABLE 1
Linker Formulae

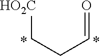

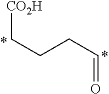

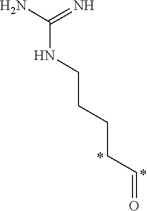

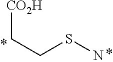

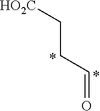

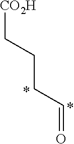

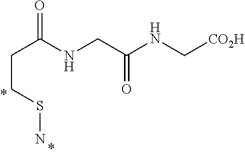 [SMILES: NSCCC(=O)NCC(=O)NCC(=O)O]

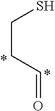

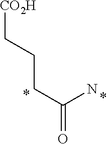

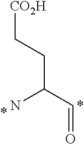

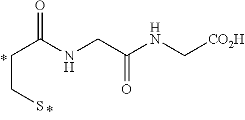 [SMILES: O=C(O)CNC(=O)CNC(=O)CCS]

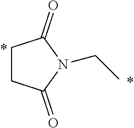

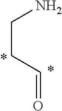

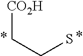

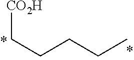

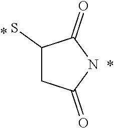

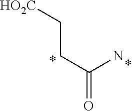

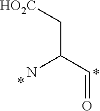

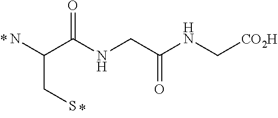 [SMILES: NC(CS)C(=O)NCC(=O)NCC(=O)O]

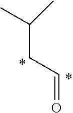

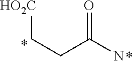

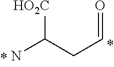

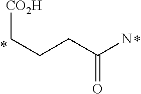

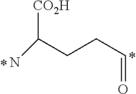

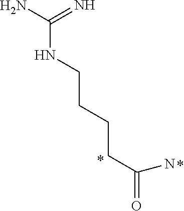 [SMILES: N=C(N)NCCCCC(N)=O]

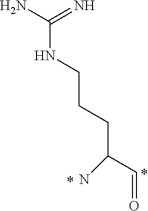 [SMILES: N=C(N)NCCCC(N)C=O]

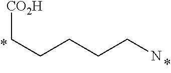

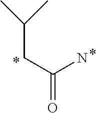

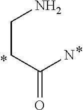

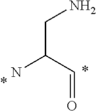

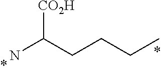

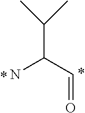

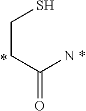

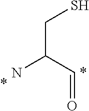

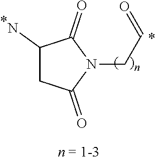

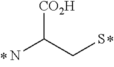

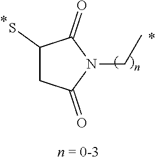

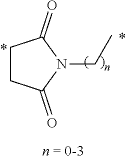

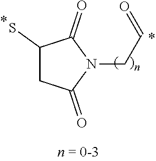

Other structures for linker moieties can include:

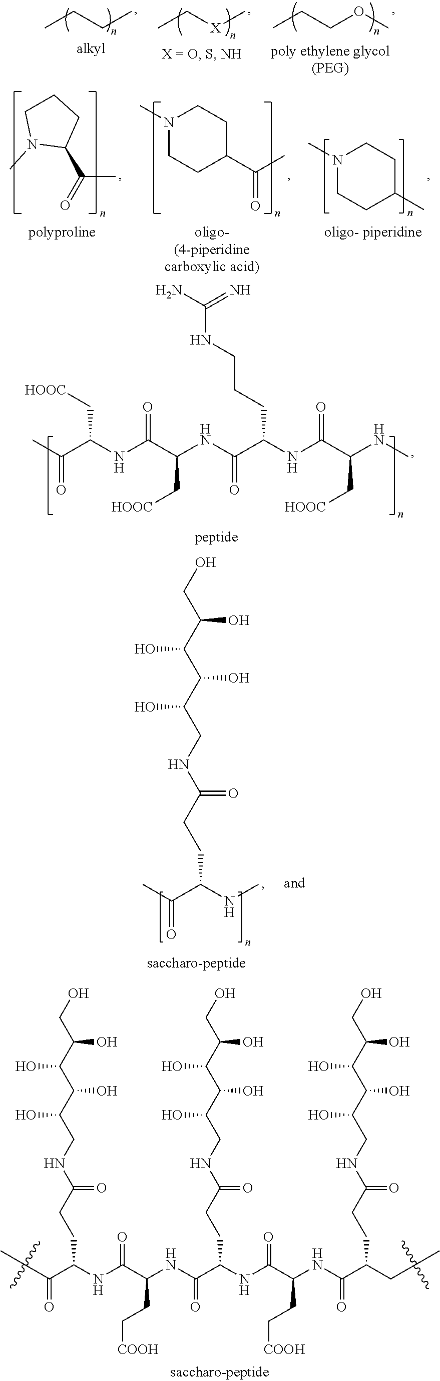

wherein n is an integer from 0 to 200.

The linker can be releasable or non-releasable. The term “releasable” in the context of a linker means a linker that includes at least one bond that can be broken (e.g., chemically or enzymatically hydrolyzed) under physiological conditions, such as, for example, by reducing agent-labile, pH-labile, acid-labile, base-labile, oxidatively labile, metabolically labile, biochemically labile, enzyme-labile or p-aminobenzylic based multivalent releasable bond. It is appreciated that the physiological conditions resulting in bond breaking do not necessarily include a biological or metabolic process and instead can include a standard chemical reaction, such as a hydrolysis reaction for example, at physiological pH or as a result of compartmentalization into a cellular organelle, such as an endosome, having a lower pH than cytosolic pH. A cleavable bond can connect two adjacent atoms within the releasable linker and/or connect other linker portions or the targeting moiety and/or active component (e.g., the agonist of TLR7, TLR8, or TLR7/8), as described herein, for example, at either or both ends of the releasable linker. In some instances, the releasable linker is broken into two or more fragments. In some instances, the releasable linker is separated from the targeting moiety. In certain embodiments, a CAR T-cell-rejuvenating conjugate that comprises a releasable linker will, when administered, result in the targeting moiety and agonist being released from each other on or about the conjugate enters the CAR-expressing cell.

In contrast, the term “non-releasable” in the context of a linker means a linker that includes at least one bond that is not easily or quickly broken under physiological conditions. In some embodiments, a non-releasable linker comprises a backbone that is stable under physiological conditions (e.g., the backbone is not susceptible to hydrolysis (e.g., aqueous hydrolysis or enzymatic hydrolysis)). In some embodiments, agonist of TLR7, TLR8, or TLR78 of the CAR T-cell rejuvenating conjugate that comprises a non-releasable linker does not release from the targeting moiety. In some embodiments, the non-releasable linker lacks a disulfide bond (e.g., S—S) or an ester in the backbone. In some embodiments, the conjugates comprise a targeting moiety and an active component (e.g., the agonist) connected by a backbone that is substantially stable for the entire duration of the conjugates's circulation. The non-releasable linker can comprise: an amide, ester, ether, amine, and/or thioether (e.g., thio-maleimide). While specific examples are provided herein, it will be understood that any molecule(s) can be used in the non-releasable linker provided that at least one bond that is not easily or quickly broken under physiological conditions is formed.

Both releasable and non-releasable linkers can be engineered to optimize biodistribution, bioavailability, and PK/PD (e.g., of the respective conjugate and/or active component thereof) and/or to increase uptake (e.g., of the respective conjugate and/or active component thereof) into a targeted cell pursuant to methodologies commonly known in the art or hereinafter developed such as through PEGlaytion and the like.

The conjugates, and pharmaceutically acceptable salts thereof, can be prepared by conventional methods of organic synthesis practiced by those skilled in the art. Descriptions of conjugates are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl can be attached to the remainder of the molecule via a ring heteroatom in compliance with principles of chemical bonding known to those skilled in the art, thereby avoiding inherently unstable compounds.

The conjugates, and pharmaceutically acceptable salts thereof, can exist as geometric isomers. Accordingly, various embodiments can include pure geometric isomers or mixtures of geometric isomers of the conjugates. The conjugates, and pharmaceutically acceptable salts thereof, can also exist in unsolvated and solvated forms, including hydrated forms. In general, the solvated forms are equivalent to unsolvated forms and are encompassed within the scope of the present disclosure.

The term “pharmaceutically acceptable salt” refers to those salts whose counter ions may be used in pharmaceuticals. In various embodiments, such salts include, but are not limited to 1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or 2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like. Pharmaceutically acceptable salts are well-known to those skilled in the art, and any such pharmaceutically acceptable salt is contemplated in connection with the embodiments described herein.

In various embodiments, suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

In various embodiments, suitable base salts are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemisalts of acids and bases also can be formed, for example, hemisulphate and hemicalcium salts.

In view of the above, further provided is a pharmaceutical composition. In certain embodiments, the pharmaceutical composition comprises a CAR T-cell-rejuvenating conjugate and a pharmaceutically acceptable carrier.

As used herein, the term “composition” generally refers to any product comprising more than one ingredient, including the conjugates described herein. The compositions can be prepared from isolated compounds (i.e., conjugates; “compounds” and “conjugates” are used interchangeably herein) or from salts, solutions, hydrates, solvates, and other forms of the CAR T-cell rejuvenating conjugates. Certain functional groups, such as the hydroxy, amino, and like groups, can form complexes with water and/or various solvents, in the various physical forms of the compounds. It will also be understood that, in certain circumstances, the compounds (and compositions comprising the compounds) can be prepared from various amorphous, non-amorphous, partially crystalline, crystalline, and/or other morphological forms of the compounds, and the compositions can be prepared from various hydrates and/or solvates of the compounds. Accordingly, pharmaceutical compositions that recite the conjugates include each of, or any combination of, or individual forms of, the various morphological forms and/or solvate or hydrate forms of the compounds.

Compounds and compositions can be administered in unit dosage forms and/or compositions containing one or more pharmaceutically acceptable carriers, adjuvants, diluents, excipients, and/or vehicles, and combinations thereof. The term “administering,” and its formatives, generally refer to any and all means of introducing the compounds and compositions described herein to a cell, tissue, organ, or biological fluid of a subject.

As used herein, a “subject” is a mammal, preferably a human, but it can also be a non-human animal (including, without limitation, a laboratory, an agricultural, a domestic, or a wild animal). Thus, the methods, compounds, and compositions are applicable to both human and veterinary disease and applications. In various aspects, the subject can be a laboratory animal such as a rodent (e.g., mouse, rat, hamster, etc.), a rabbit, a monkey, a chimpanzee, a domestic animal such as a dog, a cat, or a rabbit, an agricultural animal such as a cow, a horse, a pig, a sheep, or a goat, or a wild animal in captivity such as a bear, a panda, a lion, a tiger, a leopard, an elephant, a zebra, a giraffe, a gorilla, a dolphin, or a whale. In certain embodiments, subjects are “patients,” i.e., living humans or animals that are receiving medical care for a disease or condition, which includes persons or animals with no defined illness who are being evaluated for signs of pathology. In certain embodiments, subjects that can be addressed using the methods hereof include subjects identified or selected as having or being at risk for having cancer. Such identification and/or selection can be made by clinical or diagnostic evaluation.

The compounds can be formulated as pharmaceutical compositions and/or administered to a subject, such as a human patient, in a variety of forms adapted to the chosen route of administration. Indeed, any suitable method of administration known in the art can be used. In one aspect, the compound, or the pharmaceutically acceptable salt thereof, or a pharmaceutical composition comprising the compound and a pharmaceutically acceptable carrier, can be administered in unit dosage forms and/or formulations containing conventional nontoxic pharmaceutically acceptable carriers, adjuvants, and vehicles.

Further, the compound, or the pharmaceutically acceptable salt thereof, can be administered directly into the blood stream, into muscle, or into an internal organ. In various embodiments, suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. In one embodiment, means for parenteral administration include needle (including microneedle) injectors, needle-free injectors and infusion techniques. The compounds and compositions hereof can be formulated for the desired administration modality as well.

For example, parenteral formulations are typically aqueous solutions and can contain carriers or excipients such as salts, carbohydrates and buffering agents (preferably at a pH from 3 to 9), but can also be formulated, where suitable, as a sterile non-aqueous solution or as a dried form to be used in conjunction with a suitable vehicle such as sterile, pyrogen-free water or sterile saline. In other embodiments, any of the liquid formulations described herein can be adapted for parenteral administration. The preparation under sterile conditions, by lyophilization to produce a sterile lyophilized powder for a parenteral formulation, can readily be accomplished using standard pharmaceutical techniques well-known to those skilled in the art. The solubility of the compound, or the pharmaceutically acceptable salt thereof, used in the preparation of a parenteral formulation can be increased by the use of appropriate formulation techniques, such as the incorporation of solubility-enhancing agents.

The pharmaceutical dosage forms of the compound that are suitable for injection or infusion can include sterile aqueous solutions or dispersions or sterile powders comprising the active ingredients that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example and without limitation, water, ethanol, a polyol (e.g., glycerol, propylene glycol, liquid PEG(s), and the like), vegetable oils, nontoxic glyceryl esters, and/or suitable mixtures thereof. In at least one embodiment, the proper fluidity can be maintained by the formation of liposomes, by the maintenance of the required particle size in the case of dispersions or by the use of surfactants. The action of microorganisms can be prevented by the addition of various antibacterial and antifungal agents, such as parabens, chlorobutanol, phenol, sorbic acid, thimerosal, and the like. In certain cases, it can be desirable to include one or more isotonic agents, such as sugars, buffers, or sodium chloride. Prolonged absorption of the injectable compositions can be brought about by the incorporation of agents formulated to delay absorption, for example, aluminum monostearate and gelatin.

Sterile injectable solutions can be prepared by incorporating the active component in the required amount of the appropriate solvent with one or more of the other ingredients set forth above, as required, followed by filter sterilization. In the case of sterile powders for the preparation of sterile injectable solutions, the preferred methods of preparations are vacuum drying and the freeze-drying techniques, which yield a powder of the active ingredient plus any additional desired ingredient present in the previously sterile-filtered solutions.

Useful dosages of the compounds can be determined by comparing their in vitro activity and the in vivo activity in animal models. Methods of the extrapolation of effective dosages in mice and other animals to human subjects are known in the art. Indeed, the dosage of the compound can vary significantly depending on the condition of the subject, the cancer type being treated, how advanced the pathology is, the route of administration of the compound and tissue distribution, and the possibility of co-usage of other therapeutic treatments (such as radiation therapy or additional drugs in combination therapies). The amount of the compositions and/or compound(s) required for use in treatment (e.g., the therapeutically or prophylactically effective amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise. “Therapeutically effective amount” or “prophylactically effective amount” is defined as an amount of a reagent or pharmaceutical composition that is sufficient to induce a desired response.

The amount of the compound, or the pharmaceutically acceptable salt thereof, to be administered to the subject can vary significantly depending on the cancer being treated, the route of administration of the compound, or the pharmaceutically acceptable salt thereof, and the tissue distribution. The amount to be administered to a subject can be based on body surface area, mass, and physician assessment.

In various embodiments, amounts to be administered can range, for example, from about 0.05 mg to about 30 mg, 0.05 mg to about 25.0 mg, about 0.05 mg to about 20.0 mg, about 0.05 mg to about 15.0 mg, about 0.05 mg to about 10.0 mg, about 0.05 mg to about 9.0 mg, about 0.05 mg to about 8.0 mg, about 0.05 mg to about 7.0 mg, about 0.05 mg to about 6.0 mg, about 0.05 mg to about 5.0 mg, about 0.05 mg to about 4.0 mg, about 0.05 mg to about 3.0 mg, about 0.05 mg to about 2.0 mg, about 0.05 mg to about 1.0 mg, about 0.05 mg to about 0.5 mg, about 0.05 mg to about 0.4 mg, about 0.05 mg to about 0.3 mg, about 0.05 mg to about 0.2 mg, about 0.05 mg to about 0.1 mg, about 0.01 mg to about 2 mg, about 0.3 mg to about 10 mg, about 0.1 mg to about 20 mg, or about 0.8 to about 3 mg. One of skill in the art will readily appreciate that the dose may vary within the various ranges provided above based on the factors noted above and may be at the physician's discretion.

In other embodiments, the dose of the compound, or the pharmaceutically acceptable salt thereof, can range, for example, from about 50 nmoles/kg to about 3,000 nmoles/kg of subject body weight, about 50 nmoles/kg to about 2,800 nmoles/kg about 50 nmoles/kg to about 2,600 nmoles/kg about 50 nmoles/kg to about 2,400 nmoles/kg about 50 nmoles/kg to about 2,200 nmoles/kg about 50 nmoles/kg to about 2,100 nmoles/kg about 50 nmoles/kg to about 2,000 nmoles/kg, about 50 nmoles/kg to about 1,000 nmoles/kg, about 50 nmoles/kg to about 900 nmoles/kg, about 50 nmoles/kg to about 800 nmoles/kg, about 50 nmoles/kg to about 700 nmoles/kg, about 50 nmoles/kg to about 600 nmoles/kg, about 50 nmoles/kg to about 500 nmoles/kg, about 50 nmoles/kg to about 400 nmoles/kg, about 50 nmoles/kg to about 300 nmoles/kg, about 50 nmoles/kg to about 200 nmoles/kg, about 50 nmoles/kg to about 100 nmoles/kg, about 100 nmoles/kg to about 300 nmoles/kg, about 100 nmoles/kg to about 500 nmoles/kg, about 100 nmoles/kg to about 1,000 nmoles/kg, about 100 nmoles/kg to about 2,000 nmoles/kg of subject body weight. In other embodiments, the dose may be about 1 nmoles/kg, about 5 nmoles/kg, about 10 nmoles/kg, about 20 nmoles kg, about 25 nmoles/kg, about 30 nmoles/kg, about 40 nmoles/kg, about 50 nmoles/kg, about 60 nmoles/kg, about 70 nmoles/kg, about 80 nmoles/kg, about 90 nmoles/kg, about 100 nmoles/kg, about 150 nmoles/kg, about 200 nmoles/kg, about 250 nmoles/kg, about 300 nmoles/kg, about 350 nmoles/kg, about 400 nmoles/kg, about 450 nmoles/kg, about 500 nmoles/kg, about 600 nmoles/kg, about 700 nmoles/kg, about 800 nmoles/kg, about 900 nmoles/kg, about 1000 nmoles/kg, about 2,000 nmoles/kg, about 2,500 nmoles/kg or about 3,000 nmoles/kg of body weight of the subject. In yet other embodiments, the dose may be about 0.1 nmoles/kg, about 0.2 nmoles/kg, about 0.3 nmoles/kg, about 0.4 nmoles kg, or about 0.5 nmoles/kg, about 0.1 nmoles/kg to about 1000 nmoles/kg, about 0.1 nmoles/kg to about 900 nmoles/kg, about 0.1 nmoles/kg to about 850 nmoles/kg, about 0.1 nmoles/kg to about 800 nmoles/kg, about 0.1 nmoles/kg to about 700 nmoles/kg, about 0.1 nmoles/kg to about 600 nmoles/kg, about 0.1 nmoles/kg to about 500 nmoles/kg, about 0.1 nmoles/kg to about 400 nmoles/kg, about 0.1 nmoles/kg to about 300 nmoles/kg, about 0.1 nmoles/kg to about 200 nmoles/kg, about 0.1 nmoles/kg to about 100 nmoles/kg, about 0.1 nmoles/kg to about 50 nmoles/kg, about 0.1 nmoles/kg to about 10 nmoles/kg, or about 0.1 nmoles/kg to about 1 nmoles/kg of body weight of the subject. In other embodiments, the dose may be about 0.3 nmoles/kg to about 1000 nmoles/kg, about 0.3 nmoles/kg to about 900 nmoles/kg, about 0.3 nmoles/kg to about 850 nmoles/kg, about 0.3 nmoles/kg to about 800 nmoles/kg, about 0.3 nmoles/kg to about 700 nmoles/kg, about 0.3 nmoles/kg to about 600 nmoles/kg, about 0.3 nmoles/kg to about 500 nmoles/kg, about 0.3 nmoles/kg to about 400 nmoles/kg, about 0.3 nmoles/kg to about 300 nmoles/kg, about 0.3 nmoles/kg to about 200 nmoles/kg, about 0.3 nmoles/kg to about 100 nmoles/kg, about 0.3 nmoles/kg to about 50 nmoles/kg, about 0.3 nmoles/kg to about 10 nmoles/kg, or about 0.3 nmoles/kg to about 1 nmoles/kg of body weight of the subject.

In various other embodiments, the dose of the compound, or the pharmaceutically acceptable salt thereof, can range from, for example, about 10 nmoles/kg to about 10,000 nmoles/kg, from about 10 nmoles/kg to about 5,000 nmoles/kg, from about 10 nmoles/kg to about 3,000 nmoles/kg, about 10 nmoles/kg to about 2,500 nmoles/kg, about 10 nmoles/kg to about 2,000 nmoles/kg, about 10 nmoles/kg to about 1,000 nmoles/kg, about 10 nmoles/kg to about 900 nmoles/kg, about 10 nmoles/kg to about 800 nmoles/kg, about 10 nmoles/kg to about 700 nmoles/kg, about 10 nmoles/kg to about 600 nmoles/kg, about 10 nmoles/kg to about 500 nmoles/kg, about 10 nmoles/kg to about 400 nmoles/kg, about 10 nmoles/kg to about 300 nmoles/kg, about 10 nmoles/kg to about 200 nmoles/kg, about 10 nmoles/kg to about 150 nmoles/kg, about 10 nmoles/kg to about 100 nmoles/kg, about 10 nmoles/kg to about 90 nmoles/kg, about 10 nmoles/kg to about 80 nmoles/kg, about 10 nmoles/kg to about 70 nmoles/kg, about 10 nmoles/kg to about 60 nmoles/kg, about 10 nmoles/kg to about 50 nmoles/kg, about 10 nmoles/kg to about 40 nmoles/kg, about 10 nmoles/kg to about 30 nmoles/kg, about 10 nmoles/kg to about 20 nmoles/kg, about 200 nmoles/kg to about 900 nmoles/kg, about 200 nmoles/kg to about 800 nmoles/kg, about 200 nmoles/kg to about 700 nmoles/kg, about 200 nmoles/kg to about 600 nmoles/kg, about 200 nmoles/kg to about 500 nmoles/kg, about 250 nmoles/kg to about 600 nmoles/kg, about 300 nmoles/kg to about 600 nmoles/kg, about 300 nmoles/kg to about 500 nmoles/kg, or about 400 nmoles/kg to about 600 nmoles/kg.

In various other embodiments, the dose of the compound, or the pharmaceutically acceptable salt thereof, may range from, for example, about 1 nmoles/kg to about 10,000 nmoles/kg, from about 1 nmoles/kg to about 5,000 nmoles/kg, from about 1 nmoles/kg to about 3,000 nmoles/kg, about 1 nmoles/kg to about 2,500 nmoles/kg, about 1 nmoles/kg to about 2,000 nmoles/kg, about 1 nmoles/kg to about 1,000 nmoles/kg, about 1 nmoles/kg to about 900 nmoles/kg, about 1 nmoles/kg to about 800 nmoles/kg, about 1 nmoles/kg to about 700 nmoles/kg, about 1 nmoles/kg to about 600 nmoles/kg, about 1 nmoles/kg to about 500 nmoles/kg, about 1 nmoles/kg to about 400 nmoles/kg, about 1 nmoles/kg to about 300 nmoles/kg, about 1 nmoles/kg to about 200 nmoles/kg, about 1 nmoles/kg to about 150 nmoles/kg, about 1 nmoles/kg to about 100 nmoles/kg, about 1 nmoles/kg to about 90 nmoles/kg, about 1 nmoles/kg to about 80 nmoles/kg, about 1 nmoles/kg to about 70 nmoles/kg, about 1 nmoles/kg to about 60 nmoles/kg, about 1 nmoles/kg to about 50 nmoles/kg, about 1 nmoles/kg to about 40 nmoles/kg, about 1 nmoles/kg to about 30 nmoles/kg, or about 1 nmoles/kg to about 20 nmoles/kg.

In another embodiment, from about 20 μg/kg body weight to about 3 mg/kg body weight of the compound, or the pharmaceutically acceptable salt thereof, can be administered to the subject. In another aspect, amounts can be from about 0.2 mg/kg body weight to about 0.4 mg/kg body weight or can be about 50 μg/kg body weight.

Unless otherwise specified, in all the dosage embodiments set forth herein, “kg” is kilograms of body weight of the subject.

A single dose or multiple doses of the compound, or the pharmaceutically acceptable salt thereof, can be administered to the subject.

Any applicable dosing schedule known in the art can be used for administration of the compound, the pharmaceutically acceptable salt thereof, or a pharmaceutical composition comprising same. For example, once per day dosing (a.k.a. qd), twice per day dosing (a.k.a. bid), three times per day dosing (a.k.a. tid), twice per week dosing (a.k.a. BIW), three times per week dosing (a.k.a. TIW), once weekly dosing, and the like, can be used. In one aspect, the dosing schedule selected can take into consideration the concentration of the compounds/compositions being administered (including, for example, the number of CAR-T cells administered).

“Cancer” has its plain and ordinary meaning when read in light of the specification and can include, but is not limited to, a group of diseases involving abnormal cell growth with the potential to invade or spread to other parts of the body. Numerous types of cancers can be treated using the compositions, compounds, and methods described herein including, without limitation, a carcinoma, a sarcoma, an osteosarcoma, a lymphoma, a melanoma, a mesothelioma, a nasopharyngeal carcinoma, a leukemia, an adenocarcinoma, and a myeloma. Other, and perhaps more specific, examples of cancers that can be treated in accordance with the methods and/or using the compounds and compositions hereof include, but are not limited to, lung cancer (including, without limitation, non-small cell lung cancer), bone cancer (including, without limitation, osteosarcoma), pancreatic cancer, skin cancer (including, without limitation, cutaneous melanoma), cancer of the head, cancer of the neck, intraocular melanoma, uterine cancer, ovarian cancer, endometrial cancer, rectal cancer, stomach cancer, colon cancer, breast cancer, triple negative breast cancer, carcinoma of the fallopian tubes, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, prostate cancer, leukemia (including, without limitation, chronic leukemia, acute leukemia, acute myelocytic leukemia, a lymphocytic lymphoma, myeloid leukemia, myelomonocytic leukemia, and hairy cell leukemia), pleural mesothelioma, cancer of the bladder, Burkitt's lymphoma, cancer of the ureter, cancer of the kidney (including, without limitation, renal cell carcinoma), carcinoma of the renal pelvis, a neoplasm of the central nervous system (CNS), primary CNS lymphoma, a spinal axis tumor, a brain stem glioma, a pituitary adenoma, and an adenocarcinoma of the gastroesophageal junction.

In some aspects of these embodiments, the cancer is a folate receptor-expressing cancer, for example and without limitation, a folate receptor α-expressing cancer. In other embodiments, the cancer is a folate receptor β-expressing cancer. In some aspects of these embodiments, the cancer is an endometrial cancer, a non-small cell lung cancer, an ovarian cancer, or a triple-negative breast cancer.

The cancer being treated can be a tumor. In another embodiment, the cancer can be malignant. In another embodiment, the cancer is acute myelocytic leukemia such as, for example, an acute myelocytic leukemia where the cancer expresses folate receptor-β.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

In the above description, numerous specific details are set forth to provide a thorough understanding of the present disclosure. Particular examples may be implemented without some or all of these specific details and it is to be understood that this disclosure is not limited to particular biological systems, particular cancers, or particular organs or tissues, which can, of course, vary but remain applicable in view of the data provided herein.

Additionally, various techniques and mechanisms of the present disclosure sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

Further, will be understood that the disclosure is presented in this manner merely for explanatory purposes and the principles and embodiments described herein may be applied to compounds and/or composition components that have configurations other than as specifically described herein. Indeed, it is expressly contemplated that the components of the composition and compounds of the present disclosure may be tailored in furtherance of the desired application thereof.

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein. Additionally, as used in this specification and the appended claims, the singular forms “a”, “an” and “the” include plural referents unless the content clearly dictates otherwise. Thus, for example, where a compound/composition is substituted with “an” alkyl or aryl, the compound/composition is optionally substituted with at least one alkyl and/or at least one aryl.

When ranges are used herein for physical properties, such as molecular weight, or chemical properties, such as chemical formulae, all combinations and sub-combinations of ranges and specific embodiments therein are intended to be included.

Additionally, the term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +/−5% to 15% of the recited value) provided that one of ordinary skill in the art would consider equivalent to the recited value (e.g., having the same function or result). The term “comprising” (and related terms such as “comprise” or “comprises” or “having” or “including”) is not intended to exclude that in other certain embodiments, for example, an embodiment of any compound, composition of matter, composition, method, or process, or the like, described herein, may “consist of” or “consist essentially of” the described features. The term “substantially” can allow for a degree of variability in a value or range, for example, within 90%, within 95%, or within 99% of a stated value or of a stated limit of a range.

Where a method of therapy comprises administering more than one treatment, compound, or composition to a subject, it will be understood that the order, timing, number, concentration, and volume of the administration is limited only by the medical requirements and limitations of the treatment (i.e., two treatments can be administered to the subject, e.g., simultaneously, consecutively, sequentially, alternatively, or according to any other regimen).

Additionally, in describing representative embodiments, the disclosure may have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

It is therefore intended that this description and the appended claims will encompass, all modifications and changes apparent to those of ordinary skill in the art based on this disclosure.

### EXAMPLES

The following examples serve to illustrate the present disclosure. The examples are not intended to limit the scope of the claimed invention in any way.

### Example 1

### Cancer Cell Lines and Generation of Human Anti-Fluorescein CAR T-Cells

MDA-MB-231 and KB cells were obtained from the American Type Culture Collection (ATCC). Folic acid-free RPMI 1640 (Gibco; Thermo Fisher Scientific, Waltham, MA) containing 10% heat-inactivated fetal bovine serum (FBS) and 1% penicillin-streptomycin was used to culture both cell lines. To obtain stable mCherry-expressing MDA-MB-231 cells, MDA-MB-231 cells were first transduced with a lentiviral vector (pLv-NLS-mCherry-puro; Vector Builder, Chicago, IL) and then selected for positive clones in puromycin-containing media.

For generation of human anti-fluorescein chimeric antigen receptor (CAR) T-cells, peripheral blood mononuclear cells (PBMC) were isolated following informed consent from fresh human peripheral blood samples by Ficoll (GE Healthcare Life Sciences, Piscataway, NJ) density gradient centrifugation. CD3⁺ T-cells were then collected and enriched using an EasySep Human T-Cell Isolation Kit (STEMCELL Technologies, Vancouver, Canada), and human anti-fluorescein CAR T-cells were generated pursuant to known lentiviral vector protocols. All cells were maintained in 5% CO₂ at 37° C. and were regularly tested for contamination with Mycoplasma.

### Example 2

### Analysis of Expression of TLR7 in Human T-Cells

To determine whether Toll-like receptor 7 (TLR7) might be expressed in primary human T-cells, freshly isolated CD3⁺ T-cells were fixed and permeabilized according to the manufacturer's instructions (Intracellular Flow Cytometry Staining Protocol, BioLegend, San Diego, CA) and stained with Alexa Fluor 488-anti-TLR7 (IC5875G, R&D Systems, Minneapolis, MN). Cells were then washed 2 times with Intracellular Staining Permeabilization Wash Buffer (BioLegend, San Diego, CA) and resuspended in 1×phosphate-buffered saline (PBS) prior to analysis by flow cytometry.

### Example 3

Analysis of Fluorescein-NIR Dye Binding to Anti-Fluorescein CAR T-cells In Vitro and In Vivo

Anti-fluorescein CAR T-cells (along with MDA-MB-231 or KB cells as negative controls) were incubated with fluorescein-near infrared (NIR) dye (10 nM) for 1 hour at room temperature prior to incubation in the presence or absence of 1,000-fold excess sodium fluorescein (10 μM). Cells were then washed three times with PBS, and NIR dye fluorescence was measured by flow cytometry.

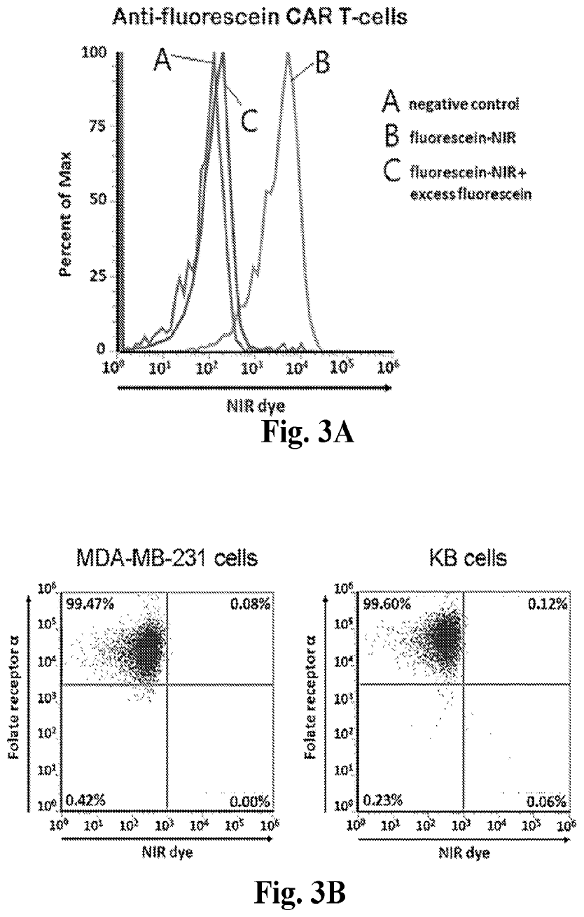

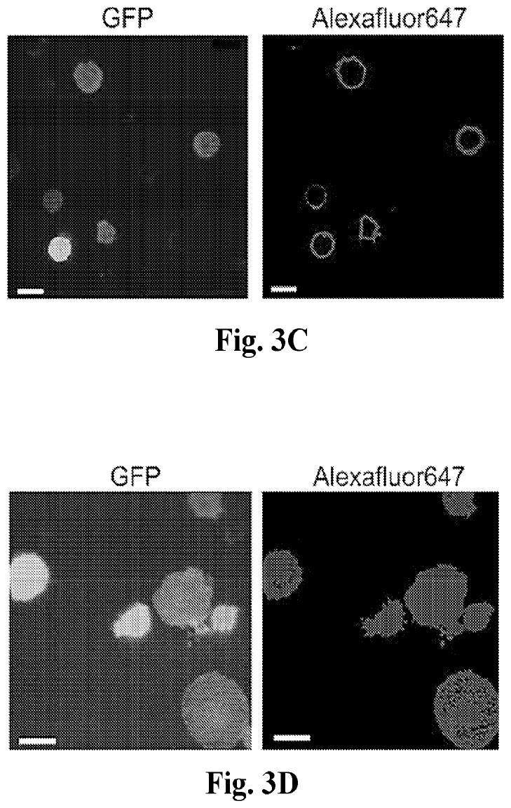

Flow cytometry of anti-fluorescein CAR T-cells in the absence (labeled A in FIG. 3A) or presence (labeled B in FIG. 3A) of the fluorescein-NIR dye conjugate (10 nM) or in the presence of both fluorescein-NIR dye conjugate plus 1,000-fold excess fluorescein (labeled C in FIG. 3A) demonstrated that the fluorescein-NIR dye conjugate binds to anti-fluorescein CAR T-cells in a manner that can be quantitatively blocked by addition of 1,000-fold excess fluorescein.

In addition, NSG mice were implanted with 1 million KB cells in one flank and infused with anti-fluorescein CAR T-cells (8×10⁶ cells that contained ˜50% anti-fluorescein CAR T-cells) when KB tumor volumes reached ˜50 mm³. Fluorescein-folate (500 nmol/kg) was injected 4 hours and 24 hours later and weekly thereafter. Fifteen days after CAR T-cell infusion, the mice were tail vein-injected with 500 nmol/kg fluorescein-NIR dye. Four hours later tumors were dissociated and analyzed by flow cytometry for uptake of fluorescein-NIR dye. CD3+ T-cells were detected with anti-human CD3 on the APC-Cy7 channel, and green fluorescent protein (GFP)-transfected anti-fluorescein CAR T-cells were detected using the GFP channel.

As shown in FIG. 3B, CAR-negative cells (e.g., MDA-MD-231 cells and KB cells), do not express binding sites for fluorescein-dye conjugates (10 nM). In contrast, confocal microscopic evaluation shows that fluorescein-Alexafluor 647 conjugate is endocytosed by CAR T-cells, which have an anti-fluorescein cell-surface receptor (FIG. 3C shows Alexafluor 647 endocytosis after incubation for 1 hour at 4° C., whereas FIG. 3D shows Alexfluor 647 endocytosis after subsequent transfer of the CAR T-cells to 37° C. for 4 hours). Bar=10 μm.

### Example 4

### Stimulation of CAR T-Cell Exhaustion and Reversal of Exhaustion In Vitro

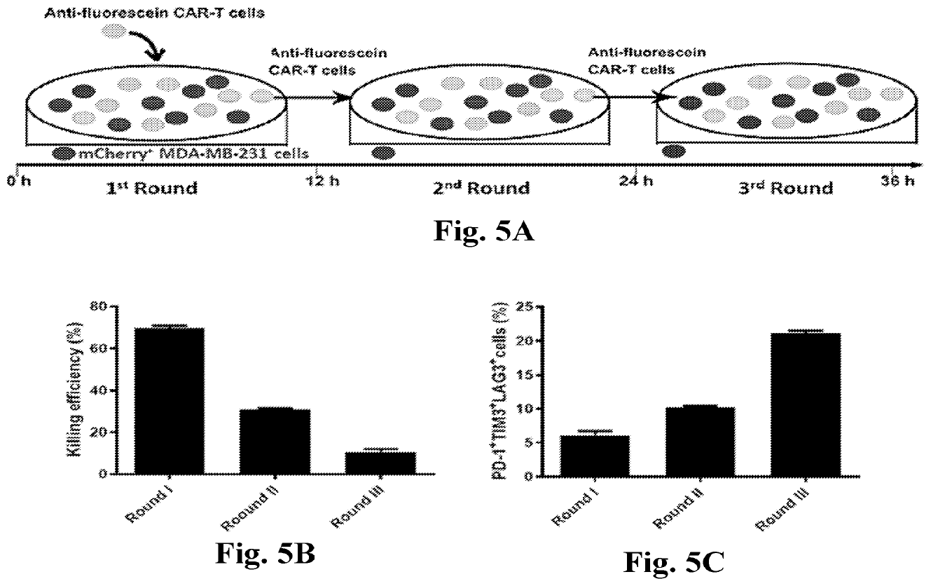

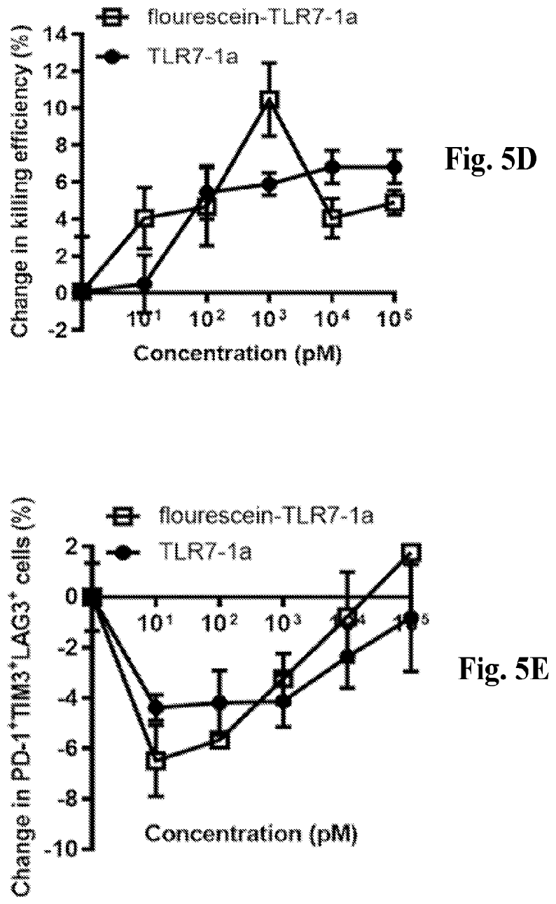

To induce CAR T-cell exhaustion, anti-fluorescein CAR T-cells (10⁴/well) were added to folate receptor-expressing mCherry+ MDA-MB-231 cells (10⁴/well) in folic acid-free RPMI 1640 medium, after which CAR T-cell mediated killing of the mCherry+ MDA-MB-231 cells was initiated by addition of 10 nM fluorescein-folate. Then, every 12 hours thereafter, the anti-fluorescein CAR-T cells were transferred into a fresh flask of mCherry+ MDA-MB-231 cells (10⁴/well) to assure continuous exposure to tumor antigen as shown in FIG. 5A. A fraction of the anti-fluorescein CAR-T cells were harvested after 12 hours (1^{st} Round), 24 hours (2^{nd} Round), and 36 hours (3^{rd} Round) to analyze expression of exhaustion markers PD-1, TIM3, and LAG3 by flow cytometry. CAR T-cells were considered exhausted when they simultaneously expressed PD-1, TIM3 and LAG3. As shown in FIG. 5C, the T-cell exhaustion markers PD-1, TIM3, and LAG3, increased. The number of live mCherry+ (MDA-MB-231) cells was counted by Incucyte S3 every 4 hours and was used to calculate the cancer cell-killing efficiency. As shown in FIG. 5B, the ability of the anti-fluorescein CAR T-cells to kill MDA-MB-231 cells decreased.

For assessment of exhausted CAR T rejuvenation, at the start of the third round of exhaustion conditioning, the combined CAR T-cell plus MDA-MB-231 cell culture was incubated overnight with the desired rejuvenating compounds at concentrations ranging from 0.01-100 nM. CAR T-cells were then evaluated for rejuvenation by quantitating their cancer cell-killing efficiency and analyzing their expression of PD-1, TIM3 and LAG3.

Rejuvenation of the exhausted anti-fluorescein CAR T-cells was demonstrated by incubation with either targeted or non-targeted TLR7-1a. As shown in FIG. 5E, the T-cell exhaustion markers programmed cell death protein 1 (PD-1V), T-cell immunoglobulin and mucin-domain containing-3 (TIM3⁺), and lymphocyte activating 3 (LAG3⁺)) decreased. Further, as shown in FIG. 5D, the ability of the anti-fluorescein CAR T-cells to kill MDA-MB-231 cells increased. Data shown represents the change in the markers over baseline levels (i.e., vehicle (dimethyl sulfoxide (DMSO)-treated)). Bar graphs present mean±SD, n=3.

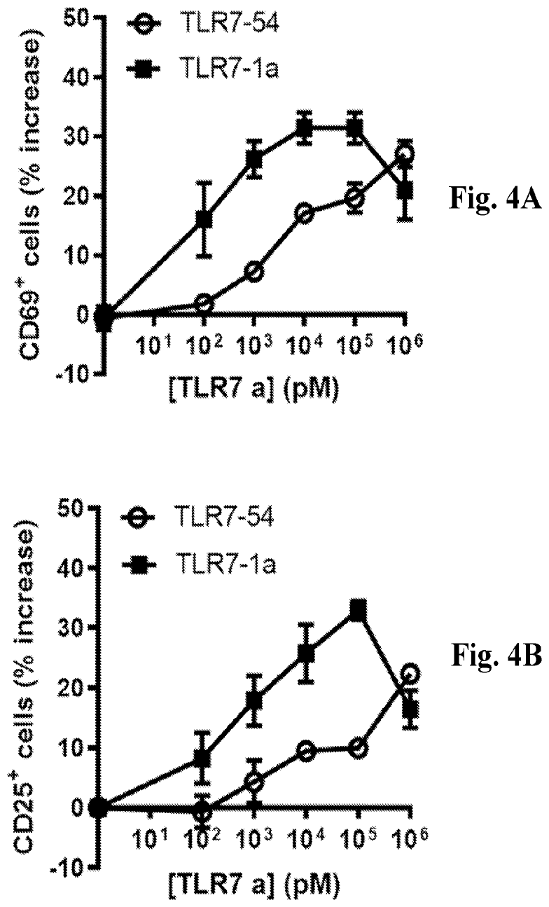

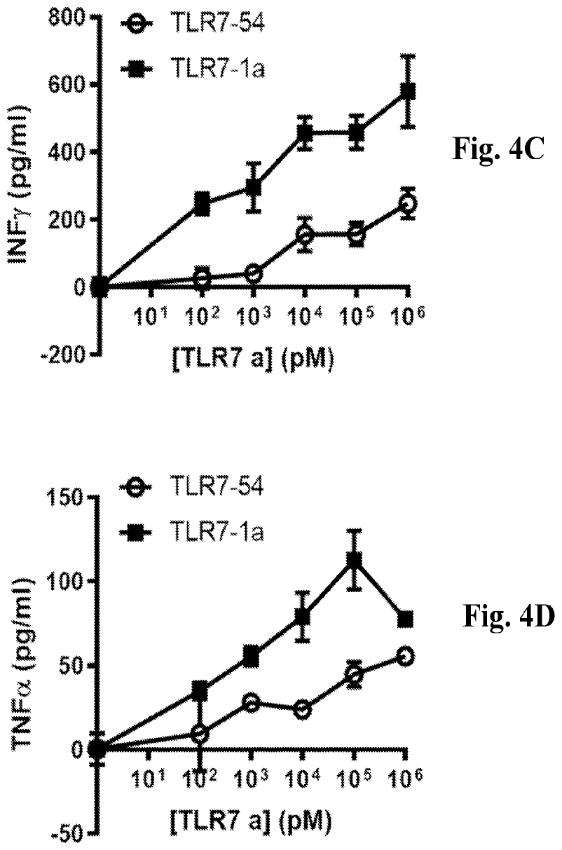

Human CD3+ T-cells were activated upon administration of different concentrations of either TLR7-54 or TLR7-1a agonist as shown in FIG. 4. FIG. 4A shows the percent increase in CD69+ cells after isolated human peripheral blood CD3+ T-cells were stimulated with anti-CD3+ monoclonal antibody (mAb) in the absence or presence of increasing concentrations of TLR-54 or TLR7-1a as measured by flow cytometry. FIG. 4B shows the percent increase in CD25+ cells after isolated human peripheral blood CD3+ T-cells were stimulated with anti-CD3+ mAb in the absence or presence of increasing concentrations of TLR-54 or TLR7-1a as measured by flow cytometry. FIG. 4C shows the level of interferon gamma (INF-γ) in the 24-hour cell-free supernatants of the stimulated T-cells as measured by enzyme-linked immunosorbent assay (ELISA). FIG. 4D shows the level of tumor necrosis factor alpha (TNF-α) in the 24-hour cell-free supernatants of the stimulated T-cells as measured by ELISA. Data shown supports an increase in these parameters over baseline levels (i.e., vehicle (DMSO-treated)). Bar graphs represent mean±SD, n=3.

### Example 5

### Rejuvenation of Anti-Fluorescein CAR T-Cells In Vivo Following Intravenous Injection of Fluorescein-TLR7-1a Conjugate

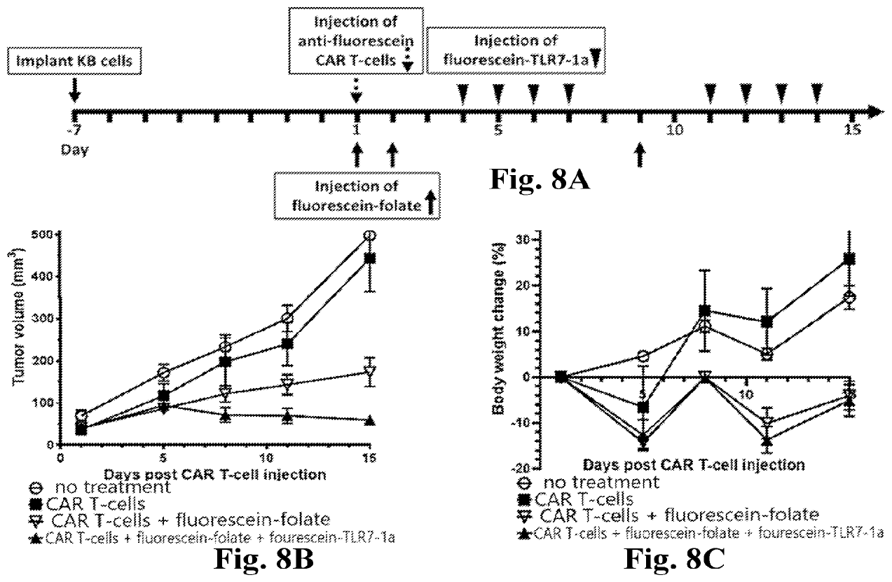

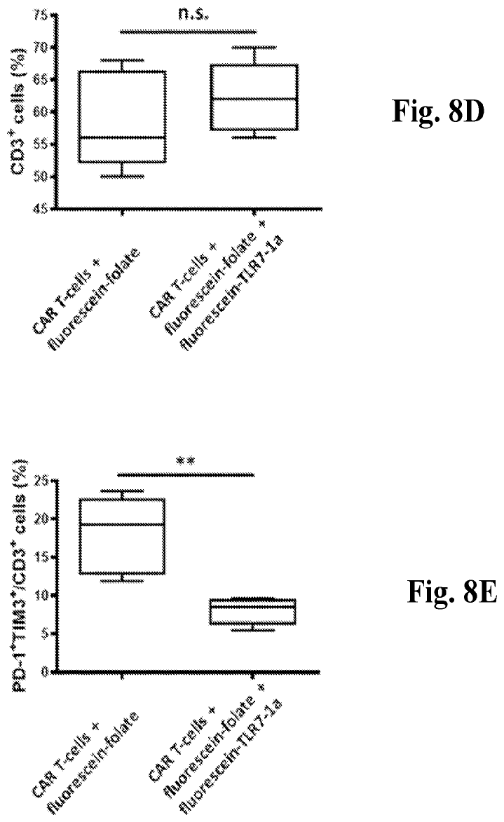

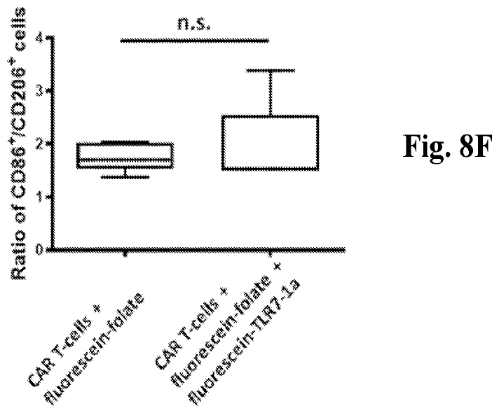

Mice were injected subcutaneously on day −7 with 10⁶ KB cells and then divided into four groups, with the treatment groups infused on day 1 with 8×10⁶ anti-fluorescein CAR T-cells. The control group did not receive CAR T-cells. At 6 hours, 24 hours, and 9 days later, two of the treatment group mice were intravenously injected with fluorescein-folate to induce engagement of CAR T-cells with folate receptor-positive KB cancer cells. Thereafter, the treatment group mice were divided into two cohorts, and on days 4-7 and days 11-14, the mice of the second cohort of the treatment group were intravenously injected with fluorescein-TLR7-1a. The timeline for the in vivo study is shown in FIG. 8A.

Tumor volumes (FIG. 8B) and animal body weight changes (FIG. 8C) were measured every 3 days. The cohort treated with CAR T+fluorescein-folate+fluorescein-TLR7-1a exhibited retarded tumor growth as compared with all other groups (both controls and the other cohort of the treatment group). The cohort treated with CAR T+fluorescein-folate+fluorescein-TLR7-1a showed negligible percent body weight change as compared with the cohort treated with CAR T+fluorescein-folate.

Tumors were resected and dissociated into component cells on day 16, and human CD3+ T-cells were determined as a percentage of all tumor cells (FIG. 8D). PD-1+ TIM3+ cells as a percentage of all human CD3+ T-cells also was determined (FIG. 8E), as was the ratio of mouse CD86+ to CD206+ cells also expressing myeloid markers F4/80 and CD11b in the anti-fluorescein CAR T-cell treatment groups (FIG. 8F). Our targeted delivery of TLR7-1a has successfully rejuvenated the exhausted CAR T cells without affecting the myeloid cells. All data were plotted as mean±SEM. Data shown are representative of at least two independent experiments. Data were analyzed by two-way analysis of variance (ANOVA) (**p<0.01; n.s.=not significant).

### Example 6

### Comparison of CAR T-Cell Eradication of Solid KB and MDA-MB-231 Cell Tumors in the Presence and Absence of Fluorescein-TLR7-1a Conjugate

Eight- to 10-weeks old NSG mice (Jackson Lab strain No. 005557; The Jackson Laboratory, Bar Harbor, ME) were transferred upon arrival to folic acid-deficient diet (TD.95247, Envigo, Indianapolis, IN) to lower their serum folic acid concentrations to levels similar to those in humans and mice in the wild. One week later, the mice were implanted subcutaneously with ˜10⁶ KB cells and 4 million MDA-MB-231 cells on separate flanks of the same mice. Tumors were allowed to grow about two weeks until KB tumors reach 80 mm³ and MDA-MD-231 tumors reached ˜160 mm³ (i.e., to accommodate their different growth rates). Mice were then injected intravenously with saline or 8×10⁶ anti-fluorescein CAR T-cells, followed by injections with 500 nmol/kg fluorescein-folate both 4 hours and 24 hours after CAR T-cell infusion.

Four days-post CAR T-cell infusion, the CAR T-cell treated mice were divided into a rejuvenation group (fluorescein-TLR7 treated) and a control group (saline treated). The fluorescein-TLR7 treated cohort received 500 nmol/kg of fluorescein-TLR7 4×/week, while the control group received an equal volume of saline on the same schedule. Tumor volume was concurrently measured with calipers using the formula (1×w²)/2 (“l” being the largest length across the tumor and “w” being the dimension perpendicular to the longest transcept).

On day 18, mice were sacrificed, and tumor fragments dissociated using a human tumor dissociation kit (130-095-929, MeMiltenyi Biotec, Bergisch Gladbach, Germany). The resulting single-cell suspensions were stained for human CD3 (anti-CD3-APC-Cy7, BioLegend, Sand Diego, CA), PD-1 (anti-PD-1-PE, BioLegend, Sand Diego, CA), TIM3 (anti-TIM3-PE-Cy7, BioLegend, Sand Diego, CA), mouse CD11b (anti-CD11b-PE, BioLegend, Sand Diego, CA), F4/80 (anti-F4/80-APC-Cy7, BioLegend, Sand Diego, CA), M2 macrophage marker CD206 (anti-CD206-APC, BioLegend, Sand Diego, CA), and M1 macrophage marker CD86 (anti-CD86-PE-Cy7, BioLegend, Sand Diego, CA). All the samples were then analyzed by flow cytometry.

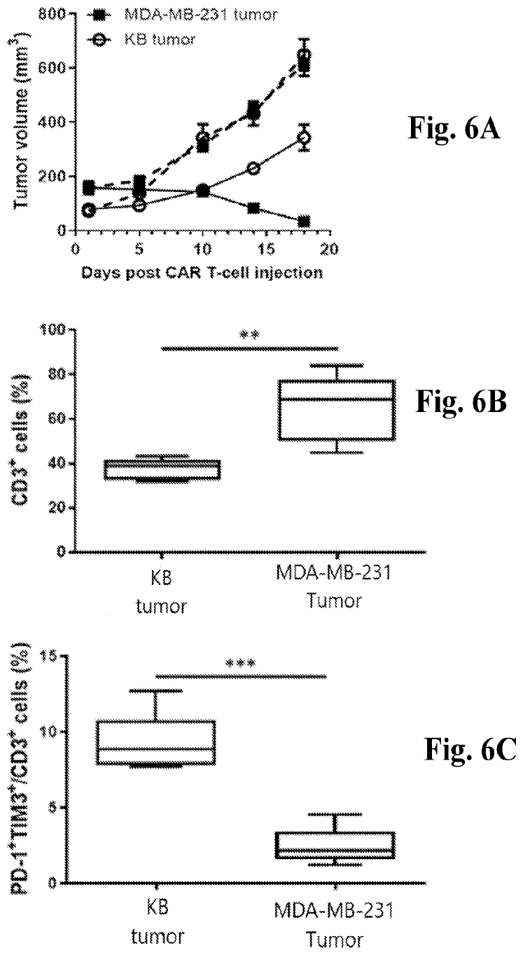

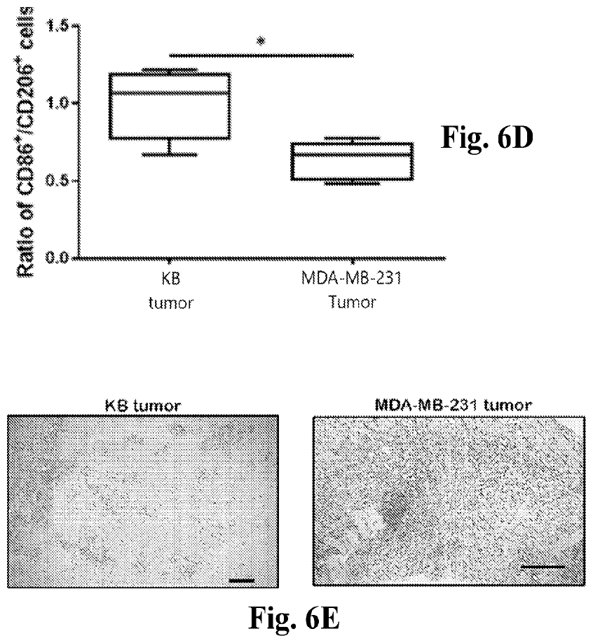

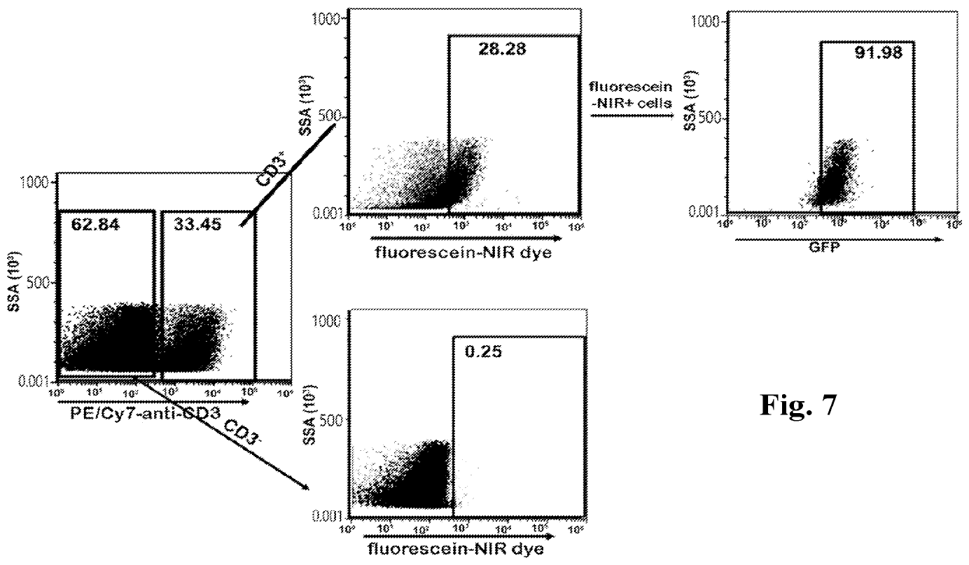

FIG. 6 shows the effect of anti-fluorescein CAR T-cell therapy on the growth and immunologic properties of MDA-MB-231 and KB tumors. FIG. 6A shows the tumor volume in cohorts either left untreated (dashed lines) or treated with both anti-fluorescein CAR T-cells plus fluorescein-folate bispecific adapter (solid lines). FIG. 6B shows the percentage of CD3+ T-cells (as a percentage of total cells in tumor) on day 18, when tumors were resected and both cancer and stromal cells were released using a tumor dissociation kit prior to analysis by flow cytometry. FIG. 6C shows the exhaustion markers PD-1+ and TIM3+ (as a percentage of all human CD3+ T-cells) on day 18, when tumors were resected and both cancer and stromal cells were released using a tumor dissociation kit prior to analysis by flow cytometry. FIG. 6D shows the ratio of CD86+

F4/80+ CD11b+ to CD206+ F4/80+ CD11b+ myeloid cells on day 18, when tumors were resected and both cancer and stromal cells were released using a tumor dissociation kit prior to analysis by flow cytometry. n=5 mice/group. All data were analyzed by two-way ANOVA and plotted as mean±SEM (*p<0.05, **p<0.01, ***p<0.001). Data shown are representative of two independent experiments. FIG. 6E shows representative images showing human CD3+ T-cell infiltrations into MDA-MB-231 and KB cell solid tumors. Bar=200 μm.

### Chemistry Examples

### Example A

### Synthesis of FITC-TLR7-1a

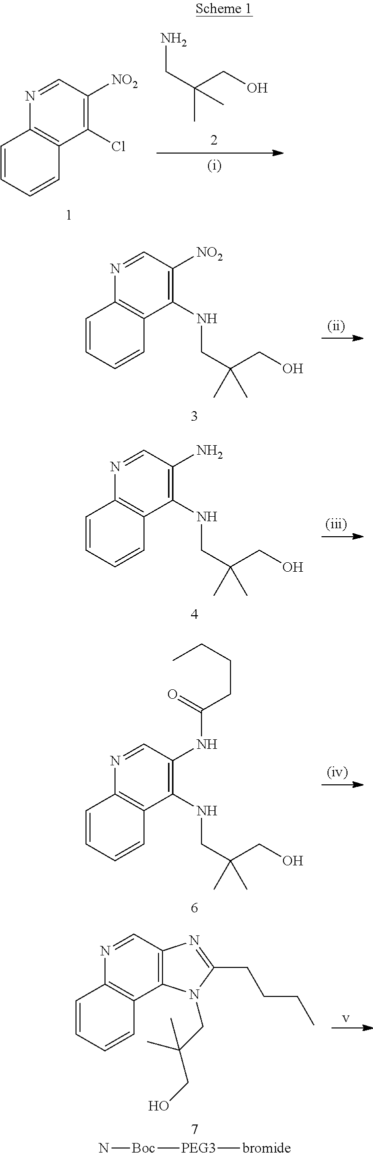

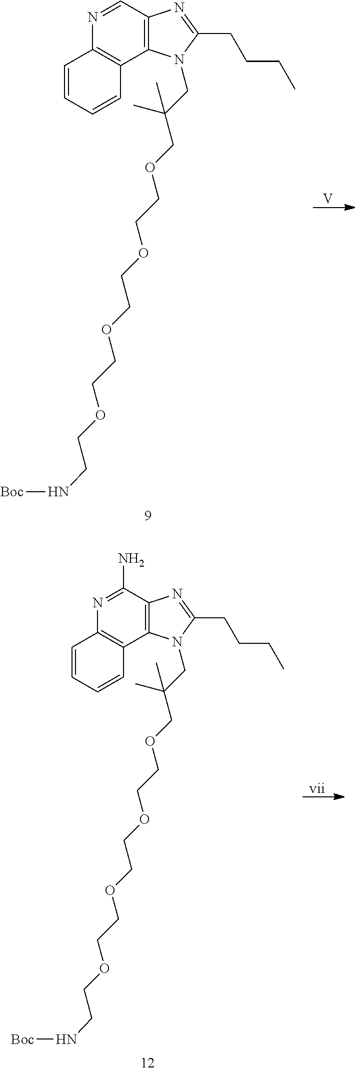

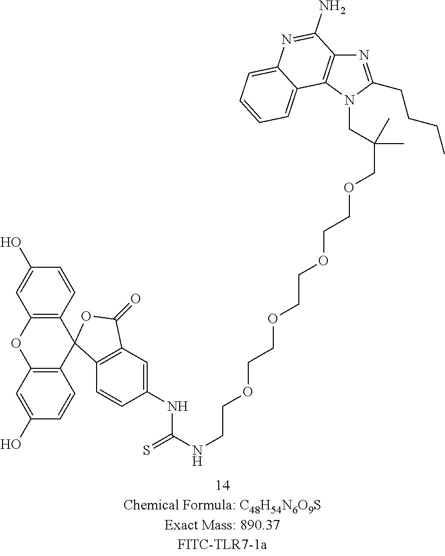 [SMILES: CCCCc1nc2c(N)nc3ccccc3c2n1CC(C)(C)COCCOCCOCCOCCNC(=S)Nc1ccc2c(c1)C(=O)OC21c2ccc(O)cc2Oc2cc(O)ccc21]

3-amino-2,2-dimethylpropan-1-ol (2, 1.5 equiv) and triethyl amine (2 equiv) were added to a stirred solution of 4-chloro-3-nitroquinoline (1, 1.2 equiv) in N,N-dimethyl formamide (DMF) (10 mL) ((i) of Scheme 1). The reaction mixture was heated at 70° C. for 60 minutes and monitored through liquid chromatography-mass spectrometry (LCMS). It was then cooled down, diluted with water, and stirred for another 15 minutes. The precipitated solid was filtered and washed with water. The solid was dried under vacuum to get material 3 as yellow solid. Yield—80%.

Thereafter, 2,2-dimethyl-3-(3-nitroquinolin-4-ylamino)propan-1-ol (1 g) was dissolved in methanol (15 mL) and reduced over Pd/C (100 mg, 10 mol %) as catalyst under hydrogen balloon condition for 4 hours ((ii) of Scheme 1). The solution was then filtered and evaporated under reduced pressure to get 3-(3-aminoquinolin-4-ylamino)-2,2-dimethylpropan-1-ol (4).

Triethylamine (2 equiv) and valeryl chloride (5, 1.5 equiv) were then added to a stirred solution of material 4 (1 g) in anhydrous tetrahydrofuran (THF) (10 mL) ((iii) of Scheme 1). The reaction mixture was then stirred for 4 hours, followed by removal of the solvent under reduced pressure. The crude residue was then dissolved in ethyl acetate (EtOAc), washed with water, brine and dried over sodium sulphate. The combined organic layer was evaporated to dryness under vacuum to obtain the intermediate amide compound 6. Overall yield—70%.

To a stirred solution of the amide compound 6 (1 g) in methanol (MeOH) (15 mL) was added an excess of calcium oxide (10 equiv), and the solution was heated at 110° C. for 96 hours ((iv) of Scheme 1). The solvent was then removed under vacuum, and the residue purified using flash column chromatography (MeOH/dichloromethane mobile phase) to obtain compound 7. Yield—60%.

NaH (2 equiv) and N-Boc-PEG3-bromide (8, 2 equiv) were then added to a stirred solution of the intermediate hydroxyl compound 7 (500 mg) in THF (5 mL) ((v) of Scheme 1). This was stirred under nitrogen atmosphere for about 5 hours, and then the solvent was evaporated to dryness using a rotary evaporator. It was quenched with water and diluted with DMSO. The crude reaction mixture was purified through high-performance liquid chromatography (HPLC) using ammonium acetate and acetonitrile as the mobile phase to get the product as a colorless liquid (9). Yield—60%.

To a stirred solution of 9 (100 mg) in anhydrous dichloromethane (1 mL) was added 3-chloroperoxybenzoic acid (10, 1.5 equiv), and the solution was refluxed at 45° C. for 30 minutes ((vi) of Scheme 1). When the starting material was completely consumed, the solvent was evaporated to dryness under vacuum. The residue was then re-dissolved in anhydrous dichloromethane (1 mL), followed by the addition of trichloroacetyl isocyanate (11, 2.0 equiv), and the reaction mixture was heated at 45° C. for 30 minutes ((vi) of Scheme 1). After the completion of the reaction, the solvent was removed under vacuum, and the residue was re-dissolved in anhydrous MeOH (1 mL), followed by the addition of 25% methanolic sodium methoxide solution (0.2 mL). This was then heated at 75° C. for an hour and cooled down ((vi) of Scheme 1). The solvent was removed under vacuum, and the residue was purified using column chromatography (MeOH/dichloromethane) to obtain the compound 12 as colorless liquid. Overall yield-40%.

To a stirred solution of the intermediate compound 12 (100 mg) in dichloromethane (1 mL) was added trifluoroacetic acid (10 equiv) ((vii) of Scheme 1). This was stirred under nitrogen atmosphere for about 1 hour, and the solvents were evaporated to dryness using rotary evaporator. After the complete removal of residual acid, it was dissolved in DMSO (1 mL).

To this DMSO solution fluorescein isothiocyanate (FITC) (catalogue no-F7250; Sigma Aldrich, St. Louis, MO) (13, 1.2 equiv) and DIPEA (2 equiv) were added, and the solution was stirred for 10 minutes ((vii) of Scheme 1). The reaction was monitored through LCMS. After the starting materials were completely consumed, it was purified through HPLC using ammonium acetate and acetonitrile as the mobile phase to get the product (14) (FITC-TLR7-1a) as a yellow solid. yield—90%.

### Example B

### Synthesis of Fluorescein-Drug Conjugates

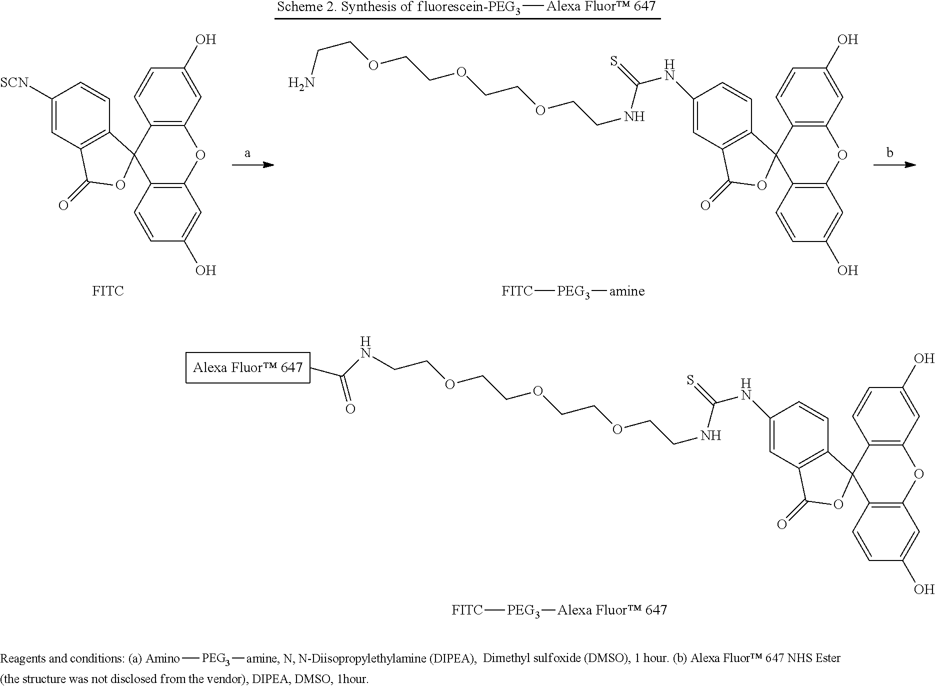

FITC was added dropwise to a solution of amino-PEG₃-amine (3 equiv) and DIPEA (5 equiv) in DMSO. The solution was stirred at room temperature for 1 hour. The resulting product was purified by preparative reverse-phase high-performance liquid chromatography (HPLC) (yield of 92%). To get FITC-PEG₃-Alexa Fluor 647, Alexa Fluor 647 NHS ester (Thermo Fisher Scientific, Waltham, MA). FITC-PEG₃-amine (2 equiv) and DIPEA (5 equiv) were dissolved in DMSO, and the solution was stirred for 1 hour. The product was then purified using HPLC to get the compound in 90% yield.

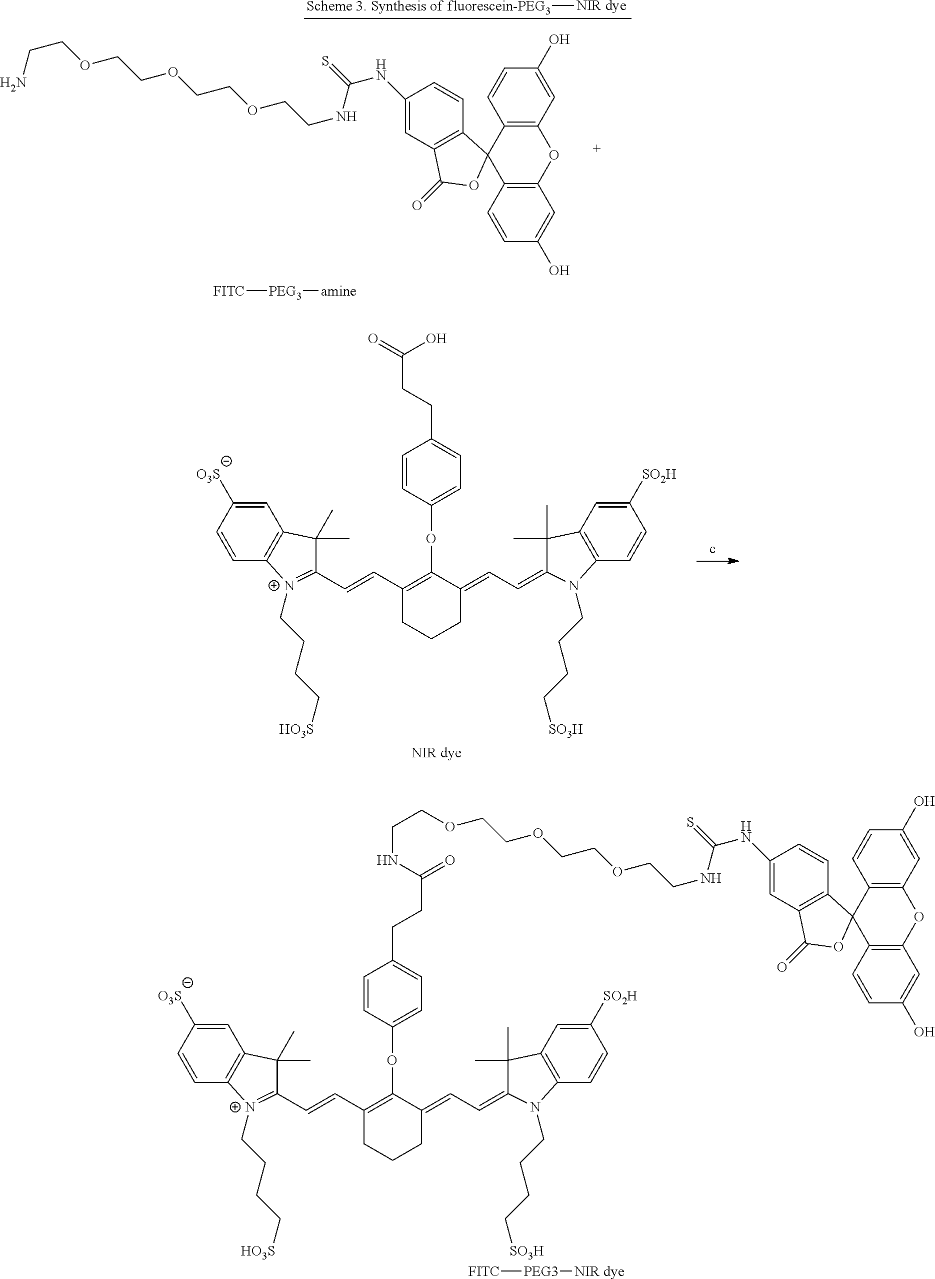

Reagents and conditions: (c) HATU, DIPEA, DMSO, 12 hours.

Near-infrared fluorescent (NIR) dye, HATU (1 equiv), and DIPEA (5 equiv) were dissolved in DMOS and stirred for 25 minutes, followed by addition of FITC-PEG₃-amine (1 equiv). The reaction was stirred at room temperature for 12 hours and the product was purified using HPLC (71%).

1. A method of rejuvenating exhausted chimeric antigen receptor (CAR) T-cells in a subject with a cancer being treated with:
(a) (i) a vector comprising a promoter operably linked to a nucleic acid sequence encoding a CAR or (ii) T-cells expressing the CAR, wherein the CAR binds a first targeting moiety, a second targeting moiety, or the first targeting moiety and the second targeting moiety, and
(b) a cancer-binding conjugate comprising a ligand that binds a cancer cell with specificity and conjugated with the first targeting moiety, wherein the ligand bound by the cancer and the first targeting moiety are optionally conjugated via a first linker, which method comprises:
administering to the subject a CAR T-cell-rejuvenating conjugate comprising an agonist of toll-like receptor 7, toll-like receptor 8, or toll-like receptor 7 and toll-like receptor 8 (agonist of TLR7/8) conjugated with either the first targeting moiety or the second targeting moiety, wherein the agonist and either of the first targeting moiety or the second targeting moiety are conjugated via a second linker,
wherein the CAR T-cell-rejuvenating conjugate has the structure: 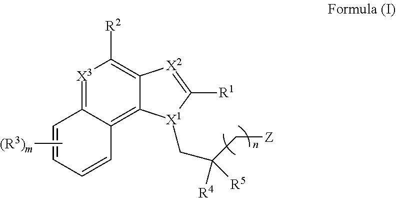
or a pharmaceutically acceptable salt thereof, wherein:
R¹ is an acyclic or cyclic alkyl, which is optionally substituted with one or more substituents independently selected from an alkyl, halo, heteroalkyl, alkoxy, and cycloalkyl;
R² is —NR^{2x}R^{2y}, hydrogen (H), —OR^{z}, —SO₂N(R^{z})₂, or N₃, wherein:
R^{2x} and R^{2y} are each independently H, —N(R^{z})₂, —CON(R^{z})₂, —C(R^{z})₂—N(R^{z})₂, —CS—N(R^{z})₂, or an alkyl that is optionally substituted with one or more substituents independently selected from oxo, halo, alkyl, heteroalkyl, alkoxy, and cycloalkyl;
each R^{z} is independently H or alkyl, which is optionally substituted; or
R^{2x} and R^{2y} are taken together to form a 3-10 membered mono- or bicyclic heterocycloalkyl, which is optionally substituted;
each R³ is independently H, halo, —N₃, —CN, —NO₂, alkyl, heteroalkyl, cycloalkyl, heterocycloalkyl, alkoxy, aryl, heteroaryl, heterocycloalkyl, amino, hydroxy, carbonyl, or thiol, wherein the alkyl, heteroalkyl, cycloalkyl, heterocycloalkyl, or alkoxy that is optionally substituted;
R⁴ and R⁵ are each independently alkyl, alkoxy, halo, or cycloalkyl, wherein the alkyl, alkoxy, or cycloalkyl that is optionally substituted;
X¹, X², and X³ are independently N or CR^{q}, wherein each R^{q} is independently H, halo, or an optionally substituted alkyl;
Z is G-L-, wherein L is the second linker and G the first a targeting moiety or the second targeting moiety;
n in Formula (I) is 1-6; and
m in Formula (I) is 0-4; or
wherein the CAR T-cell-rejuvenating conjugate has the structure: 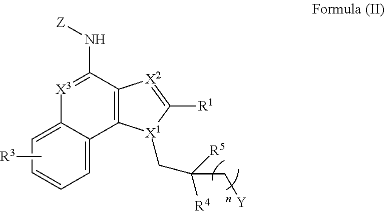
or is a pharmaceutically acceptable salt thereof, wherein:
R¹, R³, R⁴, and R⁵ are each independently H, alkyl, alkoxyl, alkenyl, alkynyl, alicyclic, aryl, biaryl, halo, heteroaryl, —COR^{2x}, 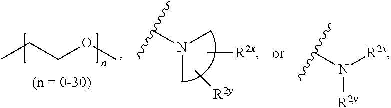
wherein each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl;
Z is G-L-, wherein L is the second linker and G is the first targeting moiety or the second targeting moiety;
X¹, X², and X³ are independently CR^{q} or N, wherein each R^{q} is independently H, halo, or optionally substituted alkyl;
Y is H, —OR^{z}, —NR^{2x}R^{2y}, —SR^{z}, —SOR^{z}, —SO₃R^{z}, —N₃, —COR^{z}, —COOR^{z}, —CON(R^{z})₂, —COSR^{z}, —SO₂N(R^{z})₂, or —CON(R^{z})₂, wherein:
R^{2x} and R^{2y} are each independently H, —N(R^{z})₂, —CON(R^{z})₂, —C(R^{z})₂—N(R^{z})₂, —CS—N(R^{z})₂, or optionally substituted alkyl;
each R^{z} is independently H or optionally substituted alkyl; or
R^{2x} and R^{2y} are taken together to form an optionally substituted heterocycloalkyl; and
n in Formula (II) is 0-30; or
wherein the CAR T-cell-rejuvenating conjugate has the structure: 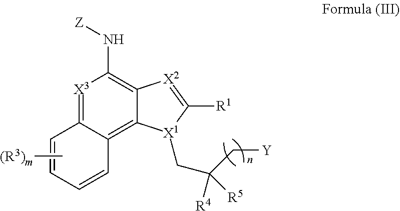
or is a pharmaceutically acceptable salt thereof, wherein:
R¹ is an acyclic or cyclic alkyl, which is optionally substituted with one or more substituents independently selected from halo, alkyl, heteroalkyl, alkoxy, and cycloalkyl;
Y is H, —OR^{z}, —NR^{2x}R^{2y}, —SR^{z}, —SOR^{z}, —SO₃R^{z}, —N₃, —COR^{z}, —COOR^{z}, —CONR^{z}₂, —COSR^{z}, —SO₂N(R^{z})₂, or —CON(R^{z})₂, wherein: R^{2x} and R^{2y} are each independently H, —N(R^{z})₂, —CON(R^{z})₂, —C(R^{z})₂—N(R^{z})₂, —CS—N(R^{z})₂, or an alkyl that is optionally substituted with one or more substituents independently selected from oxo, halo, alkyl, heteroalkyl, alkoxy, and cycloalkyl;
each R^{z} is independently H or an optionally substituted alkyl; or
R^{2x} and R^{2y} are taken together to form a 3-10 membered mono- or bicyclic heterocycloalkyl, which is optionally substituted;
each R³ is independently halo, —N₃, —CN, —NO₂, alkyl, heteroalkyl, cycloalkyl, heterocycloalkyl, alkoxy, aryl, heteroaryl, heterocycloalkyl, amino, hydroxy, carbonyl, or thiol, wherein the alkyl, alkoxy, heteroalkyl, cycloalkyl, or heterocycloalkyl that is optionally substituted;
R⁴ and R⁵ are each independently alkyl, alkoxy, halogen, or cycloalkyl, wherein the alkyl, alkoxy, or cycloalkyl and is optionally substituted;
X¹, X², and X³ are independently CR^{q} or N, wherein each R^{q} is independently H, halogen, or optionally substituted alkyl;
Z is L-G, wherein L is the second linker and G is the first targeting moiety or the second targeting moiety;
n in Formula (III) is 0-30; and
m in Formula (III) is 0-4.

2. The method of claim 1, wherein each of X¹, X², and X³ of Formula (I), Formula (II), or Formula (III) is N.

3. The method of claim 2, wherein the CAR T-cell-rejuvenating conjugate has the structure: 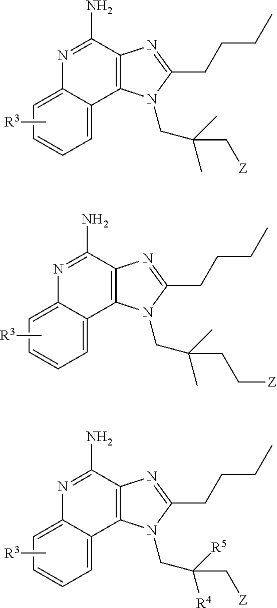
or is a pharmaceutically acceptable salt of any of the foregoing structures.

4. The method of claim 1, wherein the CAR T-cell-rejuvenating conjugate has the structure: 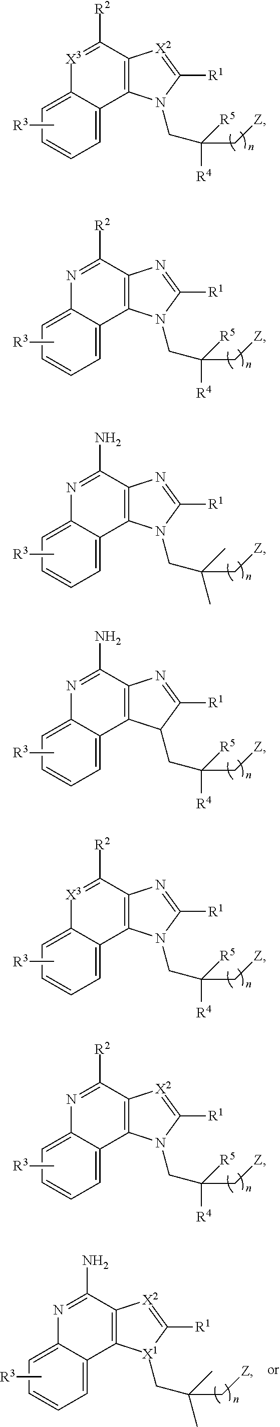 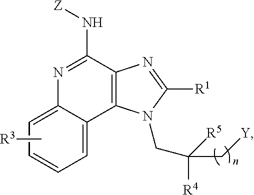
or is a pharmaceutically acceptable salt of any of the foregoing structures.

5. The method of claim 1, wherein the CAR T-cell-rejuvenating conjugate comprises an agonist of TLR7/8 and has the structure: 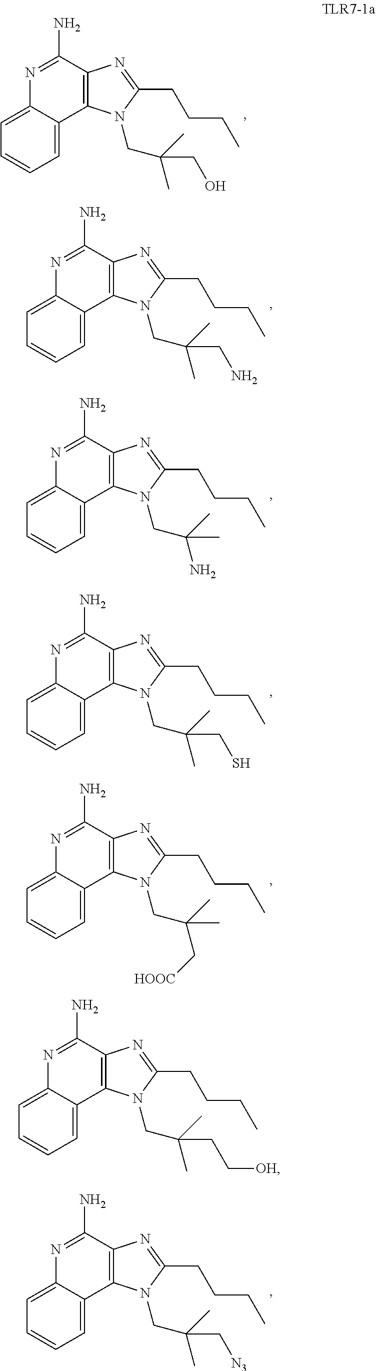 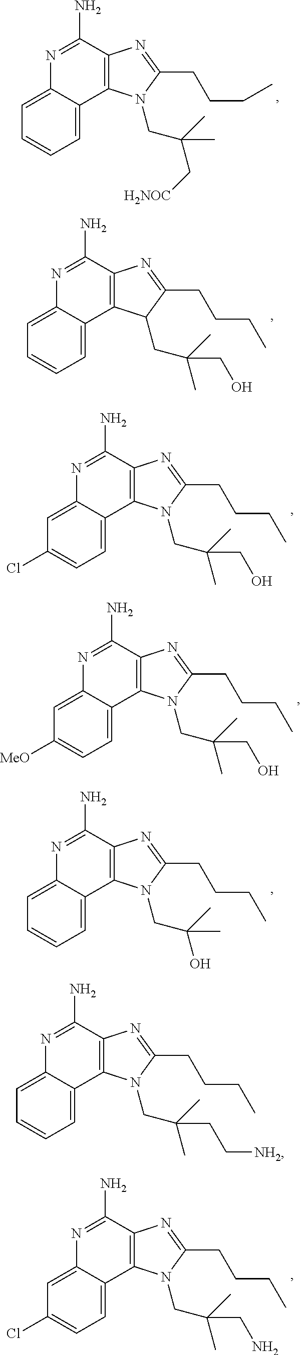 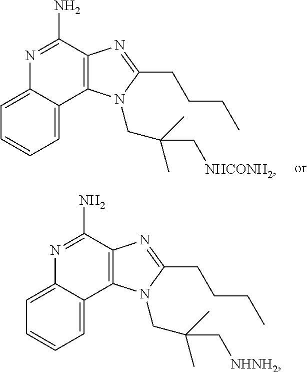
or is a pharmaceutically acceptable salt of any of the foregoing structures.

6. The method of claim 1, wherein the first targeting moiety, the second targeting moiety, or the first targeting moiety and the second targeting moiety is/are a group or comprise(s) a group with the structure:

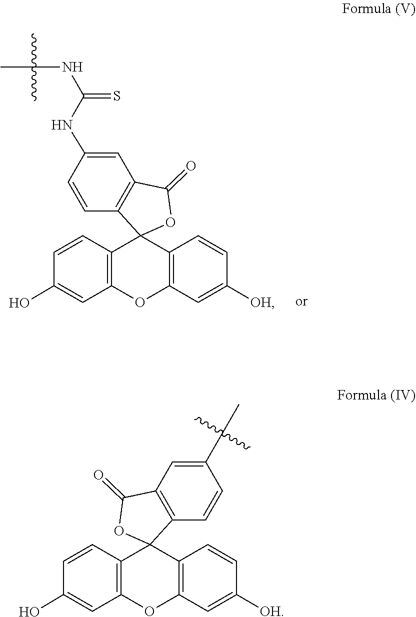 [SMILES: *NC(=S)Nc1ccc2c(c1)C(=O)OC21c2ccc(O)cc2Oc2cc(O)ccc21 ; "or" ; *c1ccc2c(c1)C(=O)OC21c2ccc(O)cc2Oc2cc(O)ccc21]

7. (canceled)

8. The method of claim 1, wherein the CAR T-cell-rejuvenating conjugate has the structure: 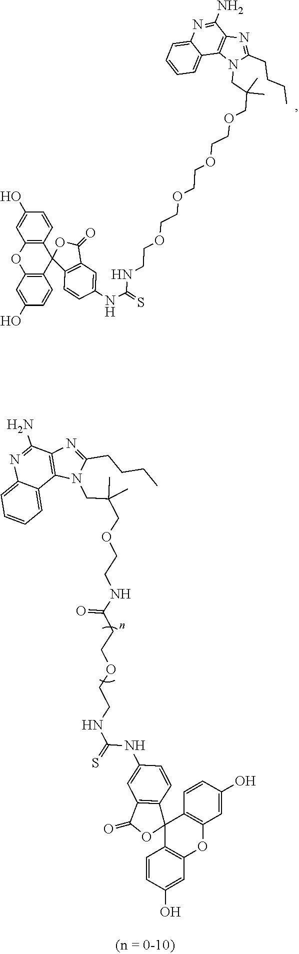 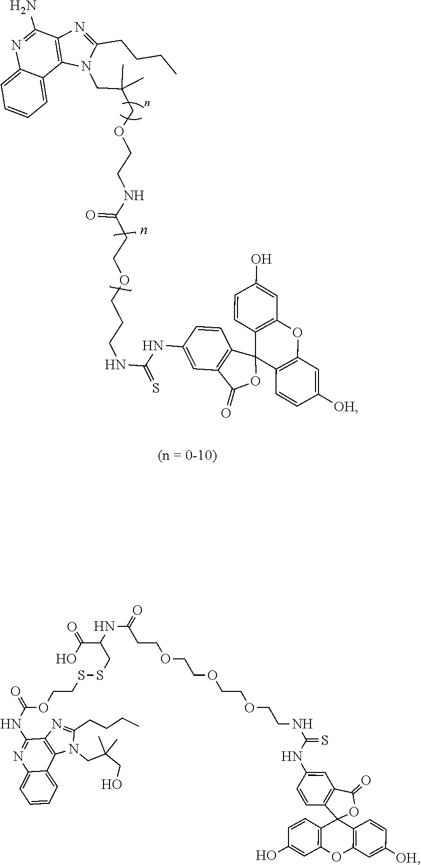 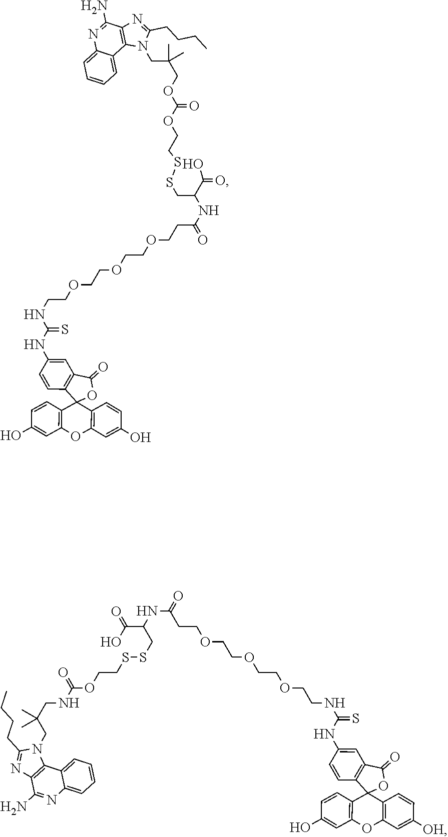 [SMILES: CCCCc1nc2c(N)nc3ccccc3c2n1CC(C)(C)COC(=O)OCCSSCC(NC(=O)CCOCCOCCOCCNC(=S)Nc1ccc2c(c1)C(=O)OC21c2ccc(O)cc2Oc2cc(O)ccc21)C(=O)O ; CCCCc1nc2c(N)nc3ccccc3c2n1CC(C)(C)CNC(=O)OCCSSCC(NC(=O)CCOCCOCCOCCNC(=S)Nc1ccc2c(c1)C(=O)OC21c2ccc(O)cc2Oc2cc(O)ccc21)C(=O)O] 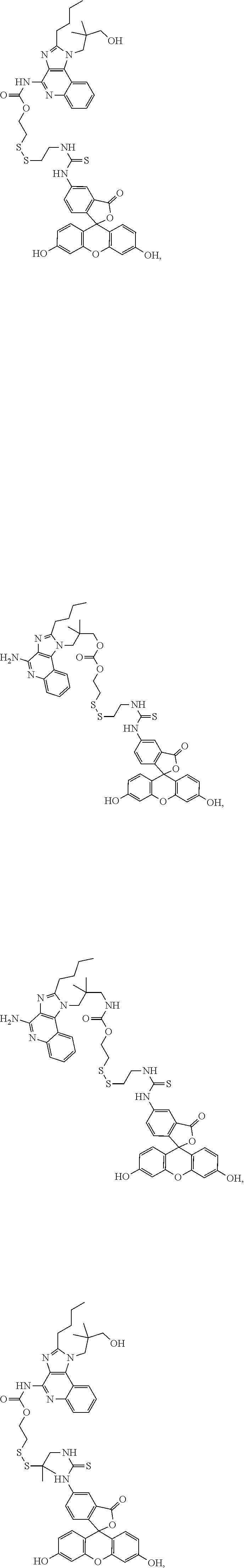 [SMILES: CCCCc1nc2c(NC(=O)OCCSSCCNC(=S)Nc3ccc4c(c3)C(=O)OC43c4ccc(O)cc4Oc4cc(O)ccc43)nc3ccccc3c2n1CC(C)(C)CO ; CCCCc1nc2c(N)nc3ccccc3c2n1CC(C)(C)COC(=O)OCCSSCCNC(=S)Nc1ccc2c(c1)C(=O)OC21c2ccc(O)cc2Oc2cc(O)ccc21 ; CCCCc1nc2c(N)nc3ccccc3c2n1CC(C)(C)CNC(=O)OCCSSCCNC(=S)Nc1ccc2c(c1)C(=O)OC21c2ccc(O)cc2Oc2cc(O)ccc21 ; CCCCc1nc2c(NC(=O)OCCSSC(C)(C)CNC(=S)Nc3ccc4c(c3)C(=O)OC43c4ccc(O)cc4Oc4cc(O)ccc43)nc3ccccc3c2n1CC(C)(C)CO] 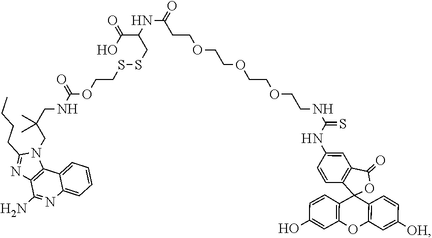 [SMILES: CCCCc1nc2c(N)nc3ccccc3c2n1CC(C)(C)CNC(=O)OCCSSCC(NC(=O)CCOCCOCCOCCNC(=S)Nc1ccc2c(c1)C(=O)OC21c2ccc(O)cc2Oc2cc(O)ccc21)C(=O)O] 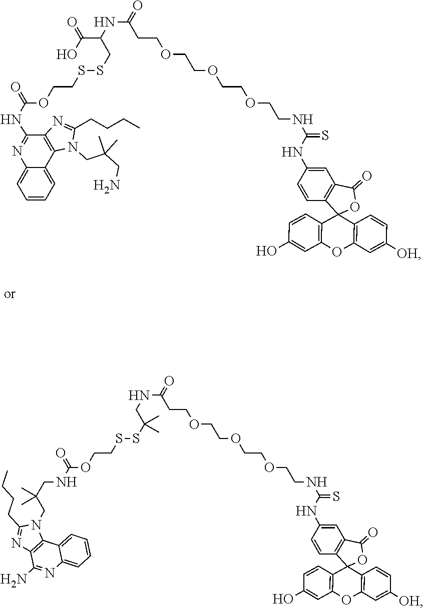 [SMILES: CCCCc1nc2c(NC(=O)OCCSSCC(NC(=O)CCOCCOCCOCCNC(=S)Nc3ccc4c(c3)C(=O)OC43c4ccc(O)cc4Oc4cc(O)ccc43)C(=O)O)nc3ccccc3c2n1CC(C)(C)CN ; CCCCc1nc2c(N)nc3ccccc3c2n1CC(C)(C)CNC(=O)OCCSSC(C)(C)CNC(=O)CCOCCOCCOCCNC(=S)Nc1ccc2c(c1)C(=O)OC21c2ccc(O)cc2Oc2cc(O)ccc21]
or is a pharmaceutically acceptable salt of any of the foregoing structures.

9. The method of claim 1, wherein the CAR T-cell-rejuvenating conjugate has the structure: 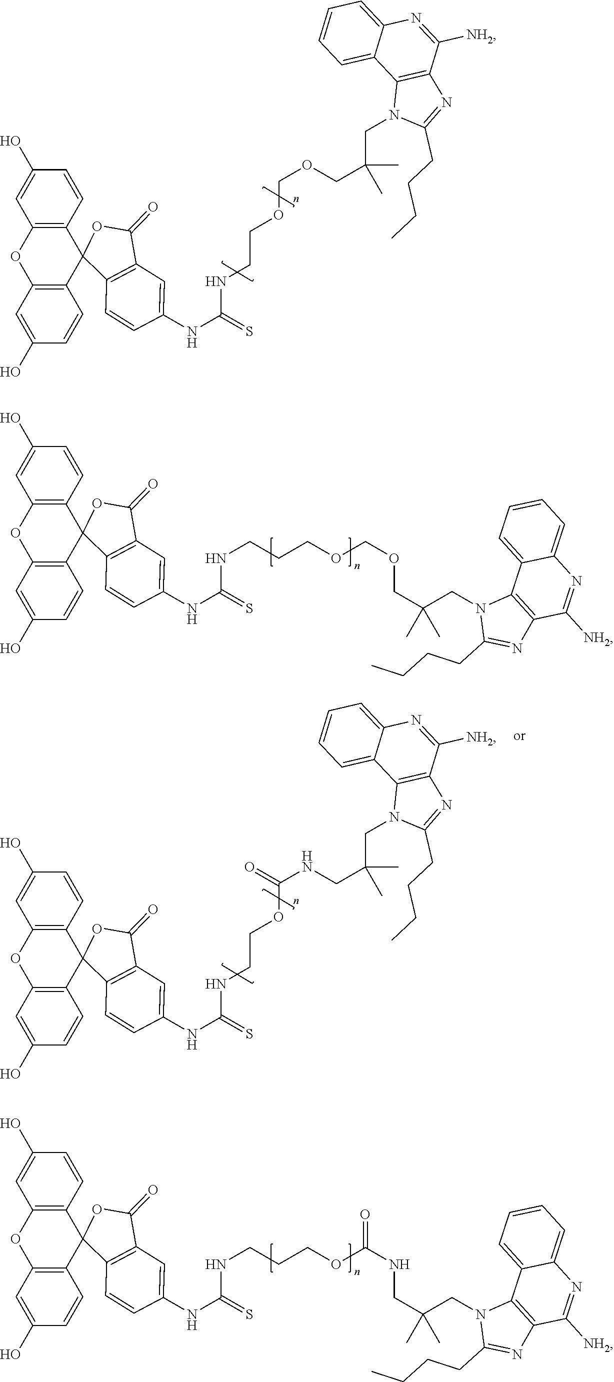
wherein n=0-200, or a pharmaceutically acceptable salt of any of the foregoing structures.

10-11. (canceled)

12. The method of claim 1, wherein the CAR T-cell-rejuvenating conjugate has a structure selected from: 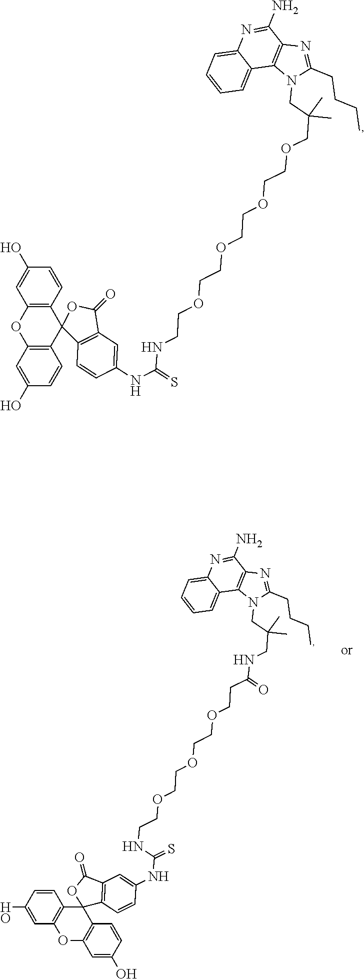 [SMILES: CCCCc1nc2c(N)nc3ccccc3c2n1CC(C)(C)COCCOCCOCCOCCNC(=S)Nc1ccc2c(c1)C(=O)OC21c2ccc(O)cc2Oc2cc(O)ccc21 ; CCCCc1nc2c(N)nc3ccccc3c2n1CC(C)(C)CNC(=O)CCOCCOCCOCCNC(=S)Nc1ccc2c(c1)C(=O)OC21c2ccc(O)cc2Oc2cc(O)ccc21] 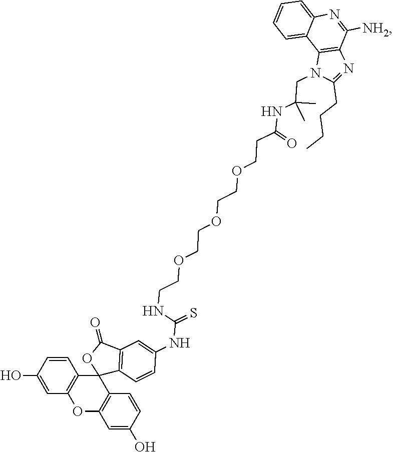 [SMILES: CCCCc1nc2c(N)nc3ccccc3c2n1CC(C)(C)NC(=O)CCOCCOCCOCCNC(=S)Nc1ccc2c(c1)C(=O)OC21c2ccc(O)cc2Oc2cc(O)ccc21] 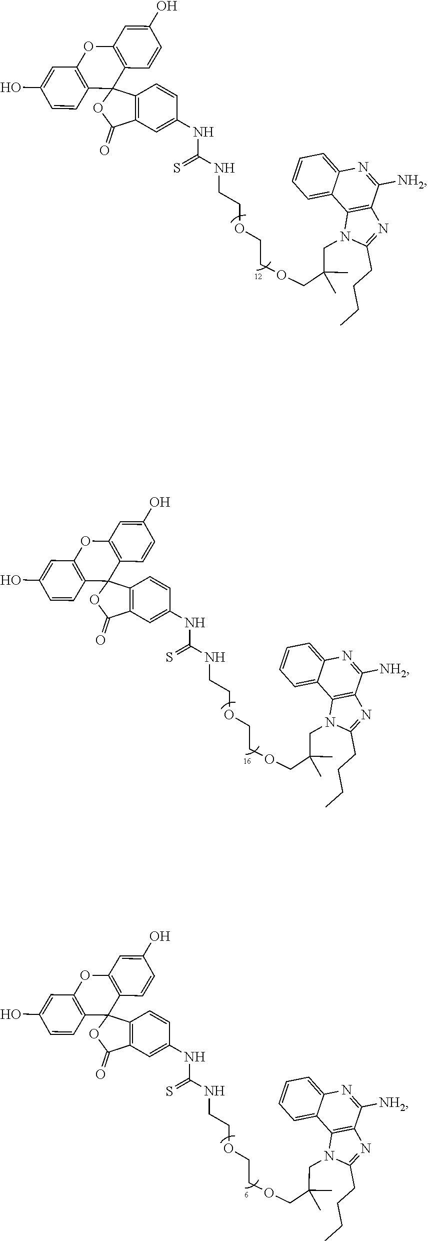 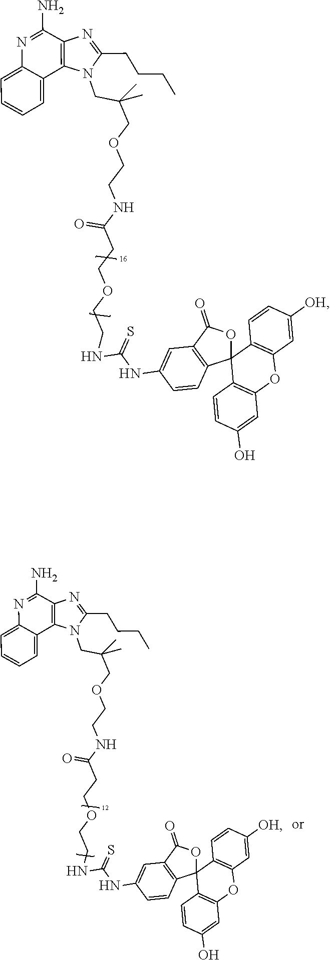 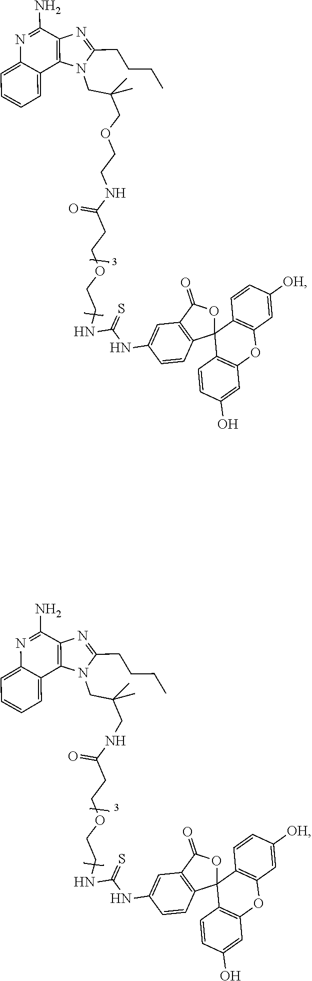 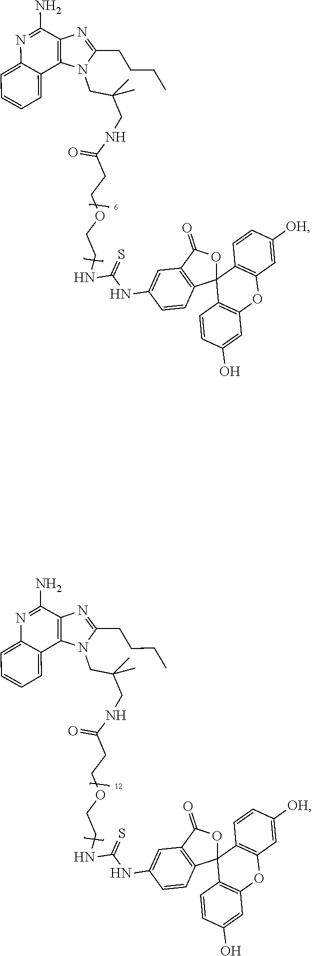 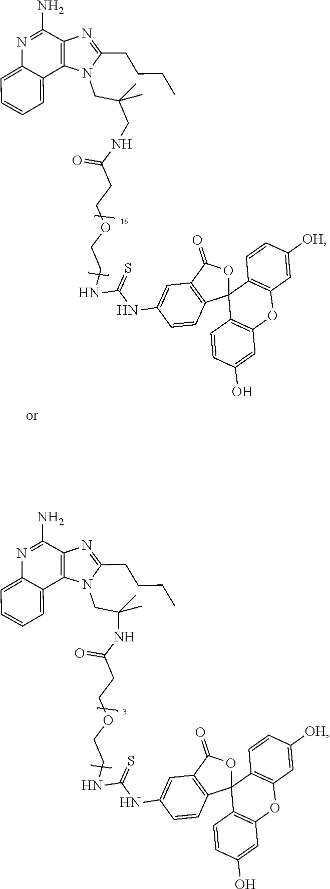 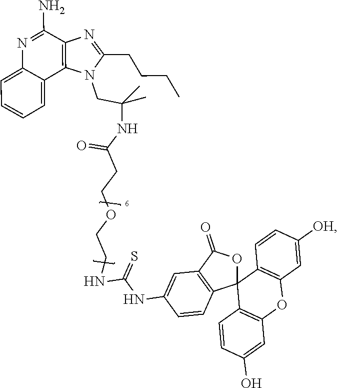
or is a pharmaceutically acceptable salt of any of the foregoing structures.

13-14. (canceled)

15. The method of claim 1, wherein the ligand that binds a cancer cell with specificity is selected from the group consisting of a folate, a 5-methyltetrahydrofolate, a 2-[3-(1,3-dicarboxypropyl)ureido]pentanedioic acid (DUPA) ligand, a neurokinin 1 receptor (NK-1R) ligand, a carbonic anhydrase IX (CAIX) ligand, a ligand of gamma glutamyl transpeptidase, a ligand of luteinizing hormone releasing hormone (LHRHR), a ligand of CD73, a ligand of heat shock protein (HSP), a ligand of glucose transporter 1 (glut-1), a ligand of fibroblast activation protein, a ligand of a natural killer group 2D receptor (NKG2D) ligand, and a cholecystokinin B receptor (CCKBR or CCK2) ligand.

16. The method of claim 1, wherein the first targeting moiety and the second targeting moiety are independently selected from the group consisting of 2,4-dinitrophenyl (DNP), L-rhamnose, tacrolimus (FK506), 2,4,6-trinitrophenol (TNP), biotin, rapamycin, digoxigenin, folate, 5-methyl tetrahydrofolate, fluorescein, fluorescein isothiocyanate (FITC), NHS-fluorescein, pentafluorophenyl ester, tetrafluorophenyl ester, knottin, centyrin, and DARPin.

17. The method of claim 1, wherein:
the first targeting moiety and the second targeting moiety are independently selected from the group consisting of DNP, FK506, TNP, biotin, rapamycin, digoxigenin, folate, 5-methyl tetrahydrofolate, fluorescein, FITC, NHS-fluorescein, pentafluorophenyl ester, tetrafluorophenyl ester, knottin, centyrin, and DARPin; and
the ligand that binds a cancer cell with specificity is selected from the group consisting of a folate, a DUPA ligand, a NK-1R ligand, a CAIX ligand, a ligand of gamma glutamyl transpeptidase, a ligand of LHRHR, a ligand of CD73, a ligand of HSP, a ligand of glut-1, a ligand of fibroblast activation protein, a ligand of a NKG2D ligand, and a CCKBR or CCK2 ligand.

18. The method of claim 1, wherein the first linker and the second linker are independently releasable or non-releasable; and/or the first linker, the second linker, or both the first linker and the second linker comprise PEG.

19. The method of claim 1, wherein the first linker and the second linker independently comprise C₁-C₂₀ alkyl, alkylene, heteroalkylene, —O-alkynylene, alkenylene, acyl, aryl, heteroaryl, amide, oxime, ether, ester, triazole, —S—S, —CO—O—(CH₂)ₙ—S—S— (where n=2-6), —O—CO—O—(CH₂)ₙ—S—S— (where n=2-6), —S—CO—O—(CH₂)ₙ—S—S— (where n=2-6), —NH—CO—O—(CH₂)ₙ—S—S— (where n=2-6), carboxylate, carbonate, carbamate, urea, thiourea, polyethylene glycol (PEG), polyproline, oligo-(4-piperidine) carboxylic acid, oligo piperidine, amino acid, peptide, saccharo-peptide, sugar, peptidoglycan, a polyvinylpyrrolidone, pluronic F-127, or any combination of two or more of the foregoing.

20. (canceled)

21. A chimeric antigen receptor (CAR) T-cell-rejuvenating conjugate comprising an agonist of toll-like receptor 7, toll-like receptor 8, or toll-like receptor 7 and toll-like receptor 8 conjugated via a linker with a targeting moiety that binds a CAR of a CAR T-cell with specificity, wherein the CAR T-cell-rejuvenating conjugate has the structure: 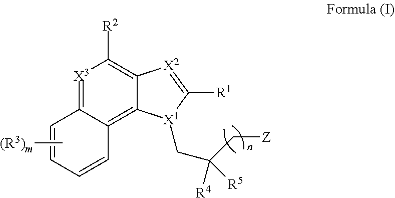
or is a pharmaceutically acceptable salt thereof, wherein:
R¹ is an acyclic or cyclic alkyl, which is optionally substituted with one or more substituents independently selected from an alkyl, halo, heteroalkyl, alkoxy, and cycloalkyl;
R² is —NR^{2x}R^{2y}, H, —OR^{z}, —SO₂N(R^{z})₂, or N₃, wherein:
R^{2x} and R^{2y} are each independently hydrogen (H), —N(R^{z})₂, —CON(R^{z})₂, —C(R^{z})₂—N(R^{z})₂, —CS—N(R^{z})₂, or an alkyl, which is optionally substituted with one or more substituents independently selected from oxo, halo, alkyl, heteroalkyl, alkoxy, and cycloalkyl;
each R^{z} is independently H or alkyl that is optionally substituted; or
R^{2x} and R^{2y} are taken together to form a 3-10 membered mono- or bicyclic heterocycloalkyl, which is optionally substituted;
each R³ is independently H, halo, —N₃, —CN, —NO₂, alkyl, heteroalkyl, cycloalkyl, heterocycloalkyl, alkoxy, aryl, heteroaryl, heterocycloalkyl, amino, hydroxy, carbonyl, or thiol, wherein the alkyl, heteroalkyl, cycloalkyl, heterocycloalkyl, or alkoxy that is optionally substituted;
R⁴ and R⁵ are each independently alkyl, alkoxy, halo, or cycloalkyl, wherein the alkyl, alkoxy, or cycloalkyl that is optionally substituted;
X¹, X², and X³ are independently N or CR^{q}, wherein each R^{q} is independently H, halo, or optionally substituted alkyl;
Z is G-L-, wherein L is a linker and G is a targeting moiety;
n in Formula (I) is 1-6; and
m in Formula (I) is 0-4; or
wherein the CAR T-cell-rejuvenating conjugate has the structure: 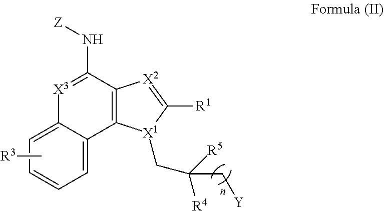
or is a pharmaceutically acceptable salt thereof wherein:
R¹, R³, R⁴, and R⁵ are each independently H, alkyl, alkoxyl, alkenyl, alkynyl, alicyclic, aryl, biaryl, halo, heteroaryl, —COR^{2x} 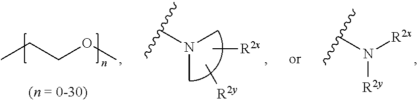
wherein each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl;
Z is G-L-, wherein L is a linker and G is a targeting moiety;
X¹, X², and X³ are independently CR^{q} or N, wherein each R^{q} is independently H, halo, or optionally substituted alkyl;
Y is H, —OR^{z}, —NR^{2x}R^{2y}, —SR^{z}, —SOR^{z}, —SO₃R^{z}, —N₃, —COR^{z}, —COOR^{z}, —CON(R^{z})₂, —COSR^{z}, —SO₂N(R^{z})₂, or —CON(R^{z})₂, wherein:
R^{2x} and R^{2y} are each independently hydrogen, —N(R^{z})₂, —CON(R^{z})₂, —C(R^{z})₂—N(R^{z})₂, —CS—N(R^{z})₂, or optionally substituted alkyl;
each R^{z} is independently H or optionally substituted alkyl; or
R^{2x} and R^{2y} are taken together to form an optionally substituted heterocycloalkyl; and
n in Formula (II) is 0-30; or
wherein the CAR T-cell-rejuvenating conjugate has the structure: 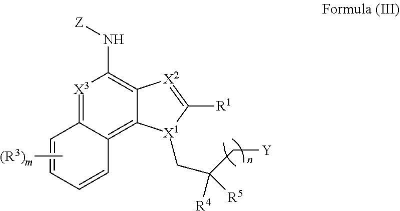
or is a pharmaceutically acceptable salt thereof, wherein:
R¹ is an acyclic or cyclic alkyl, which is optionally substituted with one or more substituents independently selected from halo, alkyl, heteroalkyl, alkoxy, and cycloalkyl;
Y is H, —OR^{z}, —NR^{2x}R^{2y}, —SR^{z}, —SOR^{z}, —SO₃R^{z}, —N₃, —COR^{z}, —COOR^{z}, —CONR^{z}₂, —COSR^{z}, —SO₂N(R^{z})₂, or —CON(R^{z})₂, wherein:
each R^{z} is independently H or optionally substituted alkyl;
R^{2x} and R^{2y} are each independently H, —N(R^{z})₂, —CON(R^{z})₂, —C(R^{z})₂—N(R^{z})₂, —CS—N(R^{z})₂, or alkyl that is optionally substituted with one or more substituents independently selected from oxo, halo, alkyl, heteroalkyl, alkoxy, and cycloalkyl; or
R^{2x} and R^{2y} are taken together to form a 3-10 membered mono- or bicyclic heterocycloalkyl, which is optionally substituted;
each R³ is independently halo, —N₃, —CN, —NO₂, alkyl, heteroalkyl, cycloalkyl, heterocycloalkyl, alkoxy, aryl, heteroaryl, heterocycloalkyl, amino, hydroxy, carbonyl, or thiol, wherein the alkyl, alkoxy, heteroalkyl, cycloalkyl, or heterocycloalkyl is optionally substituted;
R⁴ and R⁵ are each independently alkyl, alkoxy, halogen, or cycloalkyl, wherein the alkyl, alkoxy, or cycloalkyl is optionally substituted;
X¹, X², and X³ are independently CR^{q} or N, wherein each R^{q} is independently hydrogen, halogen, or optionally substituted alkyl;
Z is L-G, wherein L is a linker and G is a targeting moiety;
n in Formula (III) is 0-30; and
m in Formula (III) is 0-4; and
wherein G in Formula (I), Formula (II) or Formula (III) is a group or comprises a group with the structure: 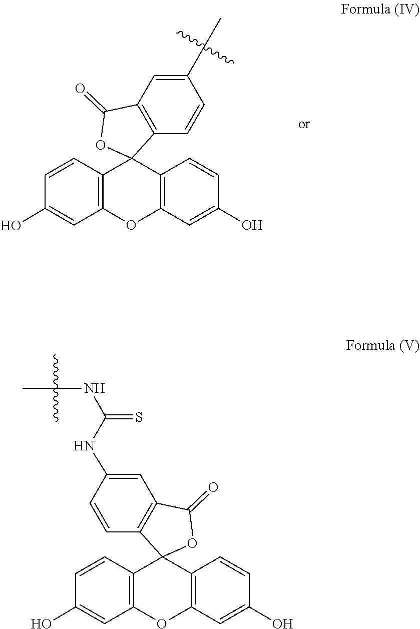 [SMILES: *c1ccc2c(c1)C(=O)OC21c2ccc(O)cc2Oc2cc(O)ccc21 ; *NC(=S)Nc1ccc2c(c1)C(=O)OC21c2ccc(O)cc2Oc2cc(O)ccc21]
or is a pharmaceutically acceptable salt thereof.

22. The CAR T-cell-rejuvenating conjugate of claim 21, wherein each of X¹, X², and X³ of Formula (I), Formula (II), and Formula (III) is N.

23. The CAR T-cell-rejuvenating conjugate of claim 21, which has the structure: 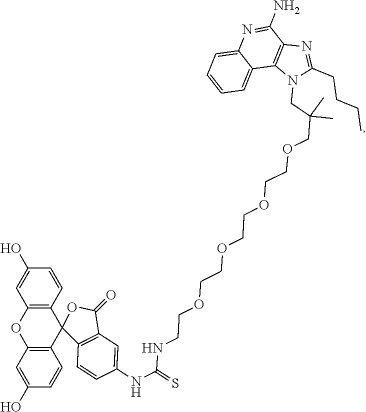 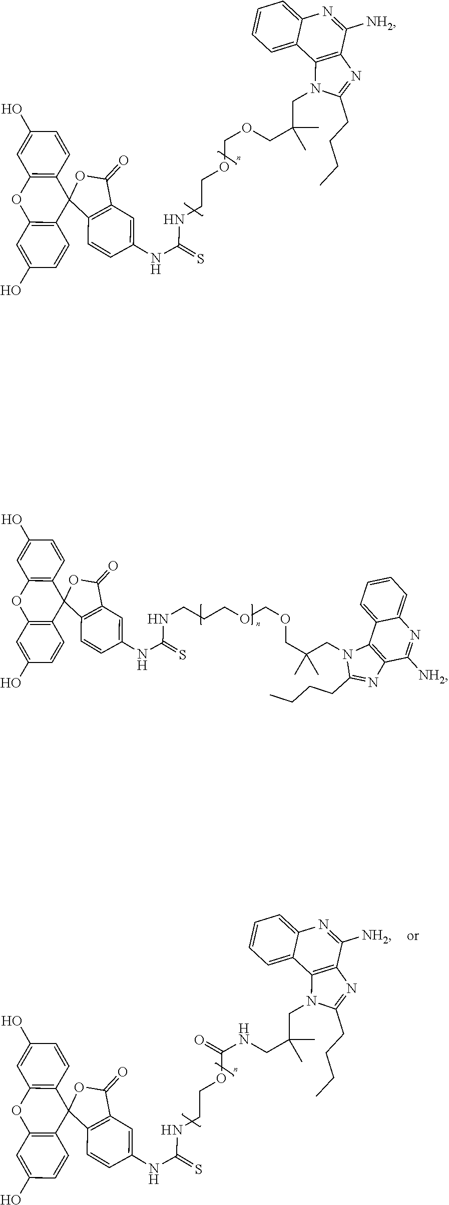 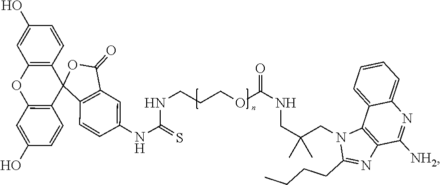
wherein n=200, or a pharmaceutically acceptable salt of any of the foregoing structures.

24. (canceled)

25. The CAR T-cell-rejuvenating conjugate of claim 21, which has a structure selected from: 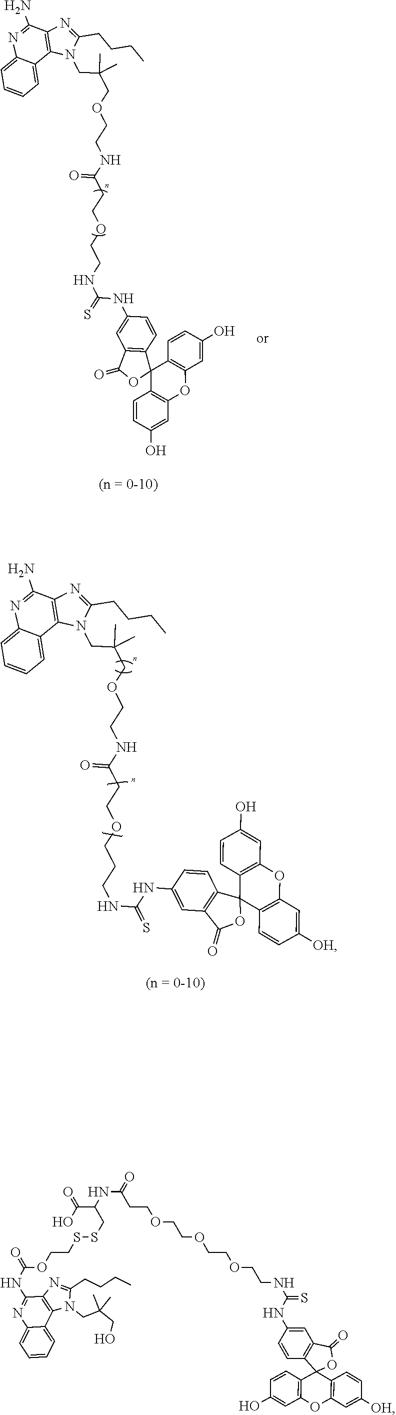 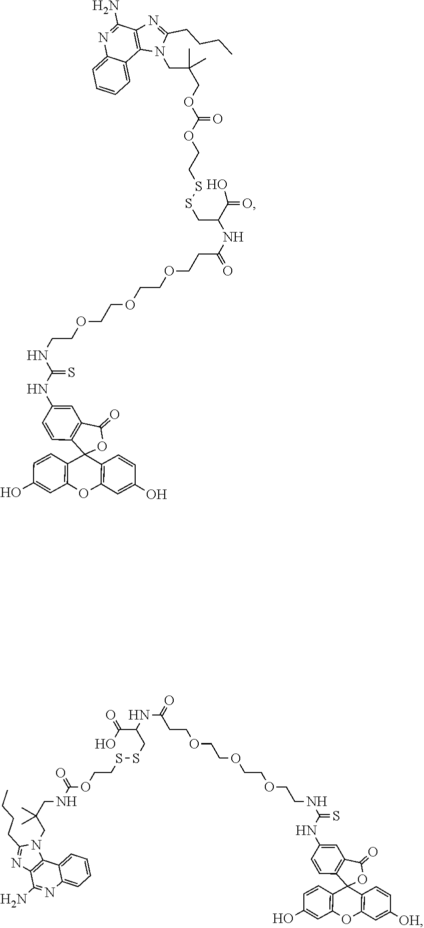 [SMILES: CCCCc1nc2c(N)nc3ccccc3c2n1CC(C)(C)COC(=O)OCCSSCC(NC(=O)CCOCCOCCOCCNC(=S)Nc1ccc2c(c1)C(=O)OC21c2ccc(O)cc2Oc2cc(O)ccc21)C(=O)O ; CCCCc1nc2c(N)nc3ccccc3c2n1CC(C)(C)CNC(=O)OCCSSCC(NC(=O)CCOCCOCCOCCNC(=S)Nc1ccc2c(c1)C(=O)OC21c2ccc(O)cc2Oc2cc(O)ccc21)C(=O)O] 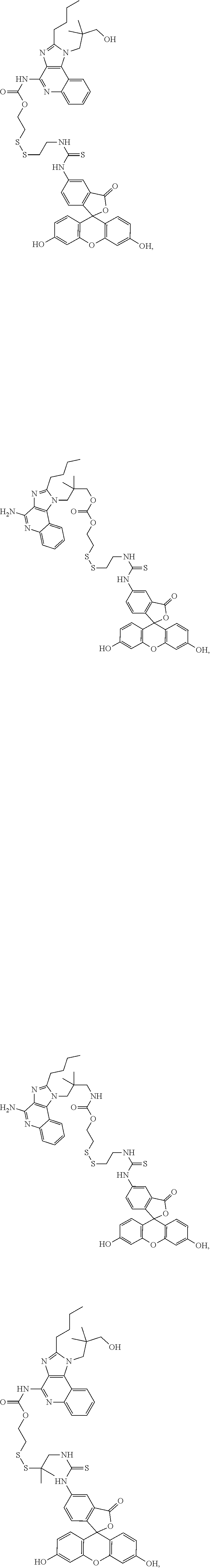 [SMILES: CCCCc1nc2c(NC(=O)OCCSSCCNC(=S)Nc3ccc4c(c3)C(=O)OC43c4ccc(O)cc4Oc4cc(O)ccc43)nc3ccccc3c2n1CC(C)(C)CO ; CCCCc1nc2c(N)nc3ccccc3c2n1CC(C)(C)COC(=O)OCCSSCCNC(=S)Nc1ccc2c(c1)C(=O)OC21c2ccc(O)cc2Oc2cc(O)ccc21 ; CCCCc1nc2c(N)nc3ccccc3c2n1CC(C)(C)CNC(=O)OCCSSCCNC(=S)Nc1ccc2c(c1)C(=O)OC21c2ccc(O)cc2Oc2cc(O)ccc21 ; CCCCc1nc2c(NC(=O)OCCSSC(C)(C)CNC(=S)Nc3ccc4c(c3)C(=O)OC43c4ccc(O)cc4Oc4cc(O)ccc43)nc3ccccc3c2n1CC(C)(C)CO] 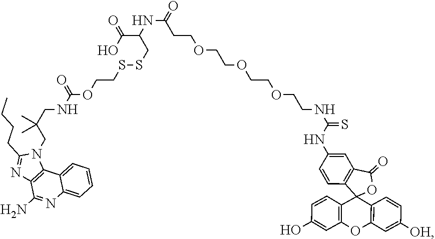 [SMILES: CCCCc1nc2c(N)nc3ccccc3c2n1CC(C)(C)CNC(=O)OCCSSCC(NC(=O)CCOCCOCCOCCNC(=S)Nc1ccc2c(c1)C(=O)OC21c2ccc(O)cc2Oc2cc(O)ccc21)C(=O)O] 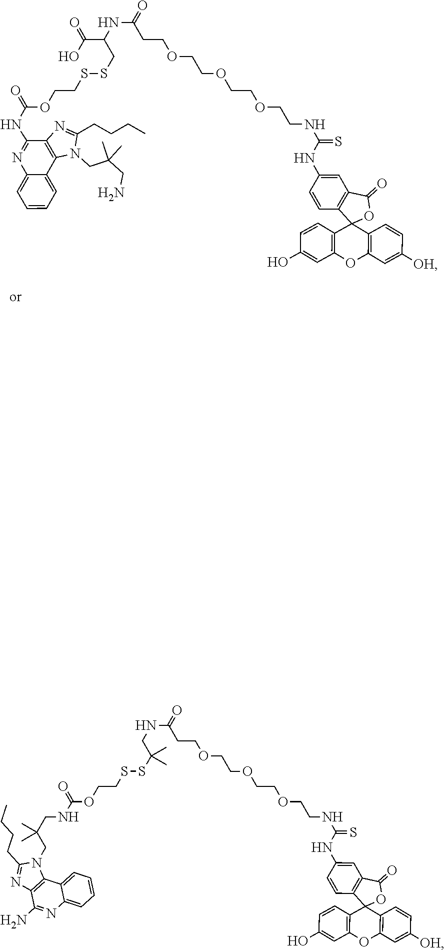 [SMILES: CCCCc1nc2c(NC(=O)OCCSSCC(NC(=O)CCOCCOCCOCCNC(=S)Nc3ccc4c(c3)C(=O)OC43c4ccc(O)cc4Oc4cc(O)ccc43)C(=O)O)nc3ccccc3c2n1CC(C)(C)CN ; CCCCc1nc2c(N)nc3ccccc3c2n1CC(C)(C)CNC(=O)OCCSSC(C)(C)CNC(=O)CCOCCOCCOCCNC(=S)Nc1ccc2c(c1)C(=O)OC21c2ccc(O)cc2Oc2cc(O)ccc21] 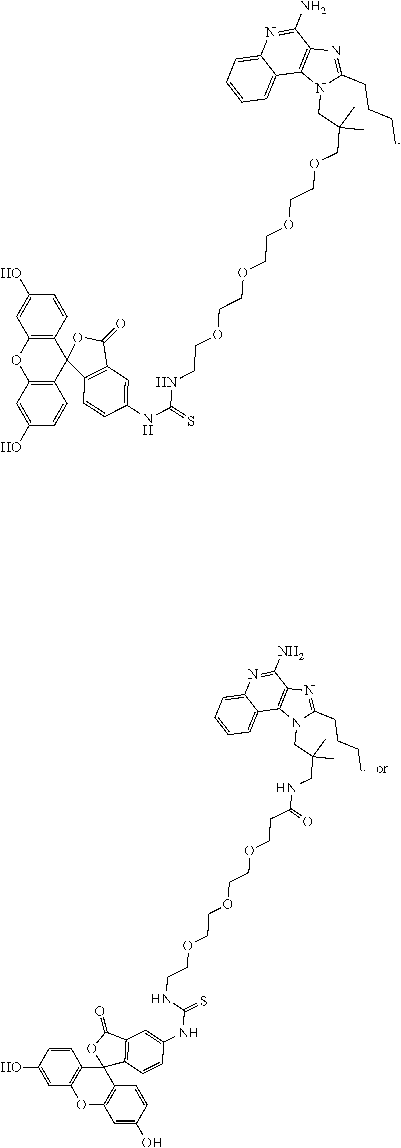 [SMILES: CCCCc1nc2c(N)nc3ccccc3c2n1CC(C)(C)COCCOCCOCCOCCNC(=S)Nc1ccc2c(c1)C(=O)OC21c2ccc(O)cc2Oc2cc(O)ccc21 ; CCCCc1nc2c(N)nc3ccccc3c2n1CC(C)(C)CNC(=O)CCOCCOCCOCCNC(=S)Nc1ccc2c(c1)C(=O)OC21c2ccc(O)cc2Oc2cc(O)ccc21] 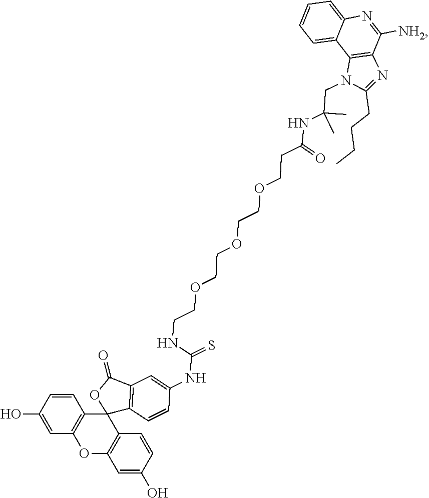 [SMILES: CCCCc1nc2c(N)nc3ccccc3c2n1CC(C)(C)NC(=O)CCOCCOCCOCCNC(=S)Nc1ccc2c(c1)C(=O)OC21c2ccc(O)cc2Oc2cc(O)ccc21]
or a pharmaceutically acceptable salt of any of the foregoing structures.

26-27. (canceled)

28. The CAR T-cell-rejuvenating conjugate of claim 21, which has a structure selected from: 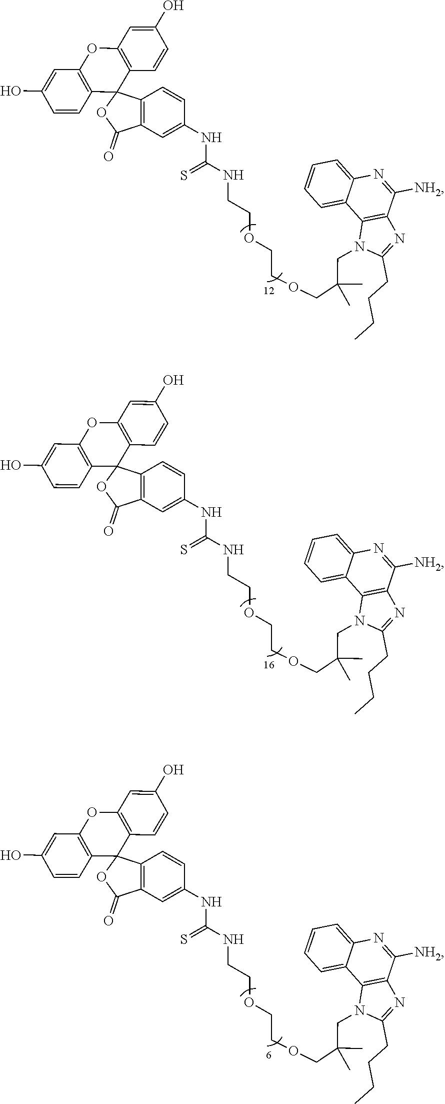 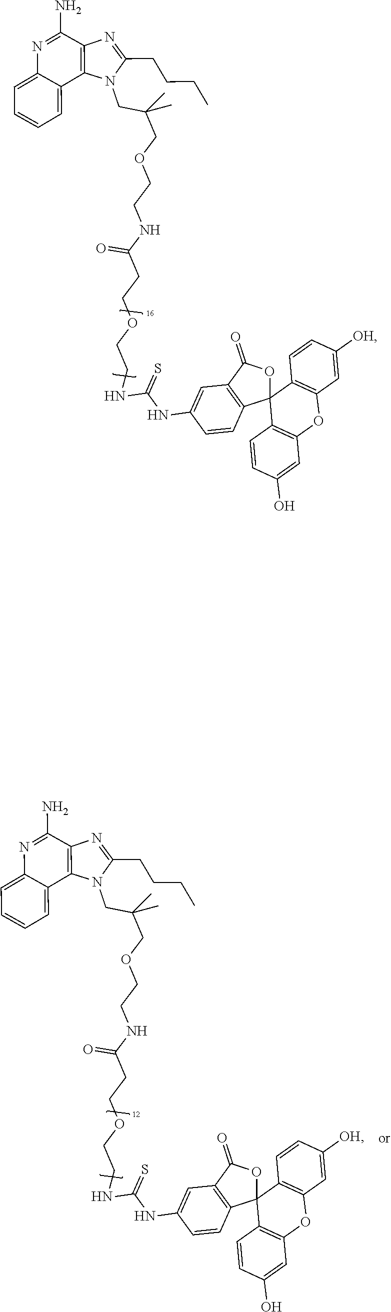 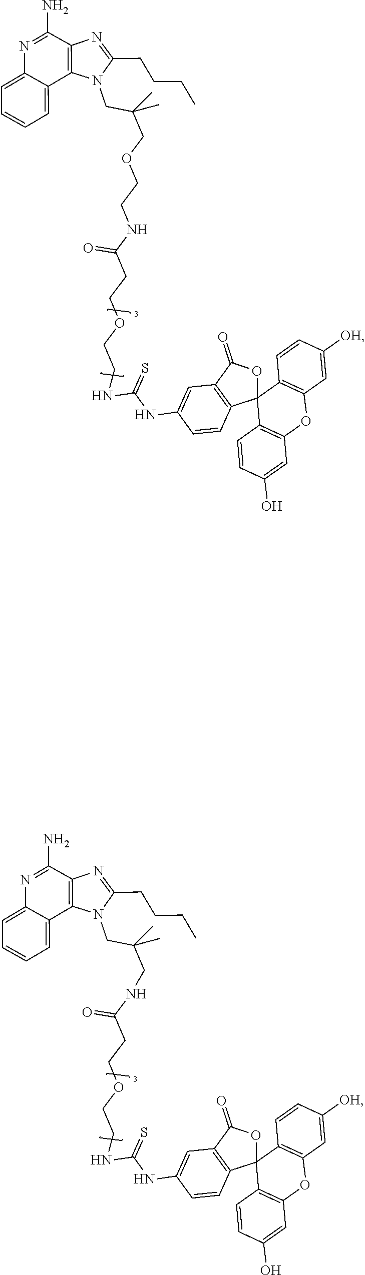 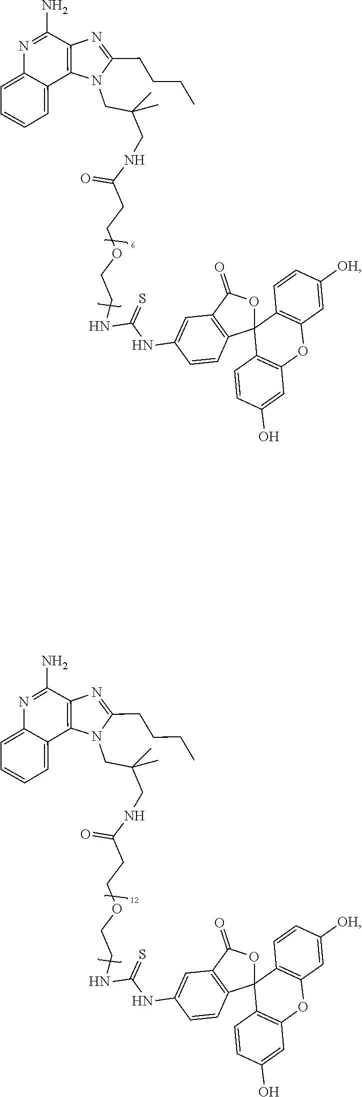 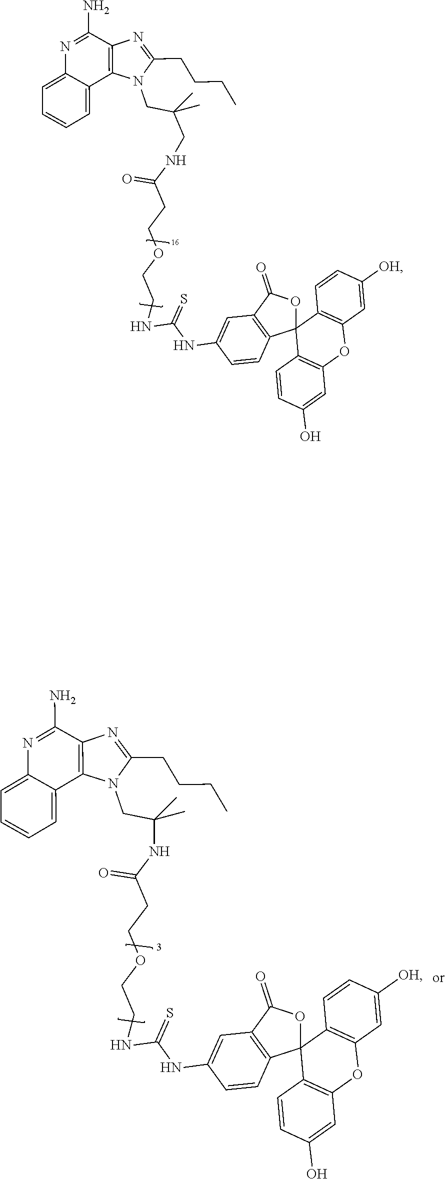 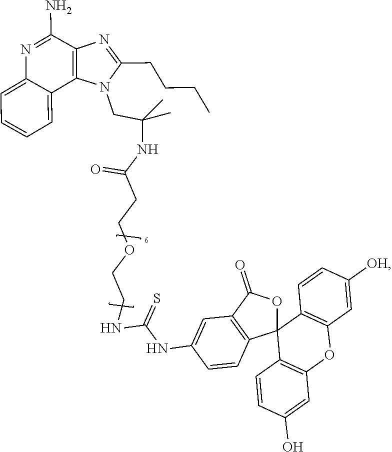
or is a pharmaceutically acceptable salt of any of the foregoing structures.

29. (canceled)

30. The CAR T-cell-rejuvenating conjugate of claim 21, wherein the linker is releasable or non-releasable.

31. The CAR T-cell-rejuvenating conjugate of claim 21, wherein the linker comprises C₁-C₂₀alkyl, alkylene, heteroalkylene, —O-alkynylene, alkenylene, acyl, aryl, heteroaryl, amide, oxime, ether, ester, triazole, carboxylate, carbonate, carbamate, urea, thiourea, —S—S—, —CO—O—(CH₂)ₙ—S—S— (where n=2-6), —O—CO—O—(CH₂)ₙ—S—S— (where n=2-6), —S—CO—O—(CH₂)ₙ—S—S— (where n=2-6), —NH—CO—O—(CH₂)ₙ—S—S— (where n=2-6), PEG, polyproline, oligo-(4-piperidine) carboxylic acid, oligo piperidine, amino acid (e.g., hydrophilic amino acid), peptide, saccharo-peptide, sugar, peptidoglycan, a polyvinylpyrrolidone, pluronic F-127, or any combination of two or more of the foregoing.

32. The CAR T-cell-rejuvenating conjugate of claim 21, wherein the linker comprises PEG.

33. A pharmaceutical composition comprising the CAR T-cell-rejuvenating conjugate and a pharmaceutically acceptable carrier, wherein the chimeric antigen receptor (CAR) T-cell rejuvenating conjugate comprises an agonist of toll-like receptor 7, toll-like receptor 8, or toll-like receptor 7 and toll-like receptor 8 conjugated via a linker with a targeting moiety that binds a CAR of a CAR T-cell with specificity, wherein the CAR T-cell-rejuvenating conjugate has the structure: 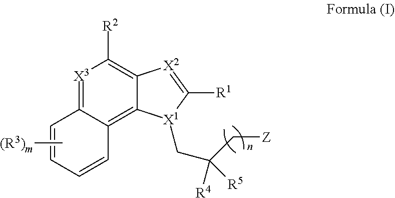
or is a pharmaceutically acceptable salt thereof, wherein:
R¹ is an acyclic or cyclic alkyl, which is optionally substituted with one or more substituents independently selected from an alkyl, halo, heteroalkyl, alkoxy, and cycloalkyl;
R² is —NR^{2x}R^{2y}, H, —OR^{Z}, —SO₂N(R^{Z})₂, or N₃ wherein:
R^{2x} and R^{2y} are each independently hydrogen (H), —N(R^{z})₂, —CON(R^{z})₂, —C(R^{z})₂—N(R^{z})₂, —CS—N(R^{z})₂, or an alkyl, which is optionally substituted with one or more substituents independently selected from oxo, halo, alkyl, heteroalkyl, alkoxy, and cycloalkyl;
each R^{z} is independently H or alkyl that is optionally substituted; or
R^{2x} and R^{2y} are taken together to form a 3-10 membered mono- or bicyclic heterocycloalkyl, which is optionally substituted:
each R³ is independently H, halo, —N₃, —CN, —NO₂, alkyl, heteroalkyl, cycloalkyl, heterocycloalkyl, alkoxy, aryl, heteroaryl, heterocycloalkyl, amino, hydroxy, carbonyl, or thiol, wherein the alkyl, heteroalkyl, cycloalkyl, heterocycloalkyl, or alkoxy that is optionally substituted;
R⁴ and R⁵ are each independently alkyl, alkoxy, halo, or cycloalkyl, wherein the alkyl, alkoxy, or cycloalkyl that is optionally substituted;
X¹, X², and X³ are independently N or CR^{q}, wherein each R^{q} is independently H, halo, or optionally substituted alkyl;
Z is G-L-, wherein L is a linker and G is a targeting moiety;
n in Formula (I) is 1-6; and
m in Formula (I) is 0-4: or
wherein the CAR T-cell-rejuvenating conjugate has the structure: 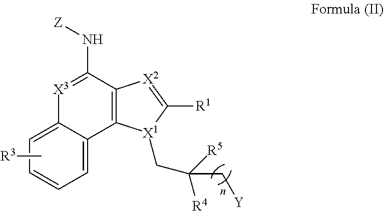
or is a pharmaceutically acceptable salt thereof wherein:
R¹, R³, R⁴, and R⁵ are each independently H, alkyl, alkoxyl, alkenyl, alkynyl, alicyclic, aryl, biaryl, halo, heteroaryl, —COR^{2x}, 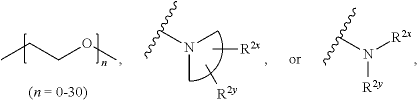
wherein each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl;
Z is G-L-, wherein L is a linker and G is a targeting moiety;
X¹, X², and X³ are independently CR^{q} or N, wherein each R^{q} is independently H, halo, or optionally substituted alkyl;
Y is H, —OR^{z}, —NR^{2x}R^{2y}, —SR^{z}, —SOR^{z}, —SO₃R^{z}, —N₃, —COR^{z}, —COOR^{z}, —CON(R^{z})₂, —COSR^{z}, —SO₂N(R^{z})₂, or —CON(R^{z})₂, wherein:
R^{2x} and R^{2y} are each independently hydrogen, —N(R^{z})₂, —CON(R^{z})₂, —C(R^{z})₂—N(R^{z})₂, —CS—N(R^{z})₂, or optionally substituted alkyl;
each R^{z} is independently H or optionally substituted alkyl; or
R^{2x} and R^{2y} are taken together to form an optionally substituted heterocycloalkyl; and
n in Formula (II) is 0-30: or
wherein the CAR T-cell-rejuvenating conjugate has the structure: 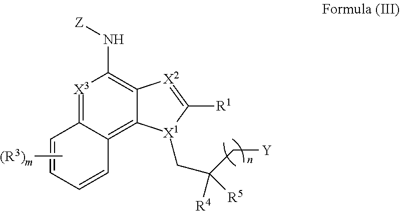
or is a pharmaceutically acceptable salt thereof wherein:
R¹ is an acyclic or cyclic alkyl, which is optionally substituted with one or more substituents independently selected from halo, alkyl, heteroalkyl, alkoxy, and cycloalkyl;
Y is H, —OR^{z}, —NR^{2x}R^{2y}, —SR^{z}, —SOR^{z}, —SO₃R^{z}, —N₃, —COR^{z}, —COOR^{z}, —CONR^{z}₂, —COSR^{z}, —SO₂N(R^{z})₂, or —CON(R^{z})₂, wherein:
each R^{Z} is independently H or optionally substituted alkyl;
R^{2x} and R^{2y} are each independently H, —N(R^{z})₂, —CON(R^{z})₂, —C(R^{z})₂—N(R^{z})₂, —CS—N(R^{z})₂, or alkyl that is optionally substituted with one or more substituents independently selected from oxo, halo, alkyl, heteroalkyl, alkoxy, and cycloalkyl; or
R^{2x} and R^{2y} are taken together to form a 3-10 membered mono- or bicyclic heterocycloalkyl, which is optionally substituted:
each R³ is independently halo, —N₃, —CN, —NO₂, alkyl, heteroalkyl, cycloalkyl, heterocycloalkyl, alkoxy, aryl, heteroaryl, heterocycloalkyl, amino, hydroxy, carbonyl, or thiol, wherein the alkyl, alkoxy, heteroalkyl, cycloalkyl, or heterocycloalkyl is optionally substituted;
R⁴ and R⁵ are each independently alkyl, alkoxy, halogen, or cycloalkyl, wherein the alkyl, alkoxy, or cycloalkyl is optionally substituted;
X¹, X², and X³ are independently CR^{q} or N, wherein each R_{q} is independently hydrogen, halogen, or optionally substituted alkyl;
Z is L-G, wherein L is a linker and G is a targeting moiety;
n in Formula (III) is 0-30; and
m in Formula (III) is 0-4; and
wherein G in Formula (I), Formula (II) or Formula (III) is a group or comprises a group with the structure: 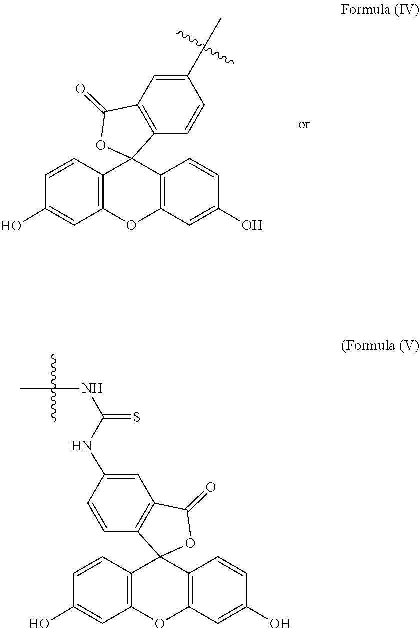 [SMILES: *c1ccc2c(c1)C(=O)OC21c2ccc(O)cc2Oc2cc(O)ccc21 ; *NC(=S)Nc1ccc2c(c1)C(=O)OC21c2ccc(O)cc2Oc2cc(O)ccc21]
or is a pharmaceutically acceptable salt thereof.
